SEQUENCE LISTING (126 sequences)

SEQ ID NO: 1 - protein, 8 aa, Artificial Sequence
  homo sapiens
GYSFTSYW

SEQ ID NO: 2 - protein, 8 aa, Artificial Sequence
  homo sapiens
  misc_feature (5)..(5): Xaa can be Asp, Glu, or Gln
  misc_feature (5)..(6): when Xaa at position 5 is Asp, Xaa at position 6 is different than Ser
  misc_feature (6)..(6): Xaa can be Ser, Ile, or Thr
IYPGXXDT

SEQ ID NO: 3 - protein, 19 aa, Artificial Sequence
  homo sapiens
ARHDVGYCTDRTCAKWPEY

SEQ ID NO: 4 - protein, 8 aa, Artificial Sequence
  homo sapiens and Escherichia virus M13
SSNIGNNY

SEQ ID NO: 5 - protein, 3 aa, Artificial Sequence
  homo sapiens and Escherichia virus M13
DHT

SEQ ID NO: 6 - protein, 11 aa, Artificial Sequence
  homo sapiens and Escherichia virus M13
ASWDYTLSGWV

SEQ ID NO: 7 - DNA, 1753 bp, Artificial Sequence
  homo sapiens and Escherichia virus M13
aagcttgccgccaccatggaatggtcctgggtgttcctgttcttcctgtccgtgaccaccggcgtgcactctcaggttcagttggttcagtctggcgccgaagtgaagaagcctggcgagtccctgaagatctcctgcaaaggctccggctactccttcacctcctactggatcgcctgggtccgacagatgcctggaaagggcctcgagtacatgggcctgatctaccctggcgactccgataccaagtactcccctagcttccagggccaagtgaccatctccgtggacaagtctgtgtccaccgcctacctgcagtggtcctctctgaagccttctgactccgccgtgtacttctgcgccaggcacgatgtgggctactgcaccgatagaacctgcgccaagtggcccgagtactttcagcattggggccagggcacactggtcaccgtttcttccgcctctacacagtcccctagcgtgttccctctgaccagatgctgcaagaacatcccctccaacgccacctctgtgaccctgggatgtctggccaccggctattttcccgagcctgtgatggtcacctgggacaccggatctctgaacggcaccaccatgacactgcctgccaccacactgaccctgtctggacactacgctaccatcagcctgctgacagtgtctggcgcttgggccaagcagatgttcacctgtagagtggctcacaccccttccagcaccgactgggtcgacaacaagaccttctccgtgtgctcccgggacttcacccctcctacagtgaagatcctgcagtcctcttgtgatggcggcggacacttcccacctacaatccagctgctgtgcctggtgtctggctatacccctggcaccatcaacatcacctggctggaagatggacaagtgatggacgtggacctgagcaccgccagcacaacacaagaaggcgagctggcttctacccagtccgagctgacactgtcccagaagcactggctgtccgaccggacctacacctgtcaagtgacctaccagggccacaccttcgaggactccaccaagaagtgcgccgacagcaatcctagaggcgtgtccgcttacctgagccggccttctcctttcgacctgttcatccggaagtctcccaccatcacatgtctggtggtggatctggccccttccaagggcaccgtgaatctgacctggtctagagcctctggcaagcccgtgaaccacagcacccggaaagaggaaaagcagcgcaacggcaccctgaccgtgacctctacactgccagtgggcaccagagattggatcgagggcgagacataccagtgcagagtgacccatcctcatctgcctcgggctctgatgcggtctaccaccaagacctctggacctagagctgcccctgaggtgtacgcctttgctacacctgagtggcctggctccagagacaagagaaccctggcctgcctgatccagaacttcatgcccgaggacatcagcgtgcagtggctgcacaatgaggtgcagctgcctgacgctcggcactctacaacccagcctagaaagaccaaaggcagcggcttcttcgtgttctctcggctggaagtgaccagagccgagtgggagcagaaggacgagttcatctgcagagccgtgcacgaggctgcctctccatctcagacagttcagagggccgtgtctgtgaaccccggcaagtaatgaattc

SEQ ID NO: 8 - DNA, 1753 bp, Artificial Sequence
  homo sapiens and Escherichia virus M13
aagcttgccgccaccatggaatggtcctgggtgttcctgttcttcctgtccgtgaccaccggcgtgcactctcaggttcagttggttcagtctggcgccgaagtgaagaagcctggcgagtccctgaagatctcctgcaaaggctccggctactccttcacctcctactggatcgcctgggtccgacagatgcctggaaagggcctcgagtacatgggcctgatctatcccggcgagtccgataccaagtactcccctagcttccagggccaagtgaccatctccgtggacaagtctgtgtccaccgcctacctgcagtggtcctctctgaagccttctgactccgccgtgtacttctgcgccaggcacgatgtgggctactgcaccgatagaacctgcgccaagtggcccgagtactttcagcattggggccagggcacactggtcaccgtttcttccgcctctacacagtcccctagcgtgttccctctgaccagatgctgcaagaacatcccctccaacgccacctctgtgaccctgggatgtctggccaccggctattttcccgagcctgtgatggtcacctgggacaccggatctctgaacggcaccaccatgacactgcctgccaccacactgaccctgtctggacactacgctaccatcagcctgctgacagtgtctggcgcttgggccaagcagatgttcacctgtagagtggctcacaccccttccagcaccgactgggtcgacaacaagaccttctccgtgtgctcccgggacttcacccctcctacagtgaagatcctgcagtcctcttgtgatggcggcggacacttcccacctacaatccagctgctgtgcctggtgtctggctatacccctggcaccatcaacatcacctggctggaagatggacaagtgatggacgtggacctgagcaccgccagcacaacacaagaaggcgagctggcttctacccagtccgagctgacactgtcccagaagcactggctgtccgaccggacctacacctgtcaagtgacctaccagggccacaccttcgaggactccaccaagaagtgcgccgacagcaatcctagaggcgtgtccgcttacctgagccggccttctcctttcgacctgttcatccggaagtctcccaccatcacatgtctggtggtggatctggccccttccaagggcaccgtgaatctgacctggtctagagcctctggcaagcccgtgaaccacagcacccggaaagaggaaaagcagcgcaacggcaccctgaccgtgacctctacactgccagtgggcaccagagattggatcgagggcgagacataccagtgcagagtgacccatcctcatctgcctcgggctctgatgcggtctaccaccaagacctctggacctagagctgcccctgaggtgtacgcctttgctacacctgagtggcctggctccagagacaagagaaccctggcctgcctgatccagaacttcatgcccgaggacatcagcgtgcagtggctgcacaatgaggtgcagctgcctgacgctcggcactctacaacccagcctagaaagaccaaaggcagcggcttcttcgtgttctctcggctggaagtgaccagagccgagtgggagcagaaggacgagttcatctgcagagccgtgcacgaggctgcctctccatctcagacagttcagagggccgtgtctgtgaaccccggcaagtaatgaattc

SEQ ID NO: 9 - DNA, 1753 bp, Artificial Sequence
  homo sapiens and Escherichia virus M13
aagcttgccgccaccatggaatggtcctgggtgttcctgttcttcctgtccgtgaccaccggcgtgcactctcaggttcagttggttcagtctggcgccgaagtgaagaagcctggcgagtccctgaagatctcctgcaaaggctccggctactccttcacctcctactggatcgcctgggtccgacagatgcctggaaagggcctcgagtacatgggcctgatctaccctggccagtccgataccaagtactcccctagcttccagggccaagtgaccatctccgtggacaagtctgtgtccaccgcctacctgcagtggtcctctctgaagccttctgactccgccgtgtacttctgcgccaggcacgatgtgggctactgcaccgatagaacctgcgccaagtggcccgagtactttcagcattggggccagggcacactggtcaccgtttcttccgcctctacacagtcccctagcgtgttccctctgaccagatgctgcaagaacatcccctccaacgccacctctgtgaccctgggatgtctggccaccggctattttcccgagcctgtgatggtcacctgggacaccggatctctgaacggcaccaccatgacactgcctgccaccacactgaccctgtctggacactacgctaccatcagcctgctgacagtgtctggcgcttgggccaagcagatgttcacctgtagagtggctcacaccccttccagcaccgactgggtcgacaacaagaccttctccgtgtgctcccgggacttcacccctcctacagtgaagatcctgcagtcctcttgtgatggcggcggacacttcccacctacaatccagctgctgtgcctggtgtctggctatacccctggcaccatcaacatcacctggctggaagatggacaagtgatggacgtggacctgagcaccgccagcacaacacaagaaggcgagctggcttctacccagtccgagctgacactgtcccagaagcactggctgtccgaccggacctacacctgtcaagtgacctaccagggccacaccttcgaggactccaccaagaagtgcgccgacagcaatcctagaggcgtgtccgcttacctgagccggccttctcctttcgacctgttcatccggaagtctcccaccatcacatgtctggtggtggatctggccccttccaagggcaccgtgaatctgacctggtctagagcctctggcaagcccgtgaaccacagcacccggaaagaggaaaagcagcgcaacggcaccctgaccgtgacctctacactgccagtgggcaccagagattggatcgagggcgagacataccagtgcagagtgacccatcctcatctgcctcgggctctgatgcggtctaccaccaagacctctggacctagagctgcccctgaggtgtacgcctttgctacacctgagtggcctggctccagagacaagagaaccctggcctgcctgatccagaacttcatgcccgaggacatcagcgtgcagtggctgcacaatgaggtgcagctgcctgacgctcggcactctacaacccagcctagaaagaccaaaggcagcggcttcttcgtgttctctcggctggaagtgaccagagccgagtgggagcagaaggacgagttcatctgcagagccgtgcacgaggctgcctctccatctcagacagttcagagggccgtgtctgtgaaccccggcaagtaatgaattc

SEQ ID NO: 10 - DNA, 1753 bp, Artificial Sequence
  homo sapiens and Escherichia virus M13
aagcttgccgccaccatggaatggtcctgggtgttcctgttcttcctgtccgtgaccaccggcgtgcactctcaggttcagttggttcagtctggcgccgaagtgaagaagcctggcgagtccctgaagatctcctgcaaaggctccggctactccttcacctcctactggatcgcctgggtccgacagatgcctggaaagggcctcgagtacatgggcctgatctaccctggctcctccgataccaagtactcccctagcttccagggccaagtgaccatctccgtggacaagtctgtgtccaccgcctacctgcagtggtcctctctgaagccttctgactccgccgtgtacttctgcgccaggcacgatgtgggctactgcaccgatagaacctgcgccaagtggcccgagtactttcagcattggggccagggcacactggtcaccgtttcttccgcctctacacagtcccctagcgtgttccctctgaccagatgctgcaagaacatcccctccaacgccacctctgtgaccctgggatgtctggccaccggctattttcccgagcctgtgatggtcacctgggacaccggatctctgaacggcaccaccatgacactgcctgccaccacactgaccctgtctggacactacgctaccatcagcctgctgacagtgtctggcgcttgggccaagcagatgttcacctgtagagtggctcacaccccttccagcaccgactgggtcgacaacaagaccttctccgtgtgctcccgggacttcacccctcctacagtgaagatcctgcagtcctcttgtgatggcggcggacacttcccacctacaatccagctgctgtgcctggtgtctggctatacccctggcaccatcaacatcacctggctggaagatggacaagtgatggacgtggacctgagcaccgccagcacaacacaagaaggcgagctggcttctacccagtccgagctgacactgtcccagaagcactggctgtccgaccggacctacacctgtcaagtgacctaccagggccacaccttcgaggactccaccaagaagtgcgccgacagcaatcctagaggcgtgtccgcttacctgagccggccttctcctttcgacctgttcatccggaagtctcccaccatcacatgtctggtggtggatctggccccttccaagggcaccgtgaatctgacctggtctagagcctctggcaagcccgtgaaccacagcacccggaaagaggaaaagcagcgcaacggcaccctgaccgtgacctctacactgccagtgggcaccagagattggatcgagggcgagacataccagtgcagagtgacccatcctcatctgcctcgggctctgatgcggtctaccaccaagacctctggacctagagctgcccctgaggtgtacgcctttgctacacctgagtggcctggctccagagacaagagaaccctggcctgcctgatccagaacttcatgcccgaggacatcagcgtgcagtggctgcacaatgaggtgcagctgcctgacgctcggcactctacaacccagcctagaaagaccaaaggcagcggcttcttcgtgttctctcggctggaagtgaccagagccgagtgggagcagaaggacgagttcatctgcagagccgtgcacgaggctgcctctccatctcagacagttcagagggccgtgtctgtgaaccccggcaagtaatgaattc

SEQ ID NO: 11 - DNA, 1753 bp, Artificial Sequence
  homo sapiens and Escherichia virus M13
aagcttgccgccaccatggaatggtcctgggtgttcctgttcttcctgtccgtgaccaccggcgtgcactctcaggttcagttggttcagtctggcgccgaagtgaagaagcctggcgagtccctgaagatctcctgcaaaggctccggctactccttcacctcctactggatcgcctgggtccgacagatgcctggaaagggcctcgagtacatgggcctgatctaccctggcgacaccgataccaagtacagccccagctttcagggccaagtgaccatctccgtggacaagtctgtgtccaccgcctacctgcagtggtcctctctgaagccttctgactccgccgtgtacttctgcgccaggcacgatgtgggctactgcaccgatagaacctgcgccaagtggcccgagtactttcagcattggggccagggcacactggtcaccgtttcttccgcctctacacagtcccctagcgtgttccctctgaccagatgctgcaagaacatcccctccaacgccacctctgtgaccctgggatgtctggccaccggctattttcccgagcctgtgatggtcacctgggacaccggatctctgaacggcaccaccatgacactgcctgccaccacactgaccctgtctggacactacgctaccatcagcctgctgacagtgtctggcgcttgggccaagcagatgttcacctgtagagtggctcacaccccttccagcaccgactgggtcgacaacaagaccttctccgtgtgctcccgggacttcacccctcctacagtgaagatcctgcagtcctcttgtgatggcggcggacacttcccacctacaatccagctgctgtgcctggtgtctggctatacccctggcaccatcaacatcacctggctggaagatggacaagtgatggacgtggacctgagcaccgccagcacaacacaagaaggcgagctggcttctacccagtccgagctgacactgtcccagaagcactggctgtccgaccggacctacacctgtcaagtgacctaccagggccacaccttcgaggactccaccaagaagtgcgccgacagcaatcctagaggcgtgtccgcttacctgagccggccttctcctttcgacctgttcatccggaagtctcccaccatcacatgtctggtggtggatctggccccttccaagggcaccgtgaatctgacctggtctagagcctctggcaagcccgtgaaccacagcacccggaaagaggaaaagcagcgcaacggcaccctgaccgtgacctctacactgccagtgggcaccagagattggatcgagggcgagacataccagtgcagagtgacccatcctcatctgcctcgggctctgatgcggtctaccaccaagacctctggacctagagctgcccctgaggtgtacgcctttgctacacctgagtggcctggctccagagacaagagaaccctggcctgcctgatccagaacttcatgcccgaggacatcagcgtgcagtggctgcacaatgaggtgcagctgcctgacgctcggcactctacaacccagcctagaaagaccaaaggcagcggcttcttcgtgttctctcggctggaagtgaccagagccgagtgggagcagaaggacgagttcatctgcagagccgtgcacgaggctgcctctccatctcagacagttcagagggccgtgtctgtgaaccccggcaagtaatgaattc

SEQ ID NO: 12 - DNA, 1753 bp, Artificial Sequence
  homo sapiens and Escherichia virus M13
aagcttgccgccaccatggaatggtcctgggtgttcctgttcttcctgtccgtgaccaccggcgtgcactctcaggttcagttggttcagtctggcgccgaagtgaagaagcctggcgagtccctgaagatctcctgcaaaggctccggctactccttcacctcctactggatcgcctgggtccgacagatgcctggaaagggcctcgagtacatgggcctgatctaccctggcgacatcgataccaagtacagccccagcttccagggccaagtgaccatctccgtggacaagtctgtgtccaccgcctacctgcagtggtcctctctgaagccttctgactccgccgtgtacttctgcgccaggcacgatgtgggctactgcaccgatagaacctgcgccaagtggcccgagtactttcagcattggggccagggcacactggtcaccgtttcttccgcctctacacagtcccctagcgtgttccctctgaccagatgctgcaagaacatcccctccaacgccacctctgtgaccctgggatgtctggccaccggctattttcccgagcctgtgatggtcacctgggacaccggatctctgaacggcaccaccatgacactgcctgccaccacactgaccctgtctggacactacgctaccatcagcctgctgacagtgtctggcgcttgggccaagcagatgttcacctgtagagtggctcacaccccttccagcaccgactgggtcgacaacaagaccttctccgtgtgctcccgggacttcacccctcctacagtgaagatcctgcagtcctcttgtgatggcggcggacacttcccacctacaatccagctgctgtgcctggtgtctggctatacccctggcaccatcaacatcacctggctggaagatggacaagtgatggacgtggacctgagcaccgccagcacaacacaagaaggcgagctggcttctacccagtccgagctgacactgtcccagaagcactggctgtccgaccggacctacacctgtcaagtgacctaccagggccacaccttcgaggactccaccaagaagtgcgccgacagcaatcctagaggcgtgtccgcttacctgagccggccttctcctttcgacctgttcatccggaagtctcccaccatcacatgtctggtggtggatctggccccttccaagggcaccgtgaatctgacctggtctagagcctctggcaagcccgtgaaccacagcacccggaaagaggaaaagcagcgcaacggcaccctgaccgtgacctctacactgccagtgggcaccagagattggatcgagggcgagacataccagtgcagagtgacccatcctcatctgcctcgggctctgatgcggtctaccaccaagacctctggacctagagctgcccctgaggtgtacgcctttgctacacctgagtggcctggctccagagacaagagaaccctggcctgcctgatccagaacttcatgcccgaggacatcagcgtgcagtggctgcacaatgaggtgcagctgcctgacgctcggcactctacaacccagcctagaaagaccaaaggcagcggcttcttcgtgttctctcggctggaagtgaccagagccgagtgggagcagaaggacgagttcatctgcagagccgtgcacgaggctgcctctccatctcagacagttcagagggccgtgtctgtgaaccccggcaagtaatgaattc

SEQ ID NO: 13 - protein, 581 aa, Artificial Sequence
  homo sapiens and Escherichia virus M13
KLAATMEWSWVFLFFLSVTTGVHSQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 14 - protein, 126 aa, Artificial Sequence
  homo sapiens and Escherichia virus M13
VQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTV

SEQ ID NO: 15 - protein, 24 aa, Artificial Sequence
  homo sapiens and Escherichia virus M13
VQLVQSGAEVKKPGESLKISCKGS

SEQ ID NO: 16 - protein, 17 aa, Artificial Sequence
  homo sapiens and Escherichia virus M13
IAWVRQMPGKGLEYMGL

SEQ ID NO: 17 - protein, 38 aa, Artificial Sequence
  homo sapiens and Escherichia virus M13
KYSPSFQGQVTISVDKSVSTAYLQWSSLKPSDSAVYFC

SEQ ID NO: 18 - protein, 9 aa, Artificial Sequence
  homo sapiens and Escherichia virus M13
WGQGTLVTV

SEQ ID NO: 19 - protein, 8 aa, Artificial Sequence
  homo sapiens and Escherichia virus M13
GYSFTSYW

SEQ ID NO: 20 - protein, 8 aa, Artificial Sequence
  homo sapiens and Escherichia virus M13
IYPGDSDT

SEQ ID NO: 21 - protein, 22 aa, Artificial Sequence
  homo sapiens and Escherichia virus M13
ARHDVGYCTDRTCAKWPEYFQH

SEQ ID NO: 22 - protein, 581 aa, Artificial Sequence
  homo sapiens and Escherichia virus M13
KLAATMEWSWVFLFFLSVTTGVHSQVQLVQSGAEVKKPGESLKISCKGSGYSFTSYWIAWVRQMPGKGLEYMGLIYPGESDTKYSPSFQGQVTISVDKSVSTAYLQWSSLKPSDSAVYFCARHDVGYCTDRTCAKWPEYFQHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 23 - protein, 126 aa, Artificial Sequence
  homo sapiens and Escherichia virus M13
VQLVQSGAEVKKPGESLKISCKGSGYSFTSYWIAWVRQMPGKGLEYMGLIYPGESDTKYSPSFQGQVTISVDKSVSTAYLQWSSLKPSDSAVYFCARHDVGYCTDRTCAKWPEYFQHWGQGTLVTV

SEQ ID NO: 24 - protein, 24 aa, Artificial Sequence
  homo sapiens and Escherichia virus M13
VQLVQSGAEVKKPGESLKISCKGS

SEQ ID NO: 25 - protein, 17 aa, Artificial Sequence
  homo sapiens and Escherichia virus M13
IAWVRQMPGKGLEYMGL

SEQ ID NO: 26 - protein, 38 aa, Artificial Sequence; identical to SEQ ID NO: 17
  homo sapiens and Escherichia virus M13

SEQ ID NO: 27 - protein, 12 aa, Artificial Sequence
  homo sapiens and Escherichia virus M13
FQHWGQGTLVTV

SEQ ID NO: 28 - protein, 8 aa, Artificial Sequence
  homo sapiens and Escherichia virus M13
GYSFTSYW

SEQ ID NO: 29 - protein, 8 aa, Artificial Sequence
  homo sapiens and Escherichia virus M13
IYPGESDT

SEQ ID NO: 30 - protein, 19 aa, Artificial Sequence
  homo sapiens and Escherichia virus M13
ARHDVGYCTDRTCAKWPEY

SEQ ID NO: 31 - protein, 581 aa, Artificial Sequence
  homo sapiens and Escherichia virus M13
KLAATMEWSWVFLFFLSVTTGVHSQVQLVQSGAEVKKPGESLKISCKGSGYSFTSYWIAWVRQMPGKGLEYMGLIYPGQSDTKYSPSFQGQVTISVDKSVSTAYLQWSSLKPSDSAVYFCARHDVGYCTDRTCAKWPEYFQHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 32 - protein, 126 aa, Artificial Sequence
  homo sapiens and Escherichia virus M13
VQLVQSGAEVKKPGESLKISCKGSGYSFTSYWIAWVRQMPGKGLEYMGLIYPGQSDTKYSPSFQGQVTISVDKSVSTAYLQWSSLKPSDSAVYFCARHDVGYCTDRTCAKWPEYFQHWGQGTLVTV

SEQ ID NO: 33 - protein, 24 aa, Artificial Sequence
  homo sapiens and Escherichia virus M13
VQLVQSGAEVKKPGESLKISCKGS

SEQ ID NO: 34 - protein, 17 aa, Artificial Sequence
  homo sapiens and Escherichia virus M13
IAWVRQMPGKGLEYMGL

SEQ ID NO: 35 - protein, 38 aa, Artificial Sequence; identical to SEQ ID NO: 17
  homo sapiens and Escherichia virus M13

SEQ ID NO: 36 - protein, 9 aa, Artificial Sequence
  homo sapiens and Escherichia virus M13
WGQGTLVTV

SEQ ID NO: 37 - protein, 8 aa, Artificial Sequence
  homo sapiens and Escherichia virus M13
GYSFTSYW

SEQ ID NO: 38 - protein, 8 aa, Artificial Sequence
  homo sapiens and Escherichia virus M13
IYPGQSDT

SEQ ID NO: 39 - protein, 22 aa, Artificial Sequence
  homo sapiens and Escherichia virus M13
ARHDVGYCTDRTCAKWPEYFQH

SEQ ID NO: 40 - protein, 581 aa, Artificial Sequence
  homo sapiens and Escherichia virus M13
KLAATMEWSWVFLFFLSVTTGVHSQVQLVQSGAEVKKPGESLKISCKGSGYSFTSYWIAWVRQMPGKGLEYMGLIYPGSSDTKYSPSFQGQVTISVDKSVSTAYLQWSSLKPSDSAVYFCARHDVGYCTDRTCAKWPEYFQHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 41 - protein, 127 aa, Artificial Sequence
  homo sapiens and Escherichia virus M13
QVQLVQSGAEVKKPGESLKISCKGSGYSFTSYWIAWVRQMPGKGLEYMGLIYPGSSDTKYSPSFQGQVTISVDKSVSTAYLQWSSLKPSDSAVYFCARHDVGYCTDRTCAKWPEYFQHWGQGTLVTV

SEQ ID NO: 42 - protein, 25 aa, Artificial Sequence
  homo sapiens and Escherichia virus M13
QVQLVQSGAEVKKPGESLKISCKGS

SEQ ID NO: 43 - protein, 17 aa, Artificial Sequence
  homo sapiens and Escherichia virus M13
IAWVRQMPGKGLEYMGL

SEQ ID NO: 44 - protein, 38 aa, Artificial Sequence; identical to SEQ ID NO: 17
  homo sapiens and Escherichia virus M13

SEQ ID NO: 45 - protein, 9 aa, Artificial Sequence
  homo sapiens and Escherichia virus M13
WGQGTLVTV

SEQ ID NO: 46 - protein, 8 aa, Artificial Sequence
  homo sapiens and Escherichia virus M13
GYSFTSYW

SEQ ID NO: 47 - protein, 8 aa, Artificial Sequence
  homo sapiens and Escherichia virus M13
IYPGSSDT

SEQ ID NO: 48 - protein, 22 aa, Artificial Sequence
  homo sapiens and Escherichia virus M13
ARHDVGYCTDRTCAKWPEYFQH

SEQ ID NO: 49 - protein, 581 aa, Artificial Sequence
  homo sapiens and Escherichia virus M13
KLAATMEWSWVFLFFLSVTTGVHSQVQLVQSGAEVKKPGESLKISCKGSGYSFTSYWIAWVRQMPGKGLEYMGLIYPGDTDTKYSPSFQGQVTISVDKSVSTAYLQWSSLKPSDSAVYFCARHDVGYCTDRTCAKWPEYFQHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 50 - protein, 126 aa, Artificial Sequence
  homo sapiens and Escherichia virus M13
VQLVQSGAEVKKPGESLKISCKGSGYSFTSYWIAWVRQMPGKGLEYMGLIYPGDTDTKYSPSFQGQVTISVDKSVSTAYLQWSSLKPSDSAVYFCARHDVGYCTDRTCAKWPEYFQHWGQGTLVTV

SEQ ID NO: 51 - protein, 24 aa, Artificial Sequence
  homo sapiens and Escherichia virus M13
VQLVQSGAEVKKPGESLKISCKGS

SEQ ID NO: 52 - protein, 17 aa, Artificial Sequence
  homo sapiens and Escherichia virus M13
IAWVRQMPGKGLEYMGL

SEQ ID NO: 53 - protein, 38 aa, Artificial Sequence; identical to SEQ ID NO: 17
  homo sapiens and Escherichia virus M13

SEQ ID NO: 54 - protein, 9 aa, Artificial Sequence
  homo sapiens and Escherichia virus M13
WGQGTLVTV

SEQ ID NO: 55 - protein, 8 aa, Artificial Sequence
  homo sapiens and Escherichia virus M13
GYSFTSYW

SEQ ID NO: 56 - protein, 8 aa, Artificial Sequence
  homo sapiens and Escherichia virus M13
IYPGDTDT

SEQ ID NO: 57 - protein, 22 aa, Artificial Sequence
  homo sapiens and Escherichia virus M13
ARHDVGYCTDRTCAKWPEYFQH

SEQ ID NO: 58 - protein, 581 aa, Artificial Sequence
  homo sapiens and Escherichia virus M13
KLAATMEWSWVFLFFLSVTTGVHSQVQLVQSGAEVKKPGESLKISCKGSGYSFTSYWIAWVRQMPGKGLEYMGLIYPGDIDTKYSPSFQGQVTISVDKSVSTAYLQWSSLKPSDSAVYFCARHDVGYCTDRTCAKWPEYFQHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 59 - protein, 126 aa, Artificial Sequence
  homo sapiens and Escherichia virus M13
VQLVQSGAEVKKPGESLKISCKGSGYSFTSYWIAWVRQMPGKGLEYMGLIYPGDIDTKYSPSFQGQVTISVDKSVSTAYLQWSSLKPSDSAVYFCARHDVGYCTDRTCAKWPEYFQHWGQGTLVTV

SEQ ID NO: 60 - protein, 24 aa, Artificial Sequence
  homo sapiens and Escherichia virus M13
VQLVQSGAEVKKPGESLKISCKGS

SEQ ID NO: 61 - protein, 17 aa, Artificial Sequence
  homo sapiens and Escherichia virus M13
IAWVRQMPGKGLEYMGL

SEQ ID NO: 62 - protein, 38 aa, Artificial Sequence; identical to SEQ ID NO: 17
  homo sapiens and Escherichia virus M13

SEQ ID NO: 63 - protein, 9 aa, Artificial Sequence
  homo sapiens and Escherichia virus M13
WGQGTLVTV

SEQ ID NO: 64 - protein, 8 aa, Artificial Sequence
  homo sapiens and Escherichia virus M13
GYSFTSYW

SEQ ID NO: 65 - protein, 8 aa, Artificial Sequence
  homo sapiens and Escherichia virus M13
IYPGDIDT

SEQ ID NO: 66 - protein, 22 aa, Artificial Sequence
  homo sapiens and Escherichia virus M13
ARHDVGYCTDRTCAKWPEYFQH

SEQ ID NO: 67 - DNA, 733 bp, Artificial Sequence
  homo sapiens and Escherichia virus M13
aagcttgccgccaccatgtctgtgcctacacaggttctgggactgctgctgctgtggctgaccgatgctagatgccagtccgtgctgacccagcctccttctgtttctgctgcccctggccagaaagtgaccatctcttgctccggctcctcctccaacatcggcaacaactacgtgtcctggtatcagcagctgcccggcacagctcccaaactgctgatctacgaccacaccaacagacctgccggcgtgcccgatagattctccggctctaagtctggcacctctgccagcctggccatctccggctttagatctgaggacgaggccgactactactgcgcctcttgggactataccctgtccggctgggttttcggcggaggcacaaaagtgaccgtcctaggtcaacccaaggccgctcccagcgtgaccctgttcccccccagcagcgaggagctgcaggccaacaaggccaccctggtgtgtctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgaccccagagcagtggaagagccacaggagctacagctgccaggtcacccacgagggcagcaccgtggaaaagaccgtggccccaaccgagtgctcctgatgaattc

SEQ ID NO: 68 - DNA, 733 bp, Artificial Sequence; identical to SEQ ID NO: 67
  homo sapiens and Escherichia virus M13

SEQ ID NO: 69 - protein, 733 aa, Artificial Sequence
  homo sapiens and Escherichia virus M13
AAGCTTGCCGCCACCATGTCTGTGCCTACACAGGTTCTGGGACTGCTGCTGCTGTGGCTGACCGATGCTAGATGCCAGTCCGTGCTGACCCAGCCTCCTTCTGTTTCTGCTGCCCCTGGCCAGAAAGTGACCATCTCTTGCTCCGGCTCCTCCTCCAACATCGGCAACAACTACGTGTCCTGGTATCAGCAGCTGCCCGGCACAGCTCCCAAACTGCTGATCTACGACCACACCAACAGACCTGCCGGCGTGCCCGATAGATTCTCCGGCTCTAAGTCTGGCACCTCTGCCAGCCTGGCCATCTCCGGCTTTAGATCTGAGGACGAGGCCGACTACTACTGCGCCTCTTGGGACTATACCCTGTCCGGCTGGGTTTTCGGCGGAGGCACAAAAGTGACCGTCCTAGGTCAACCCAAGGCCGCTCCCAGCGTGACCCTGTTCCCCCCCAGCAGCGAGGAGCTGCAGGCCAACAAGGCCACCCTGGTGTGTCTGATCAGCGACTTCTACCCAGGCGCCGTGACCGTGGCCTGGAAGGCCGACAGCAGCCCCGTGAAGGCCGGCGTGGAGACCACCACCCCCAGCAAGCAGAGCAACAACAAGTACGCCGCCAGCAGCTACCTGAGCCTGACCCCAGAGCAGTGGAAGAGCCACAGGAGCTACAGCTGCCAGGTCACCCACGAGGGCAGCACCGTGGAAAAGACCGTGGCCCCAACCGAGTGCTCCTGATGAATTC

SEQ ID NO: 70 - DNA, 733 bp, Artificial Sequence; identical to SEQ ID NO: 67
  homo sapiens and Escherichia virus M13

SEQ ID NO: 71 - DNA, 733 bp, Artificial Sequence; identical to SEQ ID NO: 67
  homo sapiens and Escherichia virus M13

SEQ ID NO: 72 - DNA, 733 bp, Artificial Sequence; identical to SEQ ID NO: 67
  homo sapiens and Escherichia virus M13

SEQ ID NO: 73 - protein, 241 aa, Artificial Sequence
  homo sapiens and Escherichia virus M13
KLAATMSVPTQVLGLLLLWLTDARCQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 74 - protein, 110 aa, Artificial Sequence
  homo sapiens and Escherichia virus M13
QSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKVTVL

SEQ ID NO: 75 - protein, 25 aa, Artificial Sequence
  homo sapiens and Escherichia virus M13
QSVLTQPPSVSAAPGQKVTISCSGS

SEQ ID NO: 76 - protein, 17 aa, Artificial Sequence
  homo sapiens and Escherichia virus M13
VSWYQQLPGTAPKLLIY

SEQ ID NO: 77 - protein, 36 aa, Artificial Sequence
  homo sapiens and Escherichia virus M13
NRPAGVPDRFSGSKSGTSASLAISGFRSEDEADYYC

SEQ ID NO: 78 - protein, 10 aa, Artificial Sequence
  homo sapiens and Escherichia virus M13
FGGGTKVTVL

SEQ ID NO: 79 - protein, 8 aa, Artificial Sequence
  homo sapiens and Escherichia virus M13
SSNIGNNY

SEQ ID NO: 80 - protein, 3 aa, Artificial Sequence
  homo sapiens and Escherichia virus M13
DHT

SEQ ID NO: 81 - protein, 11 aa, Artificial Sequence
  homo sapiens and Escherichia virus M13
ASWDYTLSGWV

SEQ ID NO: 82 - protein, 241 aa, Artificial Sequence; identical to SEQ ID NO: 73
  homo sapiens and Escherichia virus M13

SEQ ID NO: 83 - protein, 110 aa, Artificial Sequence; identical to SEQ ID NO: 74
  homo sapiens and Escherichia virus M13

SEQ ID NO: 84 - protein, 25 aa, Artificial Sequence
  homo sapiens and Escherichia virus M13
QSVLTQPPSVSAAPGQKVTISCSGS

SEQ ID NO: 85 - protein, 16 aa, Artificial Sequence
  homo sapiens and Escherichia virus M13
SWYQQLPGTAPKLLIY

SEQ ID NO: 86 - protein, 36 aa, Artificial Sequence; identical to SEQ ID NO: 77
  homo sapiens and Escherichia virus M13

SEQ ID NO: 87 - protein, 10 aa, Artificial Sequence
  homo sapiens and Escherichia virus M13
FGGGTKVTVL

SEQ ID NO: 88 - protein, 9 aa, Artificial Sequence
  homo sapiens and Escherichia virus M13
SSNIGNNYV

SEQ ID NO: 89 - protein, 3 aa, Artificial Sequence
  homo sapiens and Escherichia virus M13
DHT

SEQ ID NO: 90 - protein, 11 aa, Artificial Sequence
  homo sapiens and Escherichia virus M13
ASWDYTLSGWV

SEQ ID NO: 91 - protein, 241 aa, Artificial Sequence; identical to SEQ ID NO: 73
  homo sapiens and Escherichia virus M13

SEQ ID NO: 92 - protein, 110 aa, Artificial Sequence; identical to SEQ ID NO: 74
  homo sapiens and Escherichia virus M13

SEQ ID NO: 93 - protein, 25 aa, Artificial Sequence
  homo sapiens and Escherichia virus M13
QSVLTQPPSVSAAPGQKVTISCSGS

SEQ ID NO: 94 - protein, 17 aa, Artificial Sequence
  homo sapiens and Escherichia virus M13
VSWYQQLPGTAPKLLIY

SEQ ID NO: 95 - protein, 36 aa, Artificial Sequence; identical to SEQ ID NO: 77
  homo sapiens and Escherichia virus M13

SEQ ID NO: 96 - protein, 10 aa, Artificial Sequence
  homo sapiens and Escherichia virus M13
FGGGTKVTVL

SEQ ID NO: 97 - protein, 8 aa, Artificial Sequence
  homo sapiens and Escherichia virus M13
SSNIGNNY

SEQ ID NO: 98 - protein, 3 aa, Artificial Sequence
  homo sapiens and Escherichia virus M13
DHT

SEQ ID NO: 99 - protein, 11 aa, Artificial Sequence
  homo sapiens and Escherichia virus M13
ASWDYTLSGWV

SEQ ID NO: 100 - protein, 241 aa, Artificial Sequence; identical to SEQ ID NO: 73
  homo sapiens and Escherichia virus M13

SEQ ID NO: 101 - protein, 110 aa, Artificial Sequence; identical to SEQ ID NO: 74
  homo sapiens and Escherichia virus M13

SEQ ID NO: 102 - protein, 25 aa, Artificial Sequence
  homo sapiens and Escherichia virus M13
QSVLTQPPSVSAAPGQKVTISCSGS

SEQ ID NO: 103 - protein, 17 aa, Artificial Sequence
  homo sapiens and Escherichia virus M13
VSWYQQLPGTAPKLLIY

SEQ ID NO: 104 - protein, 36 aa, Artificial Sequence; identical to SEQ ID NO: 77
  homo sapiens and Escherichia virus M13

SEQ ID NO: 105 - protein, 10 aa, Artificial Sequence
  homo sapiens and Escherichia virus M13
FGGGTKVTVL

SEQ ID NO: 106 - protein, 8 aa, Artificial Sequence
  homo sapiens and Escherichia virus M13
SSNIGNNY

SEQ ID NO: 107 - protein, 3 aa, Artificial Sequence
  homo sapiens and Escherichia virus M13
DHT

SEQ ID NO: 108 - protein, 11 aa, Artificial Sequence
  homo sapiens and Escherichia virus M13
ASWDYTLSGWV

SEQ ID NO: 109 - protein, 241 aa, Artificial Sequence; identical to SEQ ID NO: 73
  homo sapiens and Escherichia virus M13

SEQ ID NO: 110 - protein, 110 aa, Artificial Sequence; identical to SEQ ID NO: 74
  homo sapiens and Escherichia virus M13

SEQ ID NO: 111 - protein, 25 aa, Artificial Sequence
  homo sapiens and Escherichia virus M13
QSVLTQPPSVSAAPGQKVTISCSGS

SEQ ID NO: 112 - protein, 17 aa, Artificial Sequence
  homo sapiens and Escherichia virus M13
VSWYQQLPGTAPKLLIY

SEQ ID NO: 113 - protein, 36 aa, Artificial Sequence; identical to SEQ ID NO: 77
  homo sapiens and Escherichia virus M13

SEQ ID NO: 114 - protein, 10 aa, Artificial Sequence
  homo sapiens and Escherichia virus M13
FGGGTKVTVL

SEQ ID NO: 115 - protein, 8 aa, Artificial Sequence
  homo sapiens and Escherichia virus M13
SSNIGNNY

SEQ ID NO: 116 - protein, 3 aa, Artificial Sequence
  homo sapiens and Escherichia virus M13
DHT

SEQ ID NO: 117 - protein, 11 aa, Artificial Sequence
  homo sapiens and Escherichia virus M13
ASWDYTLSGWV

SEQ ID NO: 118 - protein, 241 aa, Artificial Sequence; identical to SEQ ID NO: 73
  homo sapiens and Escherichia virus M13

SEQ ID NO: 119 - protein, 110 aa, Artificial Sequence; identical to SEQ ID NO: 74
  homo sapiens and Escherichia virus M13

SEQ ID NO: 120 - protein, 25 aa, Artificial Sequence
  homo sapiens and Escherichia virus M13
QSVLTQPPSVSAAPGQKVTISCSGS

SEQ ID NO: 121 - protein, 17 aa, Artificial Sequence
  homo sapiens and Escherichia virus M13
VSWYQQLPGTAPKLLIY

SEQ ID NO: 122 - protein, 36 aa, Artificial Sequence; identical to SEQ ID NO: 77
  homo sapiens and Escherichia virus M13

SEQ ID NO: 123 - protein, 10 aa, Artificial Sequence
  homo sapiens and Escherichia virus M13
FGGGTKVTVL

SEQ ID NO: 124 - protein, 8 aa, Artificial Sequence
  homo sapiens and Escherichia virus M13
SSNIGNNY

SEQ ID NO: 125 - protein, 3 aa, Artificial Sequence
  homo sapiens and Escherichia virus M13
DHT

SEQ ID NO: 126 - protein, 11 aa, Artificial Sequence
  homo sapiens and Escherichia virus M13
ASWDYTLSGWV

(19) United States
(12) Patent Application Publication
(10) Pub. No.: US 2023/0070306 A1
(43) Pub. Date: Mar. 9, 2023

(54) MONOCLONAL ANTIBODIES AGAINST HER2/NEU AND USES THEREOF

(71) Applicant: ONCOQUEST PHARMACEUTICALS INC., Miryang-Si (KR)

(72) Inventors: Ragupathy Madiyalakan, Edmonton (CA); Thomas Woo, Edmonton (CA)

(21) Appl. No.: 17/406,171

(22) Filed: Aug. 19, 2021

(51) Int. Cl. C07K 16/32

(52) U.S. Cl. CPC C07K 16/32; C07K 2317/24; C07K 2317/31; C07K 2319/00

(57) ABSTRACT

The present document describes an antibody or an antigen-binding fragment comprising three variable heavy domain complementarity determining regions (CDR) (CDR H1, H2 and H3) that binds specifically to Her2/Neu. The present invention also relates to pharmaceutical compositions, nucleic acid molecule, vectors, cells comprising the nucleic acid vectors, and methods of treating Her2/Neu associated diseases.

### BACKGROUND

### (a) Field

The subject matter disclosed generally relates to monoclonal antibodies against Her2/Neu. More specifically, the subject matter relates to monoclonal antibodies against Her2/Neu that do not self-aggregate and methods of using the same.

### (b) Related Prior Art

Breast and ovarian cancer are two of the leading causes of cancer deaths among women and overexpression of the Her2/neu oncoprotein has been reported in patients affected with breast and ovarian cancers, and is associated with poor prognosis. Targeted therapy for Her2/neu expressing tumors may be targeted with monoclonal antibodies, such as antibodies comprising the variable regions of the scFv C6MH3-B1 specific for HER2/neu. However, such antibodies can suffer from low production yield, and could be further affected by numerous issues such as self-aggregation, oxidation, deamination and isomerization of amino acid chains. Ultimately, only small quantities of these antibodies can be prepared, which precludes large scale preparation for practical therapeutic application developments.

Therefore, there is a need for alternative monoclonal antibodies specific for HER2/neu that at least do not self-aggregate, improve their manufacturability and allow their preparation on a scale that allows their use as therapeutics.

### SUMMARY

According to an embodiment, there is provided an antibody or an antigen-binding fragment that binds specifically to Her2/Neu comprising three variable heavy domain complementarity determining regions (CDR) (CDR H1, H2 and H3) wherein the CDR H1, H2, and H3, comprise an amino acid sequence comprising:
CDR H1: GYSFTSYW (SEQ ID NO:1),
CDR H2: IYPGX₁X₂DT, where X₁ is D, E, or Q, X₂ is S, I, or T, and wherein when X₁ is D, X₂ is different than S (SEQ ID NO:2), and
CDR H3: ARHDVGYCTDRTCAKWPEY (SEQ ID NO:3), respectively.

The antibody or an antigen-binding fragment may comprise three variable light domain CDR (CDR L1, L2 and L3), wherein the CDR L1, L2, and L3 comprise an amino acid sequence comprising:
CDR L1: SSNIGNNY (SEQ ID NO:4),
CDR L2: DHT (SEQ ID NO:5), and
CDR L3: ASWDYTLSGVVV (SEQ ID NO:6), respectively.

In the antibody or antigen-binding fragment, X₁ may be D, X₂ may be I, or T.

In the antibody or antigen-binding fragment, wherein when X₁ is E, or Q, X₂ is S.

In the antibody or antigen-binding fragment, wherein X₁ may be E and X₂ may be S.

In the antibody or antigen-binding fragment, wherein X₁ may be Q and X₂ may be S.

In the antibody or antigen-binding fragment, wherein X₁ may be D and X₂ may be I.

In the antibody or antigen-binding fragment, wherein X₁ may be D and X₂ may be T.

The antibody or antigen-binding fragment may further comprise four variable heavy domain framework regions (HFR)(HFR 1, 2, 3 and 4), wherein said HFR 1, 2, 3, and 4 comprise an amino acid sequence comprising:

(SEQ ID NO: 15)
HFR 1: VQLVQSGAEVKKPGESLKISCKGS,
(SEQ ID NO: 16)
HFR 2: IAWVRQMPGKGLEYMGL,
(SEQ ID NO: 17)
HFR 3: KYSPSFQGQVTISVDKSVSTAYLQWSSLKPSDSAVYFC,
and
(SEQ ID NO: 18)
HFR 4: WGQGTLVTV.

The antibody or antigen-binding fragment may further comprise four variable light domain framework regions (LFR)(LFR 1, 2, 3 and 4), wherein said LFR 1, 2, 3, and 4 comprise an amino acid sequence comprising:

(SEQ ID NO: 84)
LFR 1: QSVLTQPPSVSAAPGQKVTISCSGS,
(SEQ ID NO: 85)
LFR 2: VSWYQQLPGTAPKLLIY,
(SEQ ID NO: 86)
LFR 3: NRPAGVPDRFSGSKSGTSASLAISGFRSEDEADYYC,
and
(SEQ ID NO: 87)
LFR 4: FGGGTKVTVL.

The antibody or antigen-binding fragment may comprise a variable heavy domain (V_{H}) comprising amino acid sequence comprising:

(SEQ ID NO: 23)
VQLVQSGAEVKKPGESLKISCKGSGYSFTSYWIAWVRQMPGKGLEYMGLI
YPGESDTKYSPSFQGQVTISVDKSVSTAYLQWSSLKPSDSAVYFCARHDV
GYCTDRTCAKWPEYFQHWGQGTLVTV.

The antibody or antigen binding fragment may comprise a variable light domain (V_{L}) comprising amino acid sequence comprising:

(SEQ ID NO: 83)
QSVLTQPPSVSAAPGQKVTISCSGSSSNIGNNYVSWYQQLPGTAPKLLIY
DHTNRPAGVPDRFSGSKSGTSASLAISGFRSEDEADYYCASWDYTLSGWV
FGGGTKVTVL.

The antibody or antigen-binding fragment may comprise a variable heavy domain (V_{H}) comprising amino acid sequence comprising:

(SEQ ID NO: 23)
VQLVQSGAEVKKPGESLKISCKGSGYSFTSYWIAWVRQMPGKGLEYMGLI
YPGESDTKYSPSFQGQVTISVDKSVSTAYLQWSSLKPSDSAVYFCARHDV
GYCTDRTCAKWPEYFQHWGQGTLVTV
and a variable light domain (V_{L}) comprising amino
acid sequence comprising:
(SEQ ID NO: 83)
QSVLTQPPSVSAAPGQKVTISCSGSSSNIGNNYVSWYQQLPGTAPKLLIY
DHTNRPAGVPDRFSGSKSGTSASLAISGFRSEDEADYYCASWDYTLSGWV
FGGGTKVTVL.

The antigen-binding fragment may be a single-domain antibody (sdAb), a fragment antigen binding (Fab), a single-chain variable fragment (scFv), or a single-chain fragment antigen binding (scFab).

The antibody or antigen-binding fragment may be an IgA, an IgD, an IgE, an IgG, or an IgM.

The antibody or antigen-binding fragment may be an IgE.

The antibody or antigen-binding fragment may be humanized or partially humanized.

According to another embodiment, there is provided a compound comprising the antibody or antigen-binding fragment of the present invention, and a functional moiety.

The antibody or antigen-binding fragment may be linked to the functional moiety via a peptide linker.

The antibody or antigen-binding fragment may be functionally linked to the functional moiety via the peptide linker.

The peptide linker may comprise about 3 to about 40 amino acid residues.

The peptide linker may comprise the amino acid sequence (GGGGS)ₙ or (GGGS)ₙ, wherein n 1.

The antibody or antigen-binding fragment may be fused to a second antibody or antigen-binding fragment operable to bind a target epitope.

The antibody or antigen-binding fragment may be linked to a peptide, a polypeptide, a protein, an enzyme, a second antibody, an antibody fragment, a second antigen-binding fragment or a combination of any two or more thereof; wherein each of the antibody or antigen-binding fragment thereof and the linked peptide, polypeptide, protein, enzyme, second antibody, antibody fragment, second antigen-binding fragment, or the combination of any two or more thereof is functional.

The antibody fragment may be a fragment crystallizable (Fc) region.

According to another embodiment, there is provided a composition comprising the antibody or antigen-binding fragment of the present invention or the compound of the present invention, and a pharmaceutically acceptable diluent, carrier or excipient.

According to another embodiment, there is provided a nucleic acid molecule encoding an antibody or antigen-binding fragment of the present invention, or encoding a compound of the present invention.

According to another embodiment, there is provided a vector comprising the nucleic acid molecule of the present invention operably linked to one or more regulatory elements to allow expression of the antibody or antigen-binding fragment the present invention, or the compound of the present invention in a host cell.

According to another embodiment, there is provided a cell comprising the vector of the present invention for expressing the antibody or antigen-binding fragment thereof of the present invention or the compound of the present invention.

According to another embodiment, there is provided a method of treating a Her2/Neu associated disease comprising: administering the antibody or antigen-binding fragment of the present invention, the compound of the present invention, the composition of the present invention, or a combination of any two or more thereof to a subject in need thereof.

The Her2/Neu associated disease may be a cancer.

The cancer may be ovarian cancer, breast cancer, stomach cancer, lung cancer, uterine cancer, salivary gland cancer, testicular germ cell cancer, bladder cancer, pancreatic cancer, and esophageal cancer.

According to another embodiment, there is provided a use of the antibody or antigen-binding fragment of the present invention, the compound of the present invention, the composition of the present invention, or a combination of any two or more thereof to a subject in need thereof in the treatment of a Her2/Neu associated disease.

The Her2/Neu associated disease may be a cancer.

The cancer may be ovarian cancer, breast cancer, stomach cancer, lung cancer, uterine cancer, salivary gland cancer, testicular germ cell cancer, bladder cancer, pancreatic cancer, and esophageal cancer.

According to another embodiment, there is provided an antibody or antigen-binding fragment of the present invention, the compound of the present invention, the composition of the present invention, or a combination of any two or more thereof to a subject in need thereof, for use in the treatment of a Her2/Neu associated disease.

The antibody or antigen-binding fragment, compound, composition, or combination of any two or more thereof of the present invention, wherein the Her2/Neu associated disease may be a cancer.

The antibody or antigen-binding fragment, compound, composition, or combination of any two or more thereof the present invention, wherein the cancer may be ovarian cancer, breast cancer, stomach cancer, lung cancer, uterine cancer, salivary gland cancer, testicular germ cell cancer, bladder cancer, pancreatic cancer, and esophageal cancer.

According to another embodiment, there is provided a use of the antibody or antigen-binding fragment of the present invention, the compound of the present invention, the composition of the present invention, or a combination of any two or more thereof to a subject in need thereof in the manufacture of a medicament for the treatment of a Her2/Neu associated disease.

The Her2/Neu associated disease may be a cancer.

The cancer may be ovarian cancer, breast cancer, stomach cancer, lung cancer, uterine cancer, salivary gland cancer, testicular germ cell cancer, bladder cancer, pancreatic cancer, and esophageal cancer.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1A shows an apparent affinity non-linear regression curve comparing the binding on D2F2/E2 cells of parental IgE C6MH3-B1 antibody versus variant 1. Each of the wild-type and Variant 1, Peak 1 (V1, P1) antibodies are used at dose −4 (0.0002 nM), −3 (0.002 nM), −2 (0.02 nM), −1 (0.2 nM), 0 (2 nM), 1 (20 nM), and 2 (200 nM).

FIG. 1B shows an apparent affinity non-linear regression curve comparing the binding on D2F2/E2 cells of parental IgE C6MH3-B1 antibody versus variant 2. Each of the wild-type and Variant 2, Peak 1 (V2, P1) antibodies are used at dose −4 (0.0002 nM), −3 (0.002 nM), −2 (0.02 nM), −1 (0.2 nM), 0 (2 nM), 1 (20 nM), and 2 (200 nM).

FIG. 1C shows an apparent affinity non-linear regression curve comparing the binding on D2F2/E2 cells of parental IgE C6MH3-B1 antibody versus variant 4. Each of the wild-type and Variant 4, Peak 1 (V4, P1) antibodies are used at dose −4 (0.0002 nM), −3 (0.002 nM), −2 (0.02 nM), −1 (0.2 nM), 0 (2 nM), 1 (20 nM), and 2 (200 nM).

FIG. 1D shows an apparent affinity non-linear regression curve comparing the binding on D2F2/E2 cells of parental IgE C6MH3-B1 antibody versus variant 5. Each of the wild-type and Variant 5, Peak 1 (V5, P1) antibodies are used at dose −4 (0.0001 nM), −3 (0.001 nM), −2 (0.01 nM), −1 (0.1 nM), 0 (1 nM), 1 (10 nM), and 2 (100 nM).

It will be noted that throughout the appended drawings, like features are identified by like reference numerals.

### DETAILED DESCRIPTION

The present invention is directed to a technology for binding specifically to Her2/Neu. In embodiments there is disclosed an antibody or an antigen-binding fragment that specifically binds to Her2/Neu, that comprises three variable heavy domain complementarity determining regions (CDR H1, CDR H2 and CDR H3).

The three variable heavy domain complementarity determining regions (CDR) (CDR H1, H2 and H3) may comprise the following amino acid sequences:

1) CDR H1:
(SEQ ID NO: 1)
GYSFTSYW,
2) CDR H2:
(SEQ ID NO: 2)
IYPGX₁X₂DT, where X₁ is D, E, or Q, X₂ is S, I,
or T, and wherein when X₁ is D, X₂ is different
than S,
and
3) CDR H3:
(SEQ ID NO: 3)
ARHDVGYCTDRTCAKWPEY.

According to an embodiment, the antibody or an antigen-binding fragment, may also comprise three variable light domain CDR (CDR L1, L2 and L3). The three variable light domain CDR (CDR L1, L2 and L3) may comprise the following amino acid sequences:

(SEQ ID NO: 4)
1) CDR L1: SSNIGNNY,
(SEQ ID NO: 5)
2) CDR L2: DHT,
and
(SEQ ID NO: 6)
3) CDR L3: ASWDYTLSGWV.

According to embodiments, the antibody or an antigen-binding fragment that specifically binds to Her2/Neu may comprise a CDR H2 wherein when X₁ is D, X₂ is I, or T (e.g. as per SEQ ID NOs: 65 and 56, respectively). According to other embodiments, the antibody or an antigen-binding fragment that specifically binds to Her2/Neu may comprise a CDR H2 wherein when X₁ is E, or Q, X₂ is S (e.g. as per SEQ ID NOs: 29, and 38, respectively). According to another embodiment, the antibody or an antigen-binding fragment that specifically binds to Her2/Neu may comprise a CDR H2 wherein X₁ is E and X₂ is S (e.g. as per SEQ ID NO: 29). According to another embodiment, the antibody or an antigen-binding fragment that specifically binds to Her2/Neu may comprise a CDR H2 wherein X₁ is Q and X₂ is S (e.g. as per SEQ ID NO: 38). According to another embodiment, the antibody or an antigen-binding fragment that specifically binds to Her2/Neu may comprise a CDR H2 wherein X₁ is D and X₂ is I (e.g. as per SEQ ID NO: 65). According to another embodiment, the antibody or an antigen-binding fragment that specifically binds to Her2/Neu may comprise a CDR H2 wherein X₁ is D and X₂ is T (e.g. as per SEQ ID NO: 56). (See Table A, below)

According to other embodiments, the antibody or antigen-binding fragment of the present invention may be an antibody comprising four variable heavy domain framework regions (HFR 1 to HFR4) and three variable heavy domain complementarity determining regions (CDR H1, CDR H2 and CDR H3), comprising amino acid sequence comprising:

(SEQ ID NO: 23)
VQLVQSGAEVKKPGESLKISCKGSGYSFTSYWIAWVRQMPGKGLEYMGLI
YPGESDTKYSPSFQGQVTISVDKSVSTAYLQWSSLKPSDSAVYFCARHDV
GYCTDRTCAKWPEYFQHWGQGTLVTV,
(SEQ ID NO: 32)
VQLVQSGAEVKKPGESLKISCKGSGYSFTSYWIAWVRQMPGKGLEYMGLI
YPGQSDTKYSPSFQGQVTISVDKSVSTAYLQWSSLKPSDSAVYFCARHDV
GYCTDRTCAKWPEYFQHWGQGTLVTV,
(SEQ ID NO: 41)
QVQLVQSGAEVKKPGESLKISCKGSGYSFTSYWIAWVRQMPGKGLEYMGL
IYPGSSDTKYSPSFQGQVTISVDKSVSTAYLQWSSLKPSDSAVYFCARHD
VGYCTDRTCAKWPEYFQHWGQGTLVTV,
(SEQ ID NO: 50)
VQLVQSGAEVKKPGESLKISCKGSGYSFTSYWIAWVRQMPGKGLEYMGLI
YPGDTDTKYSPSFQGQVTISVDKSVSTAYLQWSSLKPSDSAVYFCARHDV
GYCTDRTCAKWPEYFQHWGQGTLVTV,
or
(SEQ ID NO: 59)
VQLVQSGAEVKKPGESLKISCKGSGYSFTSYWIAWVRQMPGKGLEYMGLI
YPGDIDTKYSPSFQGQVTISVDKSVSTAYLQWSSLKPSDSAVYFCARHDV
GYCTDRTCAKWPEYFQHWGQGTLVTV,

that binds specifically to a Her2/Neu. It is believed to be operable to neutralize Her2/Neu, including, but not limited to a Her2/Neu family member from human, monkey, rat, or mouse.

According to other embodiments, the antibody or antigen-binding fragment of the present invention may be an antibody comprising variable heavy domain (V_{H}) comprising amino acid sequences comprising according to SEQ ID NOs: 22, 31, 49 or 58, respectively, that binds specifically to a Her2/Neu. It is believed to be operable to neutralize Her2/Neu, including, but not limited to a Her2/Neu family member from human, monkey, rat, or mouse.

According to other embodiments, the antibody or antigen-binding fragment of the present invention may be an antibody comprising four variable light domain framework regions (LFR1 to LFR4) and three variable light domain complementarity determining regions (CDR L1, CDR L2 and CDR L3), comprising amino acid sequence comprising:

QSVLTQPPSVSAAPGQKVTISCSGSSSNIGNNYVSWYQQLPGTAPKWYDHTNRPAGVPDRFSGS KSGTSASLAISGFRSEDEADYYCASWDYTLSGWVFGGGTKVTVL (as per each of SEQ ID NOs: 83, 92, 101, 110 or 119), that binds specifically to a Her2/Neu. It is believed to be operable to neutralize Her2/Neu, including, but not limited to a Her2/Neu family member from human, monkey, rat, or mouse.

According to other embodiments, the antibody or antigen-binding fragment of the present invention may be an antibody comprising variable light domain (V_{L}) comprising amino acid sequences according to SEQ ID NOs: 82, 91, 100, 109 or 118, respectively, that binds specifically to a Her2/Neu. It is believed to be operable to neutralize Her2/Neu, including, but not limited to a Her2/Neu family member from human, monkey, rat, or mouse.

In embodiments, the antibody or antigen binding fragment thereof of the present invention may further comprise four variable heavy domain framework regions (HFR)(HFR 1, 2, 3 and 4), which comprise an amino acid sequence comprising: HFR 1: VQLVQSGAEVKKPGESLKISCKGS (SEQ ID NOs: 15, 24, 33, 42 or 51), HFR 2: IAWVRQMPGKGLEYMGL (SEQ ID NOs: 16, 25, 34, 43 or 52), HFR 3: KYSPSFQGQVTISVDKSVSTAYLQWSSLKPSDSAVYFC (SEQ ID NOs: 17, 26, 35, 44 or 53), and HFR 4: WGQGTLVTV (SEQ ID NOs: 18, 27, 36, 45 or 54).

In other embodiments, the antibody or antigen binding fragment thereof of the present invention may further comprise four variable light domain framework regions (LFR)(LFR 1, 2, 3 and 4) which comprise an amino acid sequence comprising: LFR 1: QSVLTQPPSVSAAPGQKVTISCSGS (SEQ ID NOs: 84, 93, 102, 111, or 120), LFR 2: VSWYQQLPGTAPKLLIY (SEQ ID NOs: 85, 94, 103, 112, or 121), LFR 3: NRPAGVPDRFSGSKSGTSASLAISGFRSEDEADYYC (SEQ ID NOs: 86, 95, 104, 113 or 122), and LFR 4: FGGGTKVTVL (SEQ ID NOs: 87, 96, 105, 114, or 123).

According to another embodiment, the antibody or antigen binding fragment thereof of the present invention may comprise a variable heavy domain (V_{H}) comprising amino acid sequence comprising: VQLVQSGAEVKKPGESLKISCKGSGYSFTSYWIAWVRQMPGKGLEYMGLIYPGESDTKYSPSFQG QVTISVDKSVSTAYLQWSSLKPSDSAVYFCARHDVGYCTDRTCAKWPEYFQHWGQGTLVTV (SEQ ID NO: 23), and a variable light domain (V_{L}) comprising amino acid sequence comprising: QSVLTQPPSVSAAPGQKVTISCSGSSSNIGNNYVSWYQQLPGTAPKWYDHTNRPAGVPDRFSGS KSGTSASLAISGFRSEDEADYYCASWDYTLSGVWFGGGTKVTVL (SEQ ID NO: 83).

TABLE A
Amino acid sequence of Her2/Neu specific heavy variable heavy domain (V_{L}). Framework
regions (FRs) and complementarity-determining regions (CDRs) are classified and numbered
according to IMGT.

| | |
|---|---|
| | <------FR1--IMGT-------><-CDR1-><-- |
| VH_Antibody_0 | KLAATMEWSWVFLFFLSVTTGVHSQVQLVQSGAEVKKPGESLKISCKGSGYSFTSYWIAW |
| VH_Antibody_1 | KLAATMEWSWVFLFFLSVTTGVHSQVQLVQSGAEVKKPGESLKISCKGSGYSFTSYWIAW |
| VH_Antibody_2 | KLAATMEWSWVFLFFLSVTTGVHSQVQLVQSGAEVKKPGESLKISCKGSGYSFTSYWIAW |
| VH_Antibody_3 | KLAATMEWSWVFLFFLSVTTGVHSQVQLVQSGAEVKKPGESLKISCKGSGYSFTSYWIAW |
| VH_Antibody_4 | KLAATMEWSWVFLFFLSVTTGVHSQVQLVQSGAEVKKPGESLKISCKGSGYSFTSYWIAW |
| VH_Antibody_5 | KLAATMEWSWVFLFFLSVTTGVHSQVQLVQSGAEVKKPGESLKISCKGSGYSFTSYWIAW |
| | ************************************************************ |
| | FR2-IMGT-----><-CDR2-><-----------FR3-IMGT-----------------> |
| VH_Antibody_0 | VRQMPGKGLEYMGLIYPGDSDTKYSPSFQGQVTISVDKSVSTAYLQWSSLKPSDSAVYFC |
| VH_Antibody_1 | VRQMPGKGLEYMGLIYPGESDTKYSPSFQGQVTISVDKSVSTAYLQWSSLKPSDSAVYFC |
| VH_Antibody_2 | VRQMPGKGLEYMGLIYPGQSDTKYSPSFQGQVTISVDKSVSTAYLQWSSLKPSDSAVYFC |
| VH_Antibody_3 | VRQMPGKGLEYMGLIYPGSSDTKYSPSFQGQVTISVDKSVSTAYLQWSSLKPSDSAVYFC |
| VH_Antibody_4 | VRQMPGKGLEYMGLIYPGDTDTKYSPSFQGQVTISVDKSVSTAYLQWSSLKPSDSAVYFC |
| VH_Antibody_5 | VRQMPGKGLEYMGLIYPGDIDTKYSPSFQGQVTISVDKSVSTAYLQWSSLKPSDSAVYFC |
| | ************************************************************ |
| | <-----CDR3-IMGT------><--FR4--> |
| VH_Antibody_0 | ARHDVGYCTDRTCAKWPEYFQHWGQGTLVTVSSASTQSPSVFPLTRCCKNIPSNATSVTL |
| VH_Antibody_1 | ARHDVGYCTDRTCAKWPEYFQHWGQGTLVTVSSASTQSPSVFPLTRCCKNIPSNATSVTL |
| VH_Antibody_2 | ARHDVGYCTDRTCAKWPEYFQHWGQGTLVTVSSASTQSPSVFPLTRCCKNIPSNATSVTL |
| VH_Antibody_3 | ARHDVGYCTDRTCAKWPEYFQHWGQGTLVTVSSASTQSPSVFPLTRCCKNIPSNATSVTL |
| VH_Antibody_4 | ARHDVGYCTDRTCAKWPEYFQHWGQGTLVTVSSASTQSPSVFPLTRCCKNIPSNATSVTL |
| VH_Antibody_5 | ARHDVGYCTDRTCAKWPEYFQHWGQGTLVTVSSASTQSPSVFPLTRCCKNIPSNATSVTL |
| | ************************************************************ |
| VH_Antibody_0 | GCLATGYFPEPVMVTWDTGSLNGTTMTLPATTLTLSGHYATISLLTVSGAWAKQMFTCRV |
| VH_Antibody_1 | GCLATGYFPEPVMVTWDTGSLNGTTMTLPATTLTLSGHYATISLLTVSGAWAKQMFTCRV |
| VH_Antibody_2 | GCLATGYFPEPVMVTWDTGSLNGTTMTLPATTLTLSGHYATISLLTVSGAWAKQMFTCRV |
| VH_Antibody_3 | GCLATGYFPEPVMVTWDTGSLNGTTMTLPATTLTLSGHYATISLLTVSGAWAKQMFTCRV |
| VH_Antibody_4 | GCLATGYFPEPVMVTWDTGSLNGTTMTLPATTLTLSGHYATISLLTVSGAWAKQMFTCRV |
| VH_Antibody_5 | GCLATGYFPEPVMVTWDTGSLNGTTMTLPATTLTLSGHYATISLLTVSGAWAKQMFTCRV |
| | ************************************************************ |
| VH_Antibody_0 | AHTPSSTDWVDNKTFSVCSRDFTPPTVKILQSSCDGGGHFPPTIQLLCLVSGYTPGTINI |
| VH_Antibody_1 | AHTPSSTDWVDNKTFSVCSRDFTPPTVKILQSSCDGGGHFPPTIQLLCLVSGYTPGTINI |
| VH_Antibody_2 | AHTPSSTDWVDNKTFSVCSRDFTPPTVKILQSSCDGGGHFPPTIQLLCLVSGYTPGTINI |
| VH_Antibody_3 | AHTPSSTDWVDNKTFSVCSRDFTPPTVKILQSSCDGGGHFPPTIQLLCLVSGYTPGTINI |
| VH_Antibody_4 | AHTPSSTDWVDNKTFSVCSRDFTPPTVKILQSSCDGGGHFPPTIQLLCLVSGYTPGTINI |
| VH_Antibody_5 | AHTPSSTDWVDNKTFSVCSRDFTPPTVKILQSSCDGGGHFPPTIQLLCLVSGYTPGTINI |
| | ************************************************************ |
| VH_Antibody_0 | TWLEDGQVMDVDLSTASTTQEGELASTQSELTLSQKHWLSDRTYTCQVTYQGHTFEDSTK |
| VH_Antibody_1 | TWLEDGQVMDVDLSTASTTQEGELASTQSELTLSQKHWLSDRTYTCQVTYQGHTFEDSTK |
| VH_Antibody_2 | TWLEDGQVMDVDLSTASTTQEGELASTQSELTLSQKHWLSDRTYTCQVTYQGHTFEDSTK |
| VH_Antibody_3 | TWLEDGQVMDVDLSTASTTQEGELASTQSELTLSQKHWLSDRTYTCQVTYQGHTFEDSTK |
| VH_Antibody_4 | TWLEDGQVMDVDLSTASTTQEGELASTQSELTLSQKHWLSDRTYTCQVTYQGHTFEDSTK |
| VH_Antibody_5 | TWLEDGQVMDVDLSTASTTQEGELASTQSELTLSQKHWLSDRTYTCQVTYQGHTFEDSTK |
| | ************************************************************ |
| VH_Antibody_0 | KCADSNPRGVSAYLSRPSPFDLFIRKSPTITCLVVDLAPSKGTVNLTWSRASGKPVNHST |
| VH_Antibody_1 | KCADSNPRGVSAYLSRPSPFDLFIRKSPTITCLVVDLAPSKGTVNLTWSRASGKPVNHST |
| VH_Antibody_2 | KCADSNPRGVSAYLSRPSPFDLFIRKSPTITCLVVDLAPSKGTVNLTWSRASGKPVNHST |
| VH_Antibody_3 | KCADSNPRGVSAYLSRPSPFDLFIRKSPTITCLVVDLAPSKGTVNLTWSRASGKPVNHST |
| VH_Antibody_4 | KCADSNPRGVSAYLSRPSPFDLFIRKSPTITCLVVDLAPSKGTVNLTWSRASGKPVNHST |
| VH_Antibody_5 | KCADSNPRGVSAYLSRPSPFDLFIRKSPTITCLVVDLAPSKGTVNLTWSRASGKPVNHST |
| | ************************************************************ |
| VH_Antibody_0 | RKEEKQRNGTLTVTSTLPVGTRDWIEGETYQCRVTHPHLPRALMRSTTKTSGPRAAPEVY |
| VH_Antibody_1 | RKEEKQRNGTLTVTSTLPVGTRDWIEGETYQCRVTHPHLPRALMRSTTKTSGPRAAPEVY |
| VH_Antibody_2 | RKEEKQRNGTLTVTSTLPVGTRDWIEGETYQCRVTHPHLPRALMRSTTKTSGPRAAPEVY |
| VH_Antibody_3 | RKEEKQRNGTLTVTSTLPVGTRDWIEGETYQCRVTHPHLPRALMRSTTKTSGPRAAPEVY |
| VH_Antibody_4 | RKEEKQRNGTLTVTSTLPVGTRDWIEGETYQCRVTHPHLPRALMRSTTKTSGPRAAPEVY |
| VH_Antibody_5 | RKEEKQRNGTLTVTSTLPVGTRDWIEGETYQCRVTHPHLPRALMRSTTKTSGPRAAPEVY |
| | ************************************************************ |
| VH_Antibody_0 | AFATPEWPGSRDKRTLACLIQNFMPEDISVQWLHNEVQLPDARHSTTQPRKTKGSGFFVF |
| VH_Antibody_1 | AFATPEWPGSRDKRTLACLIQNFMPEDISVQWLHNEVQLPDARHSTTQPRKTKGSGFFVF |
| VH_Antibody_2 | AFATPEWPGSRDKRTLACLIQNFMPEDISVQWLHNEVQLPDARHSTTQPRKTKGSGFFVF |
| VH_Antibody_3 | AFATPEWPGSRDKRTLACLIQNFMPEDISVQWLHNEVQLPDARHSTTQPRKTKGSGFFVF |
| VH_Antibody_4 | AFATPEWPGSRDKRTLACLIQNFMPEDISVQWLHNEVQLPDARHSTTQPRKTKGSGFFVF |
| VH_Antibody_5 | AFATPEWPGSRDKRTLACLIQNFMPEDISVQWLHNEVQLPDARHSTTQPRKTKGSGFFVF |
| | ************************************************************ |
| VH_Antibody_0 | SRLEVTRAEWEQKDEFICRAVHEAASPSQTVQRAVSVNPGK |
| VH_Antibody_1 | SRLEVTRAEWEQKDEFICRAVHEAASPSQTVQRAVSVNPGK |
| VH_Antibody_2 | SRLEVTRAEWEQKDEFICRAVHEAASPSQTVQRAVSVNPGK |
| VH_Antibody_3 | SRLEVTRAEWEQKDEFICRAVHEAASPSQTVQRAVSVNPGK |
| VH_Antibody_4 | SRLEVTRAEWEQKDEFICRAVHEAASPSQTVQRAVSVNPGK |
| VH_Antibody_5 | SRLEVTRAEWEQKDEFICRAVHEAASPSQTVQRAVSVNPGK |
| | ***************************************** |

Antibody 0 represent the starting antibody and 1 to 5 the mutated sequences. Underlined = amino acid sequence of variable region from FR1 to FR4.

TABLE B
Amino acid sequence of Her2/Neu specific light variable heavy domain (V_{L}). Framework
regions (FRs) and complementarity-determining regions (CDRs) are classified and numbered
according to IMGT.

| | | |
|---|---|---|
| | <-------FR1-IMGT--------><-CDR1-><- | |
| VL_antibody_0 | KLAATMSVPTQVLGLLLLWLTDARCQSVLTQPPSVSAAPGQKVTISCSGSSSNIGNNYVS | 60 |
| VL_antibody_1 | KLAATMSVPTQVLGLLLLWLTDARCQSVLTQPPSVSAAPGQKVTISCSGSSSNIGNNYVS | 60 |
| VL_antibody_2 | KLAATMSVPTQVLGLLLLWLTDARCQSVLTQPPSVSAAPGQKVTISCSGSSSNIGNNYVS | 60 |
| VL_antibody_3 | KLAATMSVPTQVLGLLLLWLTDARCQSVLTQPPSVSAAPGQKVTISCSGSSSNIGNNYVS | 60 |
| VL_antibody_4 | KLAATMSVPTQVLGLLLLWLTDARCQSVLTQPPSVSAAPGQKVTISCSGSSSNIGNNYVS | 60 |
| VL_antibody_5 | KLAATMSVPTQVLGLLLLWLTDARCQSVLTQPPSVSAAPGQKVTISCSGSSSNIGNNYVS | 60 |
| | ************************************************************ | |
| | --FR2-IMGT----><CDR2><------------FR3-IMGT--------------><-CDR3 | |
| VL_antibody_0 | WYQQLPGTAPKLLIY-DHT--NRPAGVPDRFSGSKSGTSASLAISGFRSEDEADYYCASWDYT | 120 |
| VL_antibody_1 | WYQQLPGTAPKLLIY-DHT--NRPAGVPDRFSGSKSGTSASLAISGFRSEDEADYYCASWDYT | 120 |
| VL_antibody_2 | WYQQLPGTAPKLLIY-DHT--NRPAGVPDRFSGSKSGTSASLAISGFRSEDEADYYCASWDYT | 120 |
| VL_antibody_3 | WYQQLPGTAPKLLIY-DHT--NRPAGVPDRFSGSKSGTSASLAISGFRSEDEADYYCASWDYT | 120 |
| VL_antibody_4 | WYQQLPGTAPKLLIY-DHT--NRPAGVPDRFSGSKSGTSASLAISGFRSEDEADYYCASWDYT | 120 |
| VL_antibody_5 | WYQQLPGTAPKLLIY-DHT--NRPAGVPDRFSGSKSGTSASLAISGFRSEDEADYYCASWDYT | 120 |
| | ************************************************************ | |
| | ----><--FR4---> | |
| VL_antibody_0 | LSGWVFGGGTKVTVLGQPKAAPSVTLFPPSSEELQANKATLVCLISDFYPGAVTVAWKAD | 180 |
| VL_antibody_1 | LSGWVFGGGTKVTVLGQPKAAPSVTLFPPSSEELQANKATLVCLISDFYPGAVTVAWKAD | 180 |
| VL_antibody_2 | LSGWVFGGGTKVTVLGQPKAAPSVTLFPPSSEELQANKATLVCLISDFYPGAVTVAWKAD | 180 |
| VL_antibody_3 | LSGWVFGGGTKVTVLGQPKAAPSVTLFPPSSEELQANKATLVCLISDFYPGAVTVAWKAD | 180 |
| VL_antibody_4 | LSGWVFGGGTKVTVLGQPKAAPSVTLFPPSSEELQANKATLVCLISDFYPGAVTVAWKAD | 180 |
| VL_antibody_5 | LSGWVFGGGTKVTVLGQPKAAPSVTLFPPSSEELQANKATLVCLISDFYPGAVTVAWKAD | 180 |
| | ************************************************************ | |
| VL_antibody_0 | SSPVKAGVETTTPSKQSNNKYAASSYLSLTPEQWKSHRSYSCQVTHEGSTVEKTVAPTEC | 240 |
| VL_antibody_1 | SSPVKAGVETTTPSKQSNNKYAASSYLSLTPEQWKSHRSYSCQVTHEGSTVEKTVAPTEC | 240 |
| VL_antibody_2 | SSPVKAGVETTTPSKQSNNKYAASSYLSLTPEQWKSHRSYSCQVTHEGSTVEKTVAPTEC | 240 |
| VL_antibody_3 | SSPVKAGVETTTPSKQSNNKYAASSYLSLTPEQWKSHRSYSCQVTHEGSTVEKTVAPTEC | 240 |
| VL_antibody_4 | SSPVKAGVETTTPSKQSNNKYAASSYLSLTPEQWKSHRSYSCQVTHEGSTVEKTVAPTEC | 240 |
| VL_antibody_5 | SSPVKAGVETTTPSKQSNNKYAASSYLSLTPEQWKSHRSYSCQVTHEGSTVEKTVAPTEC | 240 |
| | ************************************************************ | |
| VL_antibody_0 | S | 241 |
| VL_antibody_1 | S | 241 |
| VL_antibody_2 | S | 241 |
| VL_antibody_3 | S | 241 |
| VL_antibody_4 | S | 241 |
| VL_antibody_5 | S | 241 |
| | * | |

Antibody 0 represent the starting antibody and 1 to 5 the mutated sequences. Underlined = amino acid sequence of variable region from FR1 to FR4. The dash (-) character is onlyillustrative of a space in the text to identify the CDR2, and not of a CDR2 feature.

According to other embodiments, the antibody or an antigen-binding fragment of the present invention may have sequences substantially identical to the sequences disclosed above, operable to bind to Her2/Neu. A substantially identical sequence may comprise one or more conservative amino acid mutations. It is known in the art that the introduction of one or more conservative amino acid mutations to a reference sequence may yield a mutant peptide with no substantial change in physiological, chemical, physico-chemical or functional properties compared to the reference sequence. In such a case, the reference and mutant sequences would be considered “substantially identical” polypeptides. A conservative amino acid substitution is defined herein as the substitution of an amino acid residue for another amino acid residue with similar chemical properties (e.g. size, charge, or polarity). According to one embodiment, one or more conservative amino acid mutations may be made to one or more framework regions of the sdAb while maintaining both the CDR sequences listed above and the overall structure of the CDR of the antibody or antigen-binding fragment; thus the specificity and binding of the antibody are maintained. According to another embodiment, one or more conservative amino acid mutations may be made to one or more framework regions of the sdAb and to a CDR sequence listed above while maintaining the antigen-binding function of the overall structure of the CDR of the antibody or antigen-binding fragment; thus the specificity and binding of the antibody are maintained.

In a non-limiting example, a conservative mutation may be a conservative amino acid substitution. Such a conservative amino acid substitution may substitute a basic, neutral, hydrophobic, or acidic amino acid for another amino acid of the same group. By the term “basic amino acid” it is meant a hydrophilic amino acid having a side chain pK value of greater than 7, which is typically positively charged at physiological pH. Basic amino acids include histidine (His or H), arginine (Arg or R), and lysine (Lys or K). By the term “neutral amino acid” (also “polar amino acid”), it is meant a hydrophilic amino acid having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). By the term “hydrophobic amino acid” (also “non-polar amino acid”) it is meant an amino acid exhibiting a hydrophobicity of greater than zero according to the normalized consensus hydrophobicity scale of Eisenberg (1984). Hydrophobic amino acids include proline (Pro or P), isoleucine (Ile or I), phenylalanine (Phe or F), valine (Val or V), leucine (Leu or L), tryptophan (Trp or \AO, methionine (Met or M), alanine (Ala or A), and glycine (Gly or G). “Acidic amino acid” refers to a hydrophilic amino acid having a side chain pK value of less than 7, which is typically negatively charged at physiological pH. Acidic amino acids include glutamate (Glu or E) and aspartate (Asp or D).

Sequence identity is used to evaluate the similarity of two sequences. It is determined by calculating the percentage of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 90% identical; in another example, the substantially identical sequences may be at least 90%, at least 91%, at least 92%, at least 93%, at least 94%, at least 95%, at least 96%, at least 97%, at least 98%, or at least 99% identical, or any percentage therebetween, at the amino acid level to sequences described herein. Importantly, a substantially identical sequence retains the activity and specificity of the reference sequence. In a non-limiting embodiment, the difference in sequence identity may be due to one or more conservative amino acid mutations. In a non-limiting example, the present invention may be directed to an antibody or antigen-binding fragment comprising a sequence at least 95%, at least 98%, or at least 99% identical to that of one or more of the antibodies or antigen-binding fragments described herein.

The antibody or an antigen-binding fragment of the present invention may be used, for example, to bind specifically to a Her2/Neu. It is believed to neutralize the Her2/Neu receptors on the surface of cancer cells and blocking them from receiving growth signals. As used herein, the expression “bind specifically to a Her2/Neu” or “bind specifically to Her2/Neu” is intended to mean that the antibody or antigen-binding fragment of the present invention is enabled to bind specifically to Her2/Neu and particularly to one or more human, rhesus, mouse or rat serum Her2/Neu.

The term “antibody”, also referred to in the art as “immunoglobulin” (Ig), as used herein refers to a protein constructed from paired heavy and light polypeptide chains; various Ig isotypes exist, including IgA, IgD, IgE, IgG, and IgM. When an antibody is correctly folded, each chain folds into a number of distinct globular domains joined by more linear polypeptide sequences. For example, the immunoglobulin light chain folds into a variable (V_{L}) and a constant (CO domain, while the heavy chain folds into a variable (V_{H}) and three constant (C_{H}1, C_{H}2, C_{H}3) domains. Interaction of the heavy and light chain variable domains (V_{H} and V_{L}) results in the formation of an antigen binding region (Fv). Each domain has a well-established structure familiar to those of skill in the art.

The light and heavy chain variable regions are responsible for binding the target antigen and can therefore show significant sequence diversity between antibodies. The constant regions show less sequence diversity, and are responsible for binding a number of natural proteins to elicit important biochemical events. The variable region of an antibody contains the antigen-binding determinants of the molecule, and thus determines the specificity of an antibody for its target antigen. The majority of sequence variability occurs in six hypervariable regions, three each per variable heavy (V_{H}) and light (V_{L}) chain; the hypervariable regions combine to form the antigen-binding site, and contribute to binding and recognition of an antigenic determinant. The specificity and affinity of an antibody for its antigen is determined by the structure of the hypervariable regions, as well as their size, shape, and chemistry of the surface they present to the antigen. Various schemes exist for identification of the regions of hypervariability, the two most common being those of Kabat and of Chothia and Lesk. Kabat and Wu (1991) define the “complementarity-determining regions” (CDRs) based on sequence variability at the antigen-binding regions of the V_{H} and V_{L} domains. Chothia and Lesk (1987) define the “hypervariable loops” (H or L) based on the location of the structural loop regions in the V_{H} and V_{L} domains. These individual schemes define CDR and hypervariable loop regions that are adjacent or overlapping. Those of skill in the antibody art often utilize the terms “CDR” and “hypervariable loop” interchangeably, and they may be so used herein. The CDRs/loops are identified herein according to the IMGT nomenclature scheme (i.e., CDR1, 2 and 3, for each variable region).

An “antibody fragment” or “antigen-binding fragment” as referred to herein may include any suitable antigen-binding antibody fragment known in the art. The antibody fragment may be a naturally-occurring antibody fragment, or it may be a non-naturally occurring antibody fragment obtained, for example, by manipulation of a naturally-occurring antibody or by recombinant methods. For example, an antibody fragment may include, but is not limited to, a Fv, a single-chain Fv (scFv; a molecule consisting of V_{L} and V_{H} connected with a peptide linker), a Fab, a F(ab′)2, or a single-domain antibody (sdAb; a fragment composed of a single V_{L} or V_{H} or a V_{H}H), or a multivalent presentation of any of these. Antibody fragments such as those just described may require one or more linker sequences, disulfide bonds, or other types of covalent bond to link different portions of the fragments. Those of skill in the art will be familiar with the requirements of the different types of fragments and various approaches for their construction.

In a non-limiting example, the antigen-binding fragment of the present invention may be a sdAb derived from a naturally-occurring source. Heavy chain antibodies of camelid origin (Hamers-Casterman et al, 1993) lack light chains and thus their antigen binding sites consist of one domain, termed V_{H}H. SdAbs have also been observed in shark and are termed V_{NAR} (Nuttall et al, 2003). Other sdAbs may be engineered based on human Ig heavy and light chain sequences (Jespers et al, 2004; To et al, 2005). As used herein, the term “sdAb” includes an sdAb directly isolated from a V_{H}, V_{H}H, V_{L}, or V_{NAR} reservoir of any origin through phage display or other technology, an sdAb derived from the aforementioned sdAb, a recombinantly produced sdAb, as well as an sdAb generated through further modification of such sdAb by humanization, affinity maturation, stabilization, solubilization, camelization, or other methods of antibody engineering. Also encompassed by the present invention are homologues, derivatives, or fragments that retain the antigen-binding function and specificity of the sdAb.

SdAbs possess desirable properties for antibody molecules, such as high thermostability, high detergent resistance, relatively high resistance to proteases (Dumoulin et al, 2002) and high production yield (Arbabi-Ghahroudi et al, 1997). They can also be engineered to have very high affinity by isolation from an immune library (Li et al, 2009) or by in vitro affinity maturation (Davies & Riechmann, 1996). Further modifications to increase stability, such as the introduction of one or more non-canonical disulfide bonds (Hussack et al, 2011a, b; Kim et al, 2012), may also be brought to the sdAb.

A person of skill in the art would be well-acquainted with the structure of a single-domain antibody (see, for example, 3DWT, 2P42 in Protein Data Bank). An sdAb comprises a single immunoglobulin domain that retains the immunoglobulin fold; most notably, only three CDR/hypervariable loops form the antigen-binding site. However, and as would be understood by those of skill in the art, not all CDRs may be required for binding the antigen. For example, and without wishing to be limiting, one, two, or three of the CDRs may contribute to binding and recognition of the antigen by the sdAb of the present invention. The CDRs of the sdAb or variable domain are referred to herein as CDR1, CDR2, and CDR3.

The present invention further encompasses an antibody or an antigen-binding fragment that is “humanized” using any suitable method known in the art, such as, but not limited to, CDR grafting or veneering. Humanization of an antibody or an antigen-binding fragment comprises replacing an amino acid in the antibody or antigen-binding fragment sequence with its human counterpart, as found in the human consensus sequence, without substantial loss of antigen-binding ability or specificity; this approach reduces immunogenicity of the antibody or antigen-binding fragment when introduced into human subjects. In the process of CDR grafting, one or more than one of the CDRs defined herein may be fused or grafted to a human variable region (V_{H}, or V_{L}), to a human antibody (IgA, IgD, IgE, IgG, and IgM), to a human antibody fragment framework region (Fv, scFv, Fab) or to another protein of similar size and nature onto which a CDR can be grafted (Nicaise et al, 2004). In such a case, the conformation of the one or more than one hypervariable loop is likely preserved, and the affinity and specificity of the antibody or antigen-binding fragment for its target (i.e., a human/rhesus/rat/mouse Her2/Neu family member, collectively referred to as Her2/Neu family member) is likely minimally affected. CDR grafting is known in the art and is described in at least the following: U.S. Pat. Nos. 6,180,370, 5,693,761, 6,054,297, 5,859,205, and European Patent No. 626390. Veneering, also referred to in the art as “variable region resurfacing”, involves humanizing solvent-exposed positions of an antibody or antigen-binding fragment; thus, preserving buried non-humanized residues, which may be important for CDR conformation, while minimizing the potential for immunological reaction against solvent-exposed regions. Veneering is known in the art and is described in at least the following: U.S. Pat. Nos. 5,869,619, 5,766,886, 5,821,123, and European Patent No. 519596. Persons of skill in the art would also be amply familiar with methods of preparing such humanized antibody fragments and humanizing amino acid positions.

The antibody or antigen-binding fragment according to the present invention may comprise an additional sequence to aid in expression, detection or purification of the antibody or antigen-binding fragment. Any such sequence or tag known to those of skill in the art may be used. For example, and without wishing to be limiting, the antibody or antigen-binding fragment may comprise a targeting or signal sequence (such as, but not limited to, ompA or pelB), a detection/purification tag (such as, but not limited to, c-Myc, HA, His5, or His6), or a combination of any two or more thereof. In another example, the additional sequence may be a biotin recognition site, such as that described by Cronan et al. in WO 95/04069 or by Voges et al. in WO/2004/076670. As is also known to those of skill in the art, a linker sequence may be used in conjunction with the additional sequence or tag, or may serve as a detection/purification tag.

In another embodiment, there is disclosed a compound comprising an antibody or an antigen-binding fragment according to the present invention, linked to a functional moiety, optionally by a linker sequence. In embodiments of the compound, the antibody or antigen-binding fragment may be linked to the functional moiety via a linker (also known as a linker sequence). As used herein, the term “linker sequence” is intended to mean a short (typically 40 amino acids or fewer) peptide sequence that is introduced between protein domains. Linker sequences are often composed of flexible residues such as glycine and serine so that the linked protein domains are free to move relative to one another. The linker sequence can be any linker sequence known in the art that would allow for the antibody and the functional moiety of the present invention to be operably linked for the desired function. The linker may be any sequence known in the art (either a natural or synthetic linker) that allows for an operable fusion comprising an antibody or antigen-binding fragment linked to a polypeptide (e.g. the functional moiety). For example, the linker sequence may be a linker sequence L such as (GGGS)ₙ and (GGGGS)ₙ, wherein n is equal to or greater than 1, or from about 1 to about 5, or from about 1 to 15; or n may be any number that would allow for the operability of the compound of the present invention. In another example, the linker may be an amino acid sequence, for example, an amino acid sequence that comprises about 1 to about 40 amino acids, or about 3 to about 40 amino acids, or about 5 to about 40 amino acids, or about 10 to about 40 amino acids, or about 15 to about 40 amino acids, or about 20 to about 40 amino acids, or about 25 to about 40 amino acids, or about 30 to about 40 amino acids, or about 35 to about 40 amino acids, or about 3 to about 35 amino acids, or about 5 to about 35 amino acids, or about 10 to about 35 amino acids, or about 15 to about 35 amino acids, or about 20 to about 35 amino acids, or about 25 to about 35 amino acids, or about 30 to about 35 amino acids, or about 3 to about 30 amino acids, or about 5 to about 30 amino acids, or about 10 to about 30 amino acids, or about 15 to about 30 amino acids, or about 20 to about 30 amino acids, or about 25 to about 30 amino acids, or about 3 to about 25 amino acids, or about 5 to about 25 amino acids, or about 10 to about 25 amino acids, or about 15 to about 25 amino acids, or about 20 to about 25 amino acids, or about 3 to about 20 amino acids, or about 5 to about 20 amino acids, or about 10 to about 20 amino acids, or about 15 to about 20 amino acids, or about 3 to about 15 amino acids, or about 5 to about 15 amino acids, or about 10 to about 15 amino acids, or about 15 to about 20 amino acids, or about 3 to about 10 amino acids, or about 5 to about 10 amino acids, or about 3 to about 5 amino acids, or up to 3, up to 5, up to 10, up to 15, up to 20, up to 25, up to 30, up to 35, or up to 40 amino acids.

As used herein, the term “functional moiety” is intended to mean a part of the compound having an activity, purpose, or task; relating to the way in which the compound is intended to work or operate. In embodiments, the functional moiety may be linked to the antibody or antigen-binding fragment, for example, through a chemical link pursuant to a chemical reaction, and/or through fusion of the antibody or antigen-binding fragment with the functional moiety, obtained for example using recombinant DNA technology. According to an embodiment, the antibody or antigen-binding fragment of the compound may be fused to a peptide, a polypeptide (e.g. growth factor CIBP2, an antimicrobial cyclic peptide), a protein, an enzyme [such as iduronate-2-sulfatase (IDS), acid beta-glucosidase (GCase), a serine protease, a growth factor, etc.], another (or the same) antibody or a fragment operable to bind a target epitope (e.g. an anti-microbial antibody, an anti-inflammatory antibody, an intrabody, a BBB-crossing antibody, a neurodegeneration target antibody, an ion channel targeting antibody, a cancer associated antigen antibody, a checkpoint inhibitor targeting antibody, or a GPCR targeting antibody)(for any use and for example for use in imaging, diagnostic, affinity purification, etc.), or a combination of any two or more thereof, in which both the antibody or antigen-binding fragment and the rest of the compound (i.e. the functional moiety) remain functional for their intended purpose. In a preferred embodiment, the compound may be fused to a second antibody or antigen-binding fragment, operable to bind a target epitope, which may be the same as, or distinct from the epitope of the antibody or antigen-binding fragment of the present invention.

The antibody or antigen-binding fragment of the present invention may also be in a multivalent display format, also referred to herein as multivalent presentation. Multimerization may be achieved by any suitable method known in the art. For example, and without wishing to be limiting in any manner, multimerization may be achieved using self-assembly molecules such as those described in Zhang et al (2004a; 2004b) and WO2003/046560, where pentabodies are produced by expressing a fusion protein comprising the antibody or antigen-binding fragment of the present invention and the pentamerization domain of the B-subunit of an AB5 toxin family (Merritt & Hol, 1995). A multimer may also be formed using the multimerization domains described by Zhu et al. (2010); this form, referred to herein as a “combody” form, is a fusion of the antibody or fragment of the present invention with a coiled-coil peptide resulting in a multimeric molecule (Zhu et al., 2010). Other forms of multivalent display are also encompassed by the present invention. For example, and without wishing to be limiting, the antibody or antigen-binding fragment may be presented as a dimer, a trimer, or any other suitable oligomer. This may be achieved by methods known in the art (Spiess et al, 2015), for example by direct linking connection (Nielsen et al, 2000), c-jun/Fos interaction (de Kruif & Logtenberg, 1996), or “Knob into holes” interaction (Ridgway et al, 1996).

Another method known in the art for multimerization is to dimerize the antibody or antigen-binding fragment using an Fc domain, such as, but not limited to a human Fc domain. The Fc domain may be selected from various classes including, but not limited to, IgG, IgM, or various subclasses including, but not limited to IgG1, IgG2, etc. In this approach, the Fc gene is inserted into a vector along with the sdAb gene to generate a sdAb-Fc fusion protein (Bell et al, 2010; Iqbal et al, 2010); the fusion protein is recombinantly expressed, then purified. For example, and without wishing to be limiting in any manner, a multivalent display format may encompass a chimeric or humanized format of V_{H}H of the present invention linked to an Fc domain, or bi or tri-specific antibody fusions with two or three V_{H}Hs recognizing unique epitopes. Such antibodies are easy to engineer and produce, can greatly extend the serum half-life of a sdAb, and may be excellent tumor imaging reagents (Bell et al., 2010).

The Fc domain in the multimeric complex as just described may be any suitable Fc fragment known in the art. The Fc fragment may be from any suitable source; for example, the Fc fragment may be of mouse or human origin. In a specific, non-limiting example, the Fc fragment may be a mouse Fc2b fragment or a human Fc1 fragment (Bell et al, 2010; Iqbal et al, 2010). The Fc fragment may be fused to the N-terminal or C-terminal end of the V_{H}H or humanized version of the present invention.

Each subunit of the multimers described above may comprise the same or different antibodies or antigen-binding fragments of the present invention, which may have the same or different specificity. Additionally, the multimerization domains may be linked to the antibody or antigen-binding fragment using a linker, as required; such a linker should be of sufficient length and appropriate composition to provide flexible attachment of the two molecules but should not hamper the antigen-binding properties of the antibody or antigen-binding fragment. As defined above, the linker sequence can be any linker known in the art that would allow for the compound of the present invention to be prepared and be operable for the desired function.

According to another embodiment, the present invention also encompasses a composition comprising one or more than one compound as described herein. The composition may comprise a single antibody and/or compound as described above, or the composition may comprise a mixture of antibody and/or compounds. Furthermore, in a composition comprising a mixture of antibody and/or compounds of the present invention, the antibody and/or compounds may have the same specificity, or they may differ in their specificities; for example, and without wishing to be limiting in any manner, the composition may comprise antibody and/or compounds specific to Her2/Neu (same or different epitope).

A composition according to the invention may also comprise a pharmaceutically acceptable diluent, excipient, or carrier. The diluent, excipient, or carrier may be any suitable diluent, excipient, or carrier known in the art that is compatible with other ingredients in the composition, that is compatible with the method of delivery of the composition, and that is not deleterious to the recipient of the composition. The composition may be in any suitable form; for example, the composition may be provided in suspension form, powder form (such as, but not limited to, lyophilised or encapsulated), capsule form or tablet form. For example, and without wishing to be limiting, when the composition is provided in suspension form, the carrier may comprise water, saline, or a suitable buffer, and optionally comprise one or more additives to improve solubility and/or stability. Reconstitution to produce a suspension may be effected in a buffer at a suitable pH to ensure the viability of the antibody or antigen-binding fragment. Dry powders may also include additives to improve stability and/or carriers to increase bulk/volume; for example, and without wishing to be limiting, the dry powder composition may comprise sucrose or trehalose. In a specific, non-limiting example, the composition may be formulated for delivery of the antibody or antigen-binding fragment to the gastrointestinal tract of the subject. Thus, the composition may comprise encapsulation, time release, or other suitable technologies for delivery of the antibody and/or compound of the present invention. It would be within the competency of a person of skill in the art to prepare suitable compositions comprising the present antibody and/or compound.

The invention also encompasses a nucleic acid molecule comprising a nucleotide sequence encoding an antibody, antigen-binding fragment, or compound of the present invention. The invention further comprises a vector comprising the nucleic acid molecule; a cell comprising the vector, for expressing the antibody, antigen-binding fragment, or compound of the present invention, and a cell for expressing the antibody, antigen-binding fragment, or compound of the present invention.

According to another embodiment, there is provided a method of treating a Her2/Neu associated disease comprising administering the antibody or antigen-binding fragment of the present invention, the compound of the present invention, the composition of the present invention or a combination of any two or more thereof to a subject in need thereof.

According to another embodiment, there is provided a use of the antibody or antigen-binding fragment of the present invention, the compound of the present invention, the composition of the present invention or a combination of any two or more thereof for the treatment of a Her2/Neu associated disease.

According to another embodiment, there is provided a use of the antibody or antigen-binding fragment of the present invention, the compound of the present invention, the composition of the present invention or a combination of any two or more thereof in the manufacture of a medicament for the treatment of a Her2/Neu associated disease.

According to another embodiment, there is provided an antibody or antigen-binding fragment of the present invention, the compound of the present invention, the composition of the present invention or a combination of any two or more thereof for use in the treatment of a Her2/Neu associated disease.

As used herein, the expression “Her2/Neu associated disease” is intended to mean that the disease involves, is connected to, Her2/Neu as an etiological factor.

According to an embodiment, the disease is a cancer. Non-limiting examples in which Her2/Neu may be involved include ovarian cancer, breast cancer, stomach cancer, lung cancer, uterine cancer, salivary gland cancer, testicular germ cell cancer, bladder cancer, pancreatic cancer, and esophageal cancer.

According to another embodiment, the method of the present invention may further comprise administering a second therapeutic agent comprising at least one of a cytotoxic agent, an additional antibody or a therapeutically active fragment thereof, or a chemotherapy regimen.

Indeed, it is contemplated that the present method and therapeutic strategies may be used alone or in combination with cytotoxic agents to increase overall patient survival. The cytotoxic therapeutic agents include, but are not limited to, angiogenesis inhibitors, antiproliferative agents, kinase inhibitors, receptor tyrosine kinase inhibitors, aurora kinase inhibitors, polo-like kinase inhibitors, bcr-abl kinase inhibitors, growth factor inhibitors, COX-2 inhibitors, non-steroidal anti-inflammatory drugs (NSAIDS), antimitotic agents, alkylating agents, antimetabolites, intercalating antibiotics, platinum containing agents, growth factor inhibitors, ionizing radiation, cell cycle inhibitors, enzymes, topoisomerase inhibitors, biologic response modifiers, immunologicals, antibodies, hormonal therapies, retinoids/deltoids plant alkaloids, proteasome inhibitors, HSP-90 inhibitors, histone deacetylase inhibitors (HDAC) inhibitors, purine analogs, pyrimidine analogs, MEK inhibitors, CDK inhibitors, ErbB (such as ErbB2) receptor inhibitors, phosphoinositide 3-kinases (PI3Ks)/Akt signaling inhibitors, mTOR inhibitors and combinations thereof as well as other antitumor agents.

Angiogenesis inhibitors include, but are not limited to, EGFR inhibitors, PDGFR inhibitors, VEGFR inhibitors, TTE2 inhibitors, IGFIR inhibitors, matrix metalloproteinase 2 (MMP-2) inhibitors, matrix metalloproteinase 9 (MMP-9) inhibitors, thrombospondin analogs such as thrombospondin-1 and N—Ac-Sar-Gly-Val-D-allolle-Thr-Nva-He-Arg-Pro-NHCH₂CH₃ or a salt thereof and analogues of N—Ac-Sar-Gly-Val-D-allolle-Thr-Nva-lle-Arg-PrO—NHCH₂CH₃ such as N—Ac-GlyVal-D-alle-Ser-Gln-lle-Arg-ProNHCH₂CH₃ or a salt thereof.

Examples of EGFR inhibitors include, but are not limited to, Iressa (gefitinib), Tarceva (erlotinib or OSI-774), Icotinib, Erbitux (cetuximab), EMD-7200, ABX-EGF, HR3, IgA antibodies, TP-38 (IVAX), EGFR fusion protein, EGF-vaccine, anti-EGFr immunoliposomes, Tykerb (lapatinib) and AZD-8931 (sapitinib).

Examples of PDGFR inhibitors include, but are not limited to, CP-673,451 and CP-868596.

Examples of VEGFR inhibitors include, but are not limited to, Avastin (bevacizumab), Sutent (sunitinib, SUI 1248), Nexavar (sorafenib, BAY43-9006), CP-547,632, axitinib (AG13736), Apatinib, cabozantinib, Zactima (vandetanib, ZD-6474), AEE788, AZD-2171, VEGF trap, Vatalanib (PTK-787, ZK-222584), Macugen, M862, Pazopanib (GW786034), ABT-869 and angiozyme.

Examples of thrombospondin analogs include, but are not limited to, TSP-I and ABT-510.

Examples of aurora kinase inhibitors include, but are not limited to, VX-680, AZD-1152 and MLN-8054. Example of polo-like kinase inhibitors include, but are not limited to, BI-2536.

Examples of bcr-abl kinase inhibitors include, but are not limited to, Gleevec (imatinib) and Dasatinib (BMS354825).

Examples of platinum containing agents includes, but are not limited to, cisplatin, Paraplatin (carboplatin), eptaplatin, lobaplatin, nedaplatin, Eloxatin (oxaliplatin) or satraplatin.

Examples of mTOR inhibitors includes, but are not limited to, CCI-779, rapamycin, temsirolimus, everolimus, RAD001, INK-128 and ridaforolimus.

Examples of HSP-90 inhibitors includes, but are not limited to, geldanamycin, radicicol, 17-AAG, KOS-953, 17-DMAG, CNF-101, CNF-1010, 17-AAG-nab, NCS-683664, Mycograb, CNF-2024, PU3, PU24FC1, VER49009, IPI-504, SNX-2112 and STA-9090.

Examples of histone deacetylase inhibitors (HDAC) includes, but are not limited to, Suberoylanilide hydroxamic acid (SAHA), MS-275, valproic acid, TSA, LAQ-824, Trapoxin, tubacin, tubastatin, ACY-1215 and Depsipeptide.

Examples of MEK inhibitors include, but are not limited to, PD325901, ARRY-142886, ARRY-438162 and PD98059.

Examples of CDK inhibitors include, but are not limited to, flavopyridol, MCS-5A, CVT-2584, seliciclib (CYC-202, R-roscovitine), ZK-304709, PHA-690509, BMI-1040, GPC-286199, BMS-387,032, PD0332991 and AZD-5438.

Examples of COX-2 inhibitors include, but are not limited to, CELEBREX™ (celecoxib), parecoxib, deracoxib, ABT-963, MK-663 (etoricoxib), COX-189 Lumiracoxib), BMS347070, RS 57067, NS-398, Bextra (valdecoxib), paracoxib, Vioxx (rofecoxib), SD-8381, 4-Methyl-2-(3,4-dimethylphenyl)-l-(4-sulfamoyl-phenyl-IH-pyrrole, T-614, JTE-522, S-2474, SVT-2016, CT-3, SC-58125 and Arcoxia (etoricoxib).

Examples of non-steroidal anti-inflammatory drugs (NSAIDs) include, but are not limited to, Salsalate (Amigesic), Diflunisal (Dolobid), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac (Toradol) and Oxaprozin (Daypro).

Exambles of ErbB (e.g. ErbB2) receptor inhibitors include, but are not limited to, CP-724-714, CI-1033, (canertinib), Herceptin (trastuzumab), Omitarg (2C4, petuzumab), TAK-165, GW-572016 (lonafarnib), GW-282974, EKB-569, PI-166, AZD-8931 (sapitinib), dHER2 (HER2 Vaccine), APC8024 (HER2 Vaccine), anti-HER/2neu bispecific antibody, B7.her2IgG3, AS HER2 trifunctional bispecific antibodies, mAB AR-209 and mAB 2B-1.

Examples of Phosphoinositide 3-kinase inhibitor include, but are not limited to, Wortmannin, LY294002, hibiscone C, Idelalisib, Copanlisib, Duvelisib, Taselisib, Perifosine, Idelalisib, Buparlisib, Duvelisib, Alpelisib, Umbralisib, Copanlisib, PX-866, Dactolisib, CUDC-907, Voxtalisib (also known as SAR245409, XL765), CUDC-907, ME-401, IPI-549, SF1126, RP6530, INK1117, pictilisib, XL147 (also known as SAR245408), Palomid 529, GSK1059615, ZSTK474, PVVT33597, IC87114, TG100-115, CAL263, RP6503, PI-103, GNE-477, and AEZS-136.

Examples of alkylating agents include, but are not limited to, nitrogen mustard N-oxide, cyclophosphamide, ifosfamide, trofosfamide, Chlorambucil, melphalan, busulfan, mitobronitol, carboquone, thiotepa, ranimustine, nimustine, temozolomide, AMD-473, altretamine, AP-5280, apaziquone, brostallicin, bendamustine, carmustine, estramustine, fotemustine, glufosfamide, KW-2170, mafosfamide, and mitolactol, carmustine (BCNU), lomustine (CCNU), Busulfan, Treosulfan, Decarbazine and Temozolomide.

Examples of antimetabolites include but are not limited to, methotrexate, 6-mercaptopurine riboside, mercaptopurine, uracil analogues such as 5-fluorouracil (5-FU) alone or in combination with leucovorin, tegafur, UFT, doxifluridine, carmofur, cytarabine, cytarabine ocfosfate, enocitabine, S—I, Alimta (premetrexed disodium, LY231514, MTA), Gemzar (gemcitabine), fludarabine, 5-azacitidine, capecitabine, cladribine, clofarabine, decitabine, eflornithine, ethnylcytidine, cytosine arabinoside, hydroxyurea, TS-I, melphalan, nelarabine, nolatrexed, ocfosate, disodium premetrexed, pentostatin, pelitrexol, raltitrexed, triapine, trimetrexate, vidarabine, vincristine, vinorelbine, mycophenolic acid, tiazofurin, Ribavirin, EICAR, hydroxyurea and deferoxamine.

Examples of antibiotics include intercalating antibiotics but are not limited to, aclarubicin, actinomycins such as actinomycin D, amrubicin, annamycin, adriamycin, bleomycin a, bleomycin b, daunorubicin, doxorubicin, elsamitrucin, epirbucin, glarbuicin, idarubicin, mitomycin C, nemorubicin, neocarzinostatin, peplomycin, pirarubicin, rebeccamycin, stimalamer, streptozocin, valrubicin, zinostatin and combinations thereof.

Examples of topoisomerase inhibiting agents include, but are not limited to, one or more agents selected from the group consisting of aclarubicin, amonafide, belotecan, camptothecin, 10-hydroxycamptothecin, 9-aminocamptothecin, diflomotecan, irinotecan HCL (Camptosar), edotecarin, epirubicin (Ellence), etoposide, exatecan, gimatecan, lurtotecan, orathecin (Supergen), BN-80915, mitoxantrone, pirarbucin, pixantrone, rubitecan, sobuzoxane, SN-38, tafluposide and topotecan.

Examples of antibodies include, but are not limited to, Rituximab, Cetuximab, Bevacizumab, Trastuzumab, specific CD40 antibodies and specific IGFIR antibodies,

Examples of hormonal therapies include, but are not limited to, exemestane (Aromasin), leuprolide acetate, anastrozole (Arimidex), fosrelin (Zoladex), goserelin, doxercalciferol, fadrozole, formestane, tamoxifen citrate (tamoxifen), Casodex, Abarelix, Trelstar, finasteride, fulvestrant, toremifene, raloxifene, lasofoxifene, letrozole, flutamide, bicalutamide, megesterol, mifepristone, nilutamide, dexamethasone, predisone and other glucocorticoids.

Examples of retinoids/deltoids include, but are not limited to, seocalcitol (EB 1089, CB 1093), lexacalcitrol (KH 1060), fenretinide, Aliretinoin, Bexarotene and LGD-1550.

Examples of plant alkaloids include, but are not limited to, vincristine, vinblastine, vindesine and vinorelbine.

Examples of proteasome inhibitors include, but are not limited to, bortezomib (Velcade), MGI 32, NPI-0052 and PR-171.

Examples of immunologicals include, but are not limited to, interferons and numerous other immune enhancing agents. Interferons include interferon alpha, interferon alpha-2a, interferon, alpha-2b, interferon beta, interferon gamma-1a, interferon gamma-1 b (Actimmune), or interferon gamma-nl and combinations thereof. Other agents include filgrastim, lentinan, sizofilan, TheraCys, ubenimex, WF-10, aldesleukin, alemtuzumab, BAM-002, decarbazine, daclizumab, denileukin, gemtuzumab ozogamicin, ibritumomab, imiquimod, lenograstim, lentinan, melanoma vaccine (Corixa), molgramostim, OncoVAC-CL, sargaramostim, tasonermin, tecleukin, thymalasin, tositumomab, Virulizin, Z-100, epratuzumab, mitumomab, oregovomab, pemtumomab (Y-muHMFGI), Provenge (Dendreon), CTLA4 (cytotoxic lymphocyte antigen 4) antibodies and agents capable of blocking CTLA4 such as MDX-010.

Examples of biological response modifiers are agents that modify defense mechanisms of living organisms or biological responses, such as survival, growth, or differentiation of tissue cells to direct them to have anti-tumor activity. Such agents include krestin, lentinan, sizofrran, picibanil and ubenimex.

Examples of pyrimidine analogs include, but are not limited to, 5-Fluorouracil, Floxuridine, Doxifluridine, Ratitrexed, cytarabine (ara C), Cytosine arabinoside, Fludarabine, and Gemcitabine.

Examples of purine analogs include but are not limited to, Mercaptopurine and thioguanine.

Examples of antimitotic agents include, but are not limited to, ABT-751, paclitaxel, docetaxel, epothilone D (KOS-862) and ZK-EPO.

The antibodies or antigen binding fragments thereof of the present invention are also intended to be used as a radiosensitizer that enhances the efficacy of radiotherapy. Examples of radiotherapy include but are not limited to, external beam radiotherapy (XBRT), or teletherapy, brachtherapy or sealed source radiotherapy, unsealed source radiotherapy.

The antibodies or antigen binding fragments thereof of the present invention can also be used in combination with a different class of Bcl-2 inhibitors, such as ABT263 or ABT737.

According to some embodiments, the cytotoxic agent may be at least one of gemcitabine and abraxane.

According to yet another embodiment, the additional antibody or therapeutically fragment thereof may be oregovomab antibody B43.13, AR9.6 antibody, or combinations thereof.

According to an embodiment, the chemotherapy regimen may be Folfirinox.

In embodiments of the present invention, the tumor may be chosen from a pancreatic tumor, a gall bladder tumor, a gastric tumor, a colon tumor, an ovarian tumor, a breast tumor, and a liver tumor, and the method may be for the treatment of a cancer.

In another embodiment, there is disclosed a use of an antibody or an antigen binding fragment thereof that targets 0-glycan mucin-type glycoprotein MUC16, according to the present invention, or of a composition according to the present invention, for inhibiting tumor growth of a tumor expressing 0-glycan mucin-type glycoprotein MUC16 in a subject in need thereof.

In another embodiment, there is disclosed an antibody or an antigen binding fragment thereof that targets 0-glycan mucin-type glycoprotein MUC16, according to the present invention for use in inhibiting tumor growth of a tumor expressing 0-glycan mucin-type glycoprotein MUC16 in a subject in need thereof.

In another embodiment, there is disclosed an antibody or an antigen binding fragment thereof that targets 0-glycan mucin-type glycoprotein MUC16, according to the present invention for use in a method of inhibiting tumor growth of a tumor expressing 0-glycan mucin-type glycoprotein MUC16 in a subject in need thereof.

In another embodiment, there is disclosed a method of detection of a tumor expressing 0-glycan mucin-type glycoprotein MUC16 in a subject in need thereof, comprising administering to the subject an antibody or an antigen binding fragment thereof specific to 0-glycan mucin-type glycoprotein MUC16 according to the present invention and detecting the antibody or antigen binding fragment. According to an embodiment, the antibody or antigen binding fragment thereof may further comprise a detectable label, for example a fluorescent marker, a radioactive marker, an MRI contrast agent, or combinations thereof, as is known in the art.

The invention also encompasses nucleic acid vector comprising a nucleotide sequence encoding the antibody or antigen binding fragment thereof of the present invention, as well as cells comprising the nucleic acid vector, for expressing the antibody or antigen binding fragment thereof of the present invention, and cells for expressing the antibody or antigen binding fragment thereof of the present invention.

The present invention will be more readily understood by referring to the following examples which are given to illustrate the invention rather than to limit its scope.

### Example 1

### Anti-Her2/Neu Monoclonal Antibody Generation

The scFv C6MH3-B1 specific for HER2/neu has previously been used in the preparation of monoclonal antibodies, particularly IgE monoclonal antibodies (Daniels, T. R. et al. Cancer Immunol Immunother, 2012 July; 61(7):991-1003). However, this antibody has failed to become useful as a therapeutic because low production yield prevent it from being made in sufficient quantities and in a form that would be useable for therapy. This could be caused by numerous issues in addition to self-aggregation, oxidation, deamination and isomerization of amino acid chains.

The sequence of the anti-Her2/Neu IgE antibody derived from the C6MH3-B1 scFv comprises a full-length Lambda isotype light chain and a full length IgE heavy chain. As a first step in modifying the structure to improve production yield, the sequence of the entire IgE heavy chain (SEQ ID NO: 13), and particularly the heavy chain variable region from FR1 to FR4 (SEQ ID NO: 14) was point mutated at numerous amino acid residues. The wild type and the mutant variant sequence of the antibody C6MH3-B1 were transiently expressed in Chinese Hamster Ovary cells (CHOK1SV GS-KO) using small scale transient expression, to screen for both improved expression and binding to the antigen. Mutations that caused a decrease in or no improvement on the level of expression were discarded in a first pass. Those that decreased binding of the antibodies to the antigen were also discarded in a first pass. The selected variants were recloned by limiting dilutions to ensure their monoclonality, and sequenced. From the numerous mutants identified, mutations in the CDR2 region were identified and selected for further characterization. Three mutations of the first aspartic acid (D) residue of CDR H2 were identified: D to E (variant 1), D to Q (variant 2), and D to S (variant 3). Two mutations of the first serine (S) residue of CDR H2 were identified: S to T (variant 4) and S to I (variant 5).

Antibodies were produced for the wild type and the mutant variants 1 to 5 from transient expression in Chinese Hamster Ovary cells (CHOK1SV GS-KO) using small scale transient expression HiTrap® LambdaFabSelect resin (Sigma®) and buffer exchanged into PBS containing 3 mM EDTA.

### Example 2

### Anti-Her2/Neu Monoclonal Antibody Yield Measurement

During extraction with the LambdaFabSelect resin, an estimation of the titre of antibody was made from the obtained chromatograms (no shown). The results are summarized in Table 1, below:

TABLE 1
Estimation of titre from LambdaFabSelect chromatogram

| | Totl | Extinction | Calculated | Estimated |
|---|---|---|---|---|
| | peak area | coefficient | yield | Titre |
| Product | (mL mAU) | (mg mL⁻¹ cm⁻⁻¹) | (mg) | (mg/L) |
| Parental | 376 | 1.77 | 1.061 | 5.31 |
| Variant 1 | 1492 | 1.77 | 4.214 | 21.07 |
| Variant 2 | 1180 | 1.77 | 3.332 | 16.66 |
| Variant 3 | 1074 | 1.77 | 3.033 | 15.17 |
| Variant 4 | 399 | 1.77 | 1.127 | 5.64 |
| Variant 5 | 1695 | 1.77 | 4.787 | 23.94 |

The results show that unexpectedly, cells expressing the variants 1 to 3 and 5 were able to increase expression and provide higher yields of the antibodies than the parental C6MH3-B1 IgE antibody. Variant 4 performed only slightly better than the parental antibody.

### Example 3

### Anti-Her2/Neu Monoclonal Antibody Purification and Aggregation

Pursuant to elution of the purified antibodies from the LambdaFabSelect resin, they were subjected to size exclusion chromatography (SEC) using a Superdex® S75 10/300 GL column. The results are summarized in Table 2, below:

TABLE 2
SEC analysis of the eluted fractions from
LambdaFabSelect Column containing antibodies

| Product | % Aggregate | % Monomer | % LMW |
|---|---|---|---|
| Parental, Peak 1 | 4.10 | 95.81 | 0.05 |
| Var1, Peak 1 | — | 99.99 | 0.01 |
| Var2, Peak 1 | — | 100.00 | — |
| Var2, Peak 2 | 31.03 | 55.61 | 13.36 |
| Var3, Peak 1 | 52.23 | 32.80 | 14.97 |
| Var4, Peak 1 | — | 100.00 | — |
| Var4, Peak 2 | — | 100.00 | — |
| Var5, Peak 1 | — | 99.89 | 0.11 |
| Var5, Peak 2 | — | 100.00 | — |

The results show that unexpectedly, variants 1, 4 and 5 eluted as monomeric antibodies and showed no aggregates and no or only small amount of low molecular weight (LMW) products. Variant 2 eluted as two peaks, the first of which unexpectedly comprised of monomers, while the second comprised a significant portion of undesirable aggregates (31.03%) and LMW products (13.36). Variant 3 eluted as one peak comprised of undesirable aggregates (52.23%), monomers (32.80) and low molecular weight (LMVV) products (14.97%). Therefore, variants 1, 2, 4 and 5 showed an unexpected improvement: variants 1, 4 and 5 eluted as a single peak with no aggregation, while variant 2 could be purified in unaggregated form from the first peak.

### Example 4

### Anti-Her2/Neu Monoclonal Antibody Affinity

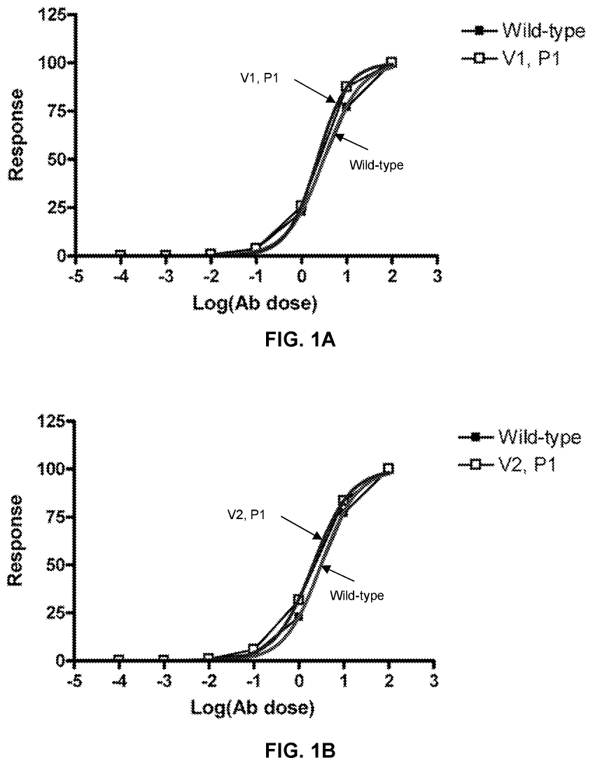

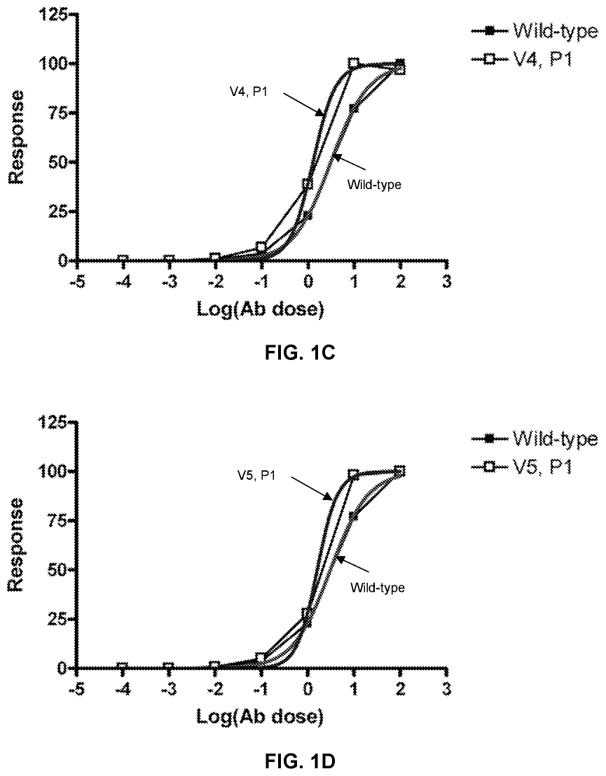

Next, the binding of variants 1, 2, 4 and 5 was compared to that of the parental antibody by flow cytometry. D2F2/E2 cells [Wei et al. Int J Cancer, 1999 May 31; 81(5):748-54] expressing human Her2/Neu were cultured. Cells were detached from tissue culture dishes using 0.5 mM EDTA and incubated with varying concentrations of the various antibodies for 2 h on ice. Cells were then washed in buffer (2 mM EDTA, 0.5% BSA in PBS) and binding was detected using an anti-human κ fluorescein isothiocyanate (FITC)-conjugated secondary antibody (BD Biosciences, San Jose, Calif.). Cells were also stained with an anti-FIERI phycoerythrin (PE)-conjugate (eBioscience, San Diego, Calif.) to ensure expression of the receptor on the surface of the cells. All cells were washed, fixed with 2% paraformaldehyde in PBS, and analyzed on a Becton-Dickinson FACScan Analytic Flow Cytometer. Now referring to FIG. 1, the results show that unexpectedly, despite the mutations introduced into the CDR H2 region, variants 1, 2, 4 and 5 are all able to bind to Her2/Neu with affinities that are at least as good as that of the wild-type parental antibody. Indeed, variants 1 and 2 have affinities that are close to, or even a little better than the parental antibody (FIGS. 1A and 1B). Variants 4 and 5, on the other hand, appear to display affinities that are unexpectedly improved over that of the wild-type parental antibody (FIGS. 1C and 1D).

While preferred embodiments have been described above and illustrated in the accompanying drawings, it will be evident to those skilled in the art that modifications may be made without departing from this disclosure. Such modifications are considered as possible variants comprised in the scope of the disclosure.

SEQUENCE TABLE

| SEQ ID NO | Sequence | Description |
|---|---|---|
| SEQ ID NO: 1 | GYSFTSYW | CDR H1 |
| SEQ ID NO: 2 | IYPGX₁X₂DT, where X₁ is D, E, or Q, X₂ is S, | CDR H2 |
| | I, or T, and wherein when X₁ is D, X₂ is | |
| | different than S | |
| SEQ ID NO: 3 | ARHDVGYCTDRTCAKWPEY | CDR H3 |
| SEQ ID NO: 4 | SSNIGNNY | CDR L1 |
| SEQ ID NO: 5 | DHT | CDR L2 |
| SEQ ID NO: 6 | ASWDYTLSGWV | CDR L3 |
| SEQ ID NO: 7 | AAGCTTGCCGCCACCATGGAATGGTCCTGGGTGTTCCTGTTC | Anti-Her2/Neu |
| | TTCCTGTCCGTGACCACCGGCGTGCACTCTCAGGTTCAGTTG | IgE antibody |
| | GTTCAGTCTGGCGCCGAAGTGAAGAAGCCTGGCGAGTCCCTG | Full length |
| | AAGATCTCCTGCAAAGGCTCCGGCTACTCCTTCACCTCCTAC | heavy chain |
| | TGGATCGCCTGGGTCCGACAGATGCCTGGAAAGGGCCTCGAG | 0 |
| | TACATGGGCCTGATCTACCCTGGCGACTCCGATACCAAGTAC | |
| | TCCCCTAGCTTCCAGGGCCAAGTGACCATCTCCGTGGACAAG | |
| | TCTGTGTCCACCGCCTACCTGCAGTGGTCCTCTCTGAAGCCT | |
| | TCTGACTCCGCCGTGTACTTCTGCGCCAGGCACGATGTGGGC | |
| | TACTGCACCGATAGAACCTGCGCCAAGTGGCCCGAGTACTTT | |
| | CAGCATTGGGGCCAGGGCACACTGGTCACCGTTTCTTCCGCC | |
| | TCTACACAGTCCCCTAGCGTGTTCCCTCTGACCAGATGCTGC | |
| | AAGAACATCCCCTCCAACGCCACCTCTGTGACCCTGGGATGT | |
| | CTGGCCACCGGCTATTTTCCCGAGCCTGTGATGGTCACCTGG | |
| | GACACCGGATCTCTGAACGGCACCACCATGACACTGCCTGCC | |
| | ACCACACTGACCCTGTCTGGACACTACGCTACCATCAGCCTG | |
| | CTGACAGTGTCTGGCGCTTGGGCCAAGCAGATGTTCACCTGT | |
| | AGAGTGGCTCACACCCCTTCCAGCACCGACTGGGTCGACAAC | |
| | AAGACCTTCTCCGTGTGCTCCCGGGACTTCACCCCTCCTACA | |
| | GTGAAGATCCTGCAGTCCTCTTGTGATGGCGGCGGACACTTC | |
| | CCACCTACAATCCAGCTGCTGTGCCTGGTGTCTGGCTATACC | |
| | CCTGGCACCATCAACATCACCTGGCTGGAAGATGGACAAGTG | |
| | ATGGACGTGGACCTGAGCACCGCCAGCACAACACAAGAAGGC | |
| | GAGCTGGCTTCTACCCAGTCCGAGCTGACACTGTCCCAGAAG | |
| | CACTGGCTGTCCGACCGGACCTACACCTGTCAAGTGACCTAC | |
| | CAGGGCCACACCTTCGAGGACTCCACCAAGAAGTGCGCCGAC | |
| | AGCAATCCTAGAGGCGTGTCCGCTTACCTGAGCCGGCCTTCT | |
| | CCTTTCGACCTGTTCATCCGGAAGTCTCCCACCATCACATGT | |
| | CTGGTGGTGGATCTGGCCCCTTCCAAGGGCACCGTGAATCTG | |
| | ACCTGGTCTAGAGCCTCTGGCAAGCCCGTGAACCACAGCACC | |
| | CGGAAAGAGGAAAAGCAGCGCAACGGCACCCTGACCGTGACC | |
| | TCTACACTGCCAGTGGGCACCAGAGATTGGATCGAGGGCGAG | |
| | ACATACCAGTGCAGAGTGACCCATCCTCATCTGCCTCGGGCT | |
| | CTGATGCGGTCTACCACCAAGACCTCTGGACCTAGAGCTGCC | |
| | CCTGAGGTGTACGCCTTTGCTACACCTGAGTGGCCTGGCTCC | |
| | AGAGACAAGAGAACCCTGGCCTGCCTGATCCAGAACTTCATG | |
| | CCCGAGGACATCAGCGTGCAGTGGCTGCACAATGAGGTGCAG | |
| | CTGCCTGACGCTCGGCACTCTACAACCCAGCCTAGAAAGACC | |
| | AAAGGCAGCGGCTTCTTCGTGTTCTCTCGGCTGGAAGTGACC | |
| | AGAGCCGAGTGGGAGCAGAAGGACGAGTTCATCTGCAGAGCC | |
| | GTGCACGAGGCTGCCTCTCCATCTCAGACAGTTCAGAGGGCC | |
| | GTGTCTGTGAACCCCGGCAAGTAATGAATTC | |
| SEQ ID NO: 8 | AAGCTTGCCGCCACCATGGAATGGTCCTGGGTGTTCCTGTTC | Anti-Her2/Neu |
| | TTCCTGTCCGTGACCACCGGCGTGCACTCTCAGGTTCAGTTG | IgE antibody |
| | GTTCAGTCTGGCGCCGAAGTGAAGAAGCCTGGCGAGTCCCTG | Full length |
| | AAGATCTCCTGCAAAGGCTCCGGCTACTCCTTCACCTCCTAC | heavy chain |
| | TGGATCGCCTGGGTCCGACAGATGCCTGGAAAGGGCCTCGAG | 1 |
| | TACATGGGCCTGATCTATCCCGGCGAGTCCGATACCAAGTAC | |
| | TCCCCTAGCTTCCAGGGCCAAGTGACCATCTCCGTGGACAAG | |
| | TCTGTGTCCACCGCCTACCTGCAGTGGTCCTCTCTGAAGCCT | |
| | TCTGACTCCGCCGTGTACTTCTGCGCCAGGCACGATGTGGGC | |
| | TACTGCACCGATAGAACCTGCGCCAAGTGGCCCGAGTACTTT | |
| | CAGCATTGGGGCCAGGGCACACTGGTCACCGTTTCTTCCGCC | |
| | TCTACACAGTCCCCTAGCGTGTTCCCTCTGACCAGATGCTGC | |
| | AAGAACATCCCCTCCAACGCCACCTCTGTGACCCTGGGATGT | |
| | CTGGCCACCGGCTATTTTCCCGAGCCTGTGATGGTCACCTGG | |
| | GACACCGGATCTCTGAACGGCACCACCATGACACTGCCTGCC | |
| | ACCACACTGACCCTGTCTGGACACTACGCTACCATCAGCCTG | |
| | CTGACAGTGTCTGGCGCTTGGGCCAAGCAGATGTTCACCTGT | |
| | AGAGTGGCTCACACCCCTTCCAGCACCGACTGGGTCGACAAC | |
| | AAGACCTTCTCCGTGTGCTCCCGGGACTTCACCCCTCCTACA | |
| | GTGAAGATCCTGCAGTCCTCTTGTGATGGCGGCGGACACTTC | |
| | CCACCTACAATCCAGCTGCTGTGCCTGGTGTCTGGCTATACC | |
| | CCTGGCACCATCAACATCACCTGGCTGGAAGATGGACAAGTG | |
| | ATGGACGTGGACCTGAGCACCGCCAGCACAACACAAGAAGGC | |
| | GAGCTGGCTTCTACCCAGTCCGAGCTGACACTGTCCCAGAAG | |
| | CACTGGCTGTCCGACCGGACCTACACCTGTCAAGTGACCTAC | |
| | CAGGGCCACACCTTCGAGGACTCCACCAAGAAGTGCGCCGAC | |
| | AGCAATCCTAGAGGCGTGTCCGCTTACCTGAGCCGGCCTTCT | |
| | CCTTTCGACCTGTTCATCCGGAAGTCTCCCACCATCACATGT | |
| | CTGGTGGTGGATCTGGCCCCTTCCAAGGGCACCGTGAATCTG | |
| | ACCTGGTCTAGAGCCTCTGGCAAGCCCGTGAACCACAGCACC | |
| | CGGAAAGAGGAAAAGCAGCGCAACGGCACCCTGACCGTGACC | |
| | TCTACACTGCCAGTGGGCACCAGAGATTGGATCGAGGGCGAG | |
| | ACATACCAGTGCAGAGTGACCCATCCTCATCTGCCTCGGGCT | |
| | CTGATGCGGTCTACCACCAAGACCTCTGGACCTAGAGCTGCC | |
| | CCTGAGGTGTACGCCTTTGCTACACCTGAGTGGCCTGGCTCC | |
| | AGAGACAAGAGAACCCTGGCCTGCCTGATCCAGAACTTCATG | |
| | CCCGAGGACATCAGCGTGCAGTGGCTGCACAATGAGGTGCAG | |
| | CTGCCTGACGCTCGGCACTCTACAACCCAGCCTAGAAAGACC | |
| | AAAGGCAGCGGCTTCTTCGTGTTCTCTCGGCTGGAAGTGACC | |
| | AGAGCCGAGTGGGAGCAGAAGGACGAGTTCATCTGCAGAGCC | |
| | GTGCACGAGGCTGCCTCTCCATCTCAGACAGTTCAGAGGGCC | |
| | GTGTCTGTGAACCCCGGCAAGTAATGAATTC | |
| SEQ ID NO: 9 | AAGCTTGCCGCCACCATGGAATGGTCCTGGGTGTTCCTGTTC | Anti-Her2/Neu |
| | TTCCTGTCCGTGACCACCGGCGTGCACTCTCAGGTTCAGTTG | IgE antibody |
| | GTTCAGTCTGGCGCCGAAGTGAAGAAGCCTGGCGAGTCCCTG | Full length |
| | AAGATCTCCTGCAAAGGCTCCGGCTACTCCTTCACCTCCTAC | heavy chain |
| | TGGATCGCCTGGGTCCGACAGATGCCTGGAAAGGGCCTCGAG | 2 |
| | TACATGGGCCTGATCTACCCTGGCCAGTCCGATACCAAGTAC | |
| | TCCCCTAGCTTCCAGGGCCAAGTGACCATCTCCGTGGACAAG | |
| | TCTGTGTCCACCGCCTACCTGCAGTGGTCCTCTCTGAAGCCT | |
| | TCTGACTCCGCCGTGTACTTCTGCGCCAGGCACGATGTGGGC | |
| | TACTGCACCGATAGAACCTGCGCCAAGTGGCCCGAGTACTTT | |
| | CAGCATTGGGGCCAGGGCACACTGGTCACCGTTTCTTCCGCC | |
| | TCTACACAGTCCCCTAGCGTGTTCCCTCTGACCAGATGCTGC | |
| | AAGAACATCCCCTCCAACGCCACCTCTGTGACCCTGGGATGT | |
| | CTGGCCACCGGCTATTTTCCCGAGCCTGTGATGGTCACCTGG | |
| | GACACCGGATCTCTGAACGGCACCACCATGACACTGCCTGCC | |
| | ACCACACTGACCCTGTCTGGACACTACGCTACCATCAGCCTG | |
| | CTGACAGTGTCTGGCGCTTGGGCCAAGCAGATGTTCACCTGT | |
| | AGAGTGGCTCACACCCCTTCCAGCACCGACTGGGTCGACAAC | |
| | AAGACCTTCTCCGTGTGCTCCCGGGACTTCACCCCTCCTACA | |
| | GTGAAGATCCTGCAGTCCTCTTGTGATGGCGGCGGACACTTC | |
| | CCACCTACAATCCAGCTGCTGTGCCTGGTGTCTGGCTATACC | |
| | CCTGGCACCATCAACATCACCTGGCTGGAAGATGGACAAGTG | |
| | ATGGACGTGGACCTGAGCACCGCCAGCACAACACAAGAAGGC | |
| | GAGCTGGCTTCTACCCAGTCCGAGCTGACACTGTCCCAGAAG | |
| | CACTGGCTGTCCGACCGGACCTACACCTGTCAAGTGACCTAC | |
| | CAGGGCCACACCTTCGAGGACTCCACCAAGAAGTGCGCCGAC | |
| | AGCAATCCTAGAGGCGTGTCCGCTTACCTGAGCCGGCCTTCT | |
| | CCTTTCGACCTGTTCATCCGGAAGTCTCCCACCATCACATGT | |
| | CTGGTGGTGGATCTGGCCCCTTCCAAGGGCACCGTGAATCTG | |
| | ACCTGGTCTAGAGCCTCTGGCAAGCCCGTGAACCACAGCACC | |
| | CGGAAAGAGGAAAAGCAGCGCAACGGCACCCTGACCGTGACC | |
| | TCTACACTGCCAGTGGGCACCAGAGATTGGATCGAGGGCGAG | |
| | ACATACCAGTGCAGAGTGACCCATCCTCATCTGCCTCGGGCT | |
| | CTGATGCGGTCTACCACCAAGACCTCTGGACCTAGAGCTGCC | |
| | CCTGAGGTGTACGCCTTTGCTACACCTGAGTGGCCTGGCTCC | |
| | AGAGACAAGAGAACCCTGGCCTGCCTGATCCAGAACTTCATG | |
| | CCCGAGGACATCAGCGTGCAGTGGCTGCACAATGAGGTGCAG | |
| | CTGCCTGACGCTCGGCACTCTACAACCCAGCCTAGAAAGACC | |
| | AAAGGCAGCGGCTTCTTCGTGTTCTCTCGGCTGGAAGTGACC | |
| | AGAGCCGAGTGGGAGCAGAAGGACGAGTTCATCTGCAGAGCC | |
| | GTGCACGAGGCTGCCTCTCCATCTCAGACAGTTCAGAGGGCC | |
| | GTGTCTGTGAACCCCGGCAAGTAATGAATTC | |
| SEQ ID NO: 10 | AAGCTTGCCGCCACCATGGAATGGTCCTGGGTGTTCCTGTTC | Anti-Her2/Neu |
| | TTCCTGTCCGTGACCACCGGCGTGCACTCTCAGGTTCAGTTG | IgE antibody |
| | GTTCAGTCTGGCGCCGAAGTGAAGAAGCCTGGCGAGTCCCTG | Full length |
| | AAGATCTCCTGCAAAGGCTCCGGCTACTCCTTCACCTCCTAC | heavy chain |
| | TGGATCGCCTGGGTCCGACAGATGCCTGGAAAGGGCCTCGAG | 3 |
| | TACATGGGCCTGATCTACCCTGGCTCCTCCGATACCAAGTAC | |
| | TCCCCTAGCTTCCAGGGCCAAGTGACCATCTCCGTGGACAAG | |
| | TCTGTGTCCACCGCCTACCTGCAGTGGTCCTCTCTGAAGCCT | |
| | TCTGACTCCGCCGTGTACTTCTGCGCCAGGCACGATGTGGGC | |
| | TACTGCACCGATAGAACCTGCGCCAAGTGGCCCGAGTACTTT | |
| | CAGCATTGGGGCCAGGGCACACTGGTCACCGTTTCTTCCGCC | |
| | TCTACACAGTCCCCTAGCGTGTTCCCTCTGACCAGATGCTGC | |
| | AAGAACATCCCCTCCAACGCCACCTCTGTGACCCTGGGATGT | |
| | CTGGCCACCGGCTATTTTCCCGAGCCTGTGATGGTCACCTGG | |
| | GACACCGGATCTCTGAACGGCACCACCATGACACTGCCTGCC | |
| | ACCACACTGACCCTGTCTGGACACTACGCTACCATCAGCCTG | |
| | CTGACAGTGTCTGGCGCTTGGGCCAAGCAGATGTTCACCTGT | |
| | AGAGTGGCTCACACCCCTTCCAGCACCGACTGGGTCGACAAC | |
| | AAGACCTTCTCCGTGTGCTCCCGGGACTTCACCCCTCCTACA | |
| | GTGAAGATCCTGCAGTCCTCTTGTGATGGCGGCGGACACTTC | |
| | CCACCTACAATCCAGCTGCTGTGCCTGGTGTCTGGCTATACC | |
| | CCTGGCACCATCAACATCACCTGGCTGGAAGATGGACAAGTG | |
| | ATGGACGTGGACCTGAGCACCGCCAGCACAACACAAGAAGGC | |
| | GAGCTGGCTTCTACCCAGTCCGAGCTGACACTGTCCCAGAAG | |
| | CACTGGCTGTCCGACCGGACCTACACCTGTCAAGTGACCTAC | |
| | CAGGGCCACACCTTCGAGGACTCCACCAAGAAGTGCGCCGAC | |
| | AGCAATCCTAGAGGCGTGTCCGCTTACCTGAGCCGGCCTTCT | |
| | CCTTTCGACCTGTTCATCCGGAAGTCTCCCACCATCACATGT | |
| | CTGGTGGTGGATCTGGCCCCTTCCAAGGGCACCGTGAATCTG | |
| | ACCTGGTCTAGAGCCTCTGGCAAGCCCGTGAACCACAGCACC | |
| | CGGAAAGAGGAAAAGCAGCGCAACGGCACCCTGACCGTGACC | |
| | TCTACACTGCCAGTGGGCACCAGAGATTGGATCGAGGGCGAG | |
| | ACATACCAGTGCAGAGTGACCCATCCTCATCTGCCTCGGGCT | |
| | CTGATGCGGTCTACCACCAAGACCTCTGGACCTAGAGCTGCC | |
| | CCTGAGGTGTACGCCTTTGCTACACCTGAGTGGCCTGGCTCC | |
| | AGAGACAAGAGAACCCTGGCCTGCCTGATCCAGAACTTCATG | |
| | CCCGAGGACATCAGCGTGCAGTGGCTGCACAATGAGGTGCAG | |
| | CTGCCTGACGCTCGGCACTCTACAACCCAGCCTAGAAAGACC | |
| | AAAGGCAGCGGCTTCTTCGTGTTCTCTCGGCTGGAAGTGACC | |
| | AGAGCCGAGTGGGAGCAGAAGGACGAGTTCATCTGCAGAGCC | |
| | GTGCACGAGGCTGCCTCTCCATCTCAGACAGTTCAGAGGGCC | |
| | GTGTCTGTGAACCCCGGCAAGTAATGAATTC | |
| SEQ ID NO: 11 | AAGCTTGCCGCCACCATGGAATGGTCCTGGGTGTTCCTGTTC | Anti-Her2/Neu |
| | TTCCTGTCCGTGACCACCGGCGTGCACTCTCAGGTTCAGTTG | IgE antibody |
| | GTTCAGTCTGGCGCCGAAGTGAAGAAGCCTGGCGAGTCCCTG | Full length |
| | AAGATCTCCTGCAAAGGCTCCGGCTACTCCTTCACCTCCTAC | heavy chain |
| | TGGATCGCCTGGGTCCGACAGATGCCTGGAAAGGGCCTCGAG | 4 |
| | TACATGGGCCTGATCTACCCTGGCGACACCGATACCAAGTAC | |
| | AGCCCCAGCTTTCAGGGCCAAGTGACCATCTCCGTGGACAAG | |
| | TCTGTGTCCACCGCCTACCTGCAGTGGTCCTCTCTGAAGCCT | |
| | TCTGACTCCGCCGTGTACTTCTGCGCCAGGCACGATGTGGGC | |
| | TACTGCACCGATAGAACCTGCGCCAAGTGGCCCGAGTACTTT | |
| | CAGCATTGGGGCCAGGGCACACTGGTCACCGTTTCTTCCGCC | |
| | TCTACACAGTCCCCTAGCGTGTTCCCTCTGACCAGATGCTGC | |
| | AAGAACATCCCCTCCAACGCCACCTCTGTGACCCTGGGATGT | |
| | CTGGCCACCGGCTATTTTCCCGAGCCTGTGATGGTCACCTGG | |
| | GACACCGGATCTCTGAACGGCACCACCATGACACTGCCTGCC | |
| | ACCACACTGACCCTGTCTGGACACTACGCTACCATCAGCCTG | |
| | CTGACAGTGTCTGGCGCTTGGGCCAAGCAGATGTTCACCTGT | |
| | AGAGTGGCTCACACCCCTTCCAGCACCGACTGGGTCGACAAC | |
| | AAGACCTTCTCCGTGTGCTCCCGGGACTTCACCCCTCCTACA | |
| | GTGAAGATCCTGCAGTCCTCTTGTGATGGCGGCGGACACTTC | |
| | CCACCTACAATCCAGCTGCTGTGCCTGGTGTCTGGCTATACC | |
| | CCTGGCACCATCAACATCACCTGGCTGGAAGATGGACAAGTG | |
| | ATGGACGTGGACCTGAGCACCGCCAGCACAACACAAGAAGGC | |
| | GAGCTGGCTTCTACCCAGTCCGAGCTGACACTGTCCCAGAAG | |
| | CACTGGCTGTCCGACCGGACCTACACCTGTCAAGTGACCTAC | |
| | CAGGGCCACACCTTCGAGGACTCCACCAAGAAGTGCGCCGAC | |
| | AGCAATCCTAGAGGCGTGTCCGCTTACCTGAGCCGGCCTTCT | |
| | CCTTTCGACCTGTTCATCCGGAAGTCTCCCACCATCACATGT | |
| | CTGGTGGTGGATCTGGCCCCTTCCAAGGGCACCGTGAATCTG | |
| | ACCTGGTCTAGAGCCTCTGGCAAGCCCGTGAACCACAGCACC | |
| | CGGAAAGAGGAAAAGCAGCGCAACGGCACCCTGACCGTGACC | |
| | TCTACACTGCCAGTGGGCACCAGAGATTGGATCGAGGGCGAG | |
| | ACATACCAGTGCAGAGTGACCCATCCTCATCTGCCTCGGGCT | |
| | CTGATGCGGTCTACCACCAAGACCTCTGGACCTAGAGCTGCC | |
| | CCTGAGGTGTACGCCTTTGCTACACCTGAGTGGCCTGGCTCC | |
| | AGAGACAAGAGAACCCTGGCCTGCCTGATCCAGAACTTCATG | |
| | CCCGAGGACATCAGCGTGCAGTGGCTGCACAATGAGGTGCAG | |
| | CTGCCTGACGCTCGGCACTCTACAACCCAGCCTAGAAAGACC | |
| | AAAGGCAGCGGCTTCTTCGTGTTCTCTCGGCTGGAAGTGACC | |
| | AGAGCCGAGTGGGAGCAGAAGGACGAGTTCATCTGCAGAGCC | |
| | GTGCACGAGGCTGCCTCTCCATCTCAGACAGTTCAGAGGGCC | |
| | GTGTCTGTGAACCCCGGCAAGTAATGAATTC | |
| SEQ ID NO: 12 | AAGCTTGCCGCCACCATGGAATGGTCCTGGGTGTTCCTGTTC | Anti-Her2/Neu |
| | TTCCTGTCCGTGACCACCGGCGTGCACTCTCAGGTTCAGTTG | IgE antibody |
| | GTTCAGTCTGGCGCCGAAGTGAAGAAGCCTGGCGAGTCCCTG | Full length |
| | AAGATCTCCTGCAAAGGCTCCGGCTACTCCTTCACCTCCTAC | heavy chain |
| | TGGATCGCCTGGGTCCGACAGATGCCTGGAAAGGGCCTCGAG | 5 |
| | TACATGGGCCTGATCTACCCTGGCGACATCGATACCAAGTAC | |
| | AGCCCCAGCTTCCAGGGCCAAGTGACCATCTCCGTGGACAAG | |
| | TCTGTGTCCACCGCCTACCTGCAGTGGTCCTCTCTGAAGCCT | |
| | TCTGACTCCGCCGTGTACTTCTGCGCCAGGCACGATGTGGGC | |
| | TACTGCACCGATAGAACCTGCGCCAAGTGGCCCGAGTACTTT | |
| | CAGCATTGGGGCCAGGGCACACTGGTCACCGTTTCTTCCGCC | |
| | TCTACACAGTCCCCTAGCGTGTTCCCTCTGACCAGATGCTGC | |
| | AAGAACATCCCCTCCAACGCCACCTCTGTGACCCTGGGATGT | |
| | CTGGCCACCGGCTATTTTCCCGAGCCTGTGATGGTCACCTGG | |
| | GACACCGGATCTCTGAACGGCACCACCATGACACTGCCTGCC | |
| | ACCACACTGACCCTGTCTGGACACTACGCTACCATCAGCCTG | |
| | CTGACAGTGTCTGGCGCTTGGGCCAAGCAGATGTTCACCTGT | |
| | AGAGTGGCTCACACCCCTTCCAGCACCGACTGGGTCGACAAC | |
| | AAGACCTTCTCCGTGTGCTCCCGGGACTTCACCCCTCCTACA | |
| | GTGAAGATCCTGCAGTCCTCTTGTGATGGCGGCGGACACTTC | |
| | CCACCTACAATCCAGCTGCTGTGCCTGGTGTCTGGCTATACC | |
| | CCTGGCACCATCAACATCACCTGGCTGGAAGATGGACAAGTG | |
| | ATGGACGTGGACCTGAGCACCGCCAGCACAACACAAGAAGGC | |
| | GAGCTGGCTTCTACCCAGTCCGAGCTGACACTGTCCCAGAAG | |
| | CACTGGCTGTCCGACCGGACCTACACCTGTCAAGTGACCTAC | |
| | CAGGGCCACACCTTCGAGGACTCCACCAAGAAGTGCGCCGAC | |
| | AGCAATCCTAGAGGCGTGTCCGCTTACCTGAGCCGGCCTTCT | |
| | CCTTTCGACCTGTTCATCCGGAAGTCTCCCACCATCACATGT | |
| | CTGGTGGTGGATCTGGCCCCTTCCAAGGGCACCGTGAATCTG | |
| | ACCTGGTCTAGAGCCTCTGGCAAGCCCGTGAACCACAGCACC | |
| | CGGAAAGAGGAAAAGCAGCGCAACGGCACCCTGACCGTGACC | |
| | TCTACACTGCCAGTGGGCACCAGAGATTGGATCGAGGGCGAG | |
| | ACATACCAGTGCAGAGTGACCCATCCTCATCTGCCTCGGGCT | |
| | CTGATGCGGTCTACCACCAAGACCTCTGGACCTAGAGCTGCC | |
| | CCTGAGGTGTACGCCTTTGCTACACCTGAGTGGCCTGGCTCC | |
| | AGAGACAAGAGAACCCTGGCCTGCCTGATCCAGAACTTCATG | |
| | CCCGAGGACATCAGCGTGCAGTGGCTGCACAATGAGGTGCAG | |
| | CTGCCTGACGCTCGGCACTCTACAACCCAGCCTAGAAAGACC | |
| | AAAGGCAGCGGCTTCTTCGTGTTCTCTCGGCTGGAAGTGACC | |
| | AGAGCCGAGTGGGAGCAGAAGGACGAGTTCATCTGCAGAGCC | |
| | GTGCACGAGGCTGCCTCTCCATCTCAGACAGTTCAGAGGGCC | |
| | GTGTCTGTGAACCCCGGCAAGTAATGAATTC | |
| SEQ ID NO: 13 | KLAATMEWSWVFLFFLSVTTGVHSQVQLVQSGAEVKKPGESL | Amino Acid |
| | KISCKGSGYSFTSYWIAWVRQMPGKGLEYMGLIYPGDSDTKY | Sequence of |
| | SPSFQGQVTISVDKSVSTAYLQWSSLKPSDSAVYFCARHDVG | anti-Her2/Neu |
| | YCTDRTCAKWPEYFQHWGQGTLVTVSSASTQSPSVFPLTRCC | IgE antibody |
| | KNIPSNATSVTLGCLATGYFPEPVMVTWDTGSLNGTTMTLPA | Full length |
| | TTLTLSGHYATISLLTVSGAWAKQMFTCRVAHTPSSTDWVDN | heavy chain |
| | KTFSVCSRDFTPPTVKILQSSCDGGGHFPPTIQLLCLVSGYT | 0 |
| | PGTINITWLEDGQVMDVDLSTASTTQEGELASTQSELTLSQK | |
| | HWLSDRTYTCQVTYQGHTFEDSTKKCADSNPRGVSAYLSRPS | |
| | PFDLFIRKSPTITCLVVDLAPSKGTVNLTWSRASGKPVNHST | |
| | RKEEKQRNGTLTVTSTLPVGTRDWIEGETYQCRVTHPHLPRA | |
| | LMRSTTKTSGPRAAPEVYAFATPEWPGSRDKRTLACLIQNFM | |
| | PEDISVQWLHNEVQLPDARHSTTQPRKTKGSGFFVFSRLEVT | |
| | RAEWEQKDEFICRAVHEAASPSQTVQRAVSVNPGK | |
| SEQ ID NO: 14 | VQLVQSGAEVKKPGESLKISCKGSGYSFTSYWIAWVRQMPGK | Amino Acid |
| | GLEYMGLIYPGDSDTKYSPSFQGQVTISVDKSVSTAYLQWSS | Sequence of |
| | LKPSDSAVYFCARHDVGYCTDRTCAKWPEYFQHWGQGTLVTV | anti-Her2/Neu |
| | | IgE antibody 0 |
| | | heavy chain |
| | | variable region |
| | | from FR1 to FR4 |
| SEQ ID NO: 15 | VQLVQSGAEVKKPGESLKISCKGS | Amino Acid |
| | | Sequence of |
| | | anti-Her2/Neu |
| | | IgE antibody 0 |
| | | heavy chain |
| | | variable region |
| | | FR1 |
| SEQ ID NO: 16 | IAWVRQMPGKGLEYMGL | Amino Acid |
| | | Sequence of |
| | | anti-Her2/Neu |
| | | IgE antibody 0 |
| | | heavy chain |
| | | variable region |
| | | FR2 |
| SEQ ID NO: 17 | KYSPSFQGQVTISVDKSVSTAYLQWSSLKPSDSAVYFC | Amino Acid |
| | | Sequence of |
| | | anti-Her2/Neu |
| | | IgE antibody 0 |
| | | heavy chain |
| | | variable region |
| | | FR3 |
| SEQ ID NO: 18 | WGQGTLVTV | Amino Acid |
| | | Sequence of |
| | | anti-Her2/Neu |
| | | IgE antibody 0 |
| | | heavy chain |
| | | variable region |
| | | FR4 |
| SEQ ID NO: 19 | GYSFTSYW | Amino Acid |
| | | Sequence of |
| | | anti-Her2/Neu |
| | | IgE antibody 0 |
| | | heavy chain |
| | | variable region |
| | | CDR1 (IMGT) |
| SEQ ID NO: 20 | IYPGDSDT | Amino Acid |
| | | Sequence of |
| | | anti-Her2/Neu |
| | | IgE antibody 0 |
| | | heavy chain |
| | | variable region |
| | | CDR2 (IMGT) |
| SEQ ID NO: 21 | ARHDVGYCTDRTCAKWPEYFQH | Amino Acid |
| | | Sequence of |
| | | anti-Her2/Neu |
| | | IgE antibody 0 |
| | | heavy chain |
| | | variable region |
| | | CDR3 (IMGT) |
| SEQ ID NO: 22 | KLAATMEWSWVFLFFLSVTTGVHSQVQLVQSGAEVKKPGESL | Amino Acid |
| | KISCKGSGYSFTSYWIAWVRQMPGKGLEYMGLIYPGESDTKY | Sequence of |
| | SPSFQGQVTISVDKSVSTAYLQWSSLKPSDSAVYFCARHDVG | anti-Her2/Neu |
| | YCTDRTCAKWPEYFQHWGQGTLVTVSSASTQSPSVFPLTRCC | IgE antibody 1 |
| | KNIPSNATSVTLGCLATGYFPEPVMVTWDTGSLNGTTMTLPA | Full length |
| | TTLTLSGHYATISLLTVSGAWAKQMFTCRVAHTPSSTDWVDN | heavy chain |
| | KTFSVCSRDFTPPTVKILQSSCDGGGHFPPTIQLLCLVSGYT | |
| | PGTINITWLEDGQVMDVDLSTASTTQEGELASTQSELTLSQK | |
| | HWLSDRTYTCQVTYQGHTFEDSTKKCADSNPRGVSAYLSRPS | |
| | PFDLFIRKSPTITCLVVDLAPSKGTVNLTWSRASGKPVNHST | |
| | RKEEKQRNGTLTVTSTLPVGTRDWIEGETYQCRVTHPHLPRA | |
| | LMRSTTKTSGPRAAPEVYAFATPEWPGSRDKRTLACLIQNFM | |
| | PEDISVQWLHNEVQLPDARHSTTQPRKTKGSGFFVFSRLEVT | |
| | RAEWEQKDEFICRAVHEAASPSQTVQRAVSVNPGK | |
| SEQ ID NO: 23 | VQLVQSGAEVKKPGESLKISCKGSGYSFTSYWIAWVRQMPGK | Amino Acid |
| | GLEYMGLIYPGESDTKYSPSFQGQVTISVDKSVSTAYLQWSS | Sequence of |
| | LKPSDSAVYFCARHDVGYCTDRTCAKWPEYFQHWGQGTLVTV | anti-Her2/Neu |
| | | IgE antibody 1 |
| | | heavy chain |
| | | variable region |
| | | from FR1 to FR4 |
| SEQ ID NO: 24 | VQLVQSGAEVKKPGESLKISCKGS | Amino Acid |
| | | Sequence of |
| | | anti-Her2/Neu |
| | | IgE antibody 1 |
| | | heavy chain |
| | | variable region |
| | | FR1 |
| SEQ ID NO: 25 | IAWVRQMPGKGLEYMGL | Amino Acid |
| | | Sequence of |
| | | anti-Her2/Neu |
| | | IgE antibody 1 |
| | | heavy chain |
| | | variable region |
| | | FR2 |
| SEQ ID NO: 26 | KYSPSFQGQVTISVDKSVSTAYLQWSSLKPSDSAVYFC | Amino Acid |
| | | Sequence of |
| | | anti-Her2/Neu |
| | | IgE antibody 1 |
| | | heavy chain |
| | | variable region |
| | | FR3 |
| SEQ ID NO: 27 | FQHWGQGTLVTV | Amino Acid |
| | | Sequence of |
| | | anti-Her2/Neu |
| | | IgE antibody 1 |
| | | heavy chain |
| | | variable region |
| | | FR4 |
| SEQ ID NO: 28 | GYSFTSYW | Amino Acid |
| | | Sequence of |
| | | anti-Her2/Neu |
| | | IgE antibody 1 |
| | | heavy chain |
| | | variable region |
| | | CDR1 (IMGT) |
| SEQ ID NO: 29 | IYPGESDT | Amino Acid |
| | | Sequence of |
| | | anti-Her2/Neu |
| | | IgE antibody 1 |
| | | heavy chain |
| | | variable region |
| | | CDR2 (IMGT) |
| SEQ ID NO: 30 | ARHDVGYCTDRTCAKWPEY | Amino Acid |
| | | Sequence of |
| | | anti-Her2/Neu |
| | | IgE antibody 1 |
| | | heavy chain |
| | | variable region |
| | | CDR3 (IMGT) |
| SEQ ID NO: 31 | KLAATMEWSWVFLFFLSVTTGVHSQVQLVQSGAEVKKPGESL | Amino Acid |
| | KISCKGSGYSFTSYWIAWVRQMPGKGLEYMGLIYPGQSDTKY | Sequence of |
| | SPSFQGQVTISVDKSVSTAYLQWSSLKPSDSAVYFCARHDVG | anti-Her2/Neu |
| | YCTDRTCAKWPEYFQHWGQGTLVTVSSASTQSPSVFPETRCC | IgE antibody 2 |
| | KNIPSNATSVTLGCLATGYFPEPVMVTWDTGSLNGTTMTLPA | Full length |
| | TTLTLSGHYATISLLTVSGAWAKQMFTCRVAHTPSSTDWVDN | heavy chain |
| | KTFSVCSRDFTPPTVKILQSSCDGGGHFPPTIQLLCLVSGYT | |
| | PGTINITWLEDGQVMDVDLSTASTTQEGELASTQSELTLSQK | |
| | HWLSDRTYTCQVTYQGHTFEDSTKKCADSNPRGVSAYLSRPS | |
| | PFDLFIRKSPTITCLVVDLAPSKGTVNLTWSRASGKPVNHST | |
| | RKEEKQRNGTLTVTSTLPVGTRDWIEGETYQCRVTHPHLPRA | |
| | LMRSTTKTSGPRAAPEVYAFATPEWPGSRDKRTLACLIQNFM | |
| | PEDISVQWLHNEVQLPDARHSTTQPRKTKGSGFFVFSRLEVT | |
| | RAEWEQKDEFICRAVHEAASPSQTVQRAVSVNPGK | |
| SEQ ID NO: 32 | VQLVQSGAEVKKPGESLKISCKGSGYSFTSYWIAWVRQMPGK | Amino Acid |
| | GLEYMGLIYPGQSDTKYSPSFQGQVTISVDKSVSTAYLQWSS | Sequence of |
| | LKPSDSAVYFCARHDVGYCTDRTCAKWPEYFQHWGQGTLVTV | anti-Her2/Neu |
| | | IgE antibody 2 |
| | | heavy chain |
| | | variable region |
| | | from FR1 to FR4 |
| SEQ ID NO: 33 | VQLVQSGAEVKKPGESLKISCKGS | Amino Acid |
| | | Sequence of |
| | | anti-Her2/Neu |
| | | IgE antibody 2 |
| | | heavy chain |
| | | variable region |
| | | FR1 |
| SEQ ID NO: 34 | IAWVRQMPGKGLEYMGL | Amino Acid |
| | | Sequence of |
| | | anti-Her2/Neu |
| | | IgE antibody 2 |
| | | heavy chain |
| | | variable region |
| | | FR2 |
| SEQ ID NO: 35 | KYSPSFQGQVTISVDKSVSTAYLQWSSLKPSDSAVYFC | Amino Acid |
| | | Sequence of |
| | | anti-Her2/Neu |
| | | IgE antibody 2 |
| | | heavy chain |
| | | variable region |
| | | FR3 |
| SEQ ID NO: 36 | WGQGTLVTV | Amino Acid |
| | | Sequence of |
| | | anti-Her2/Neu |
| | | IgE antibody 2 |
| | | heavy chain |
| | | variable region |
| | | FR4 |
| SEQ ID NO: 37 | GYSFTSYW | Amino Acid |
| | | Sequence of |
| | | anti-Her2/Neu |
| | | IgE antibody 2 |
| | | heavy chain |
| | | variable region |
| | | CDR1 (IMGT) |
| SEQ ID NO: 38 | IYPGQSDT | Amino Acid |
| | | Sequence of |
| | | anti-Her2/Neu |
| | | IgE antibody 2 |
| | | heavy chain |
| | | variable region |
| | | CDR2 (IMGT) |
| SEQ ID NO: 39 | ARHDVGYCTDRTCAKWPEYFQH | Amino Acid |
| | | Sequence of |
| | | anti-Her2/Neu |
| | | IgE antibody 2 |
| | | heavy chain |
| | | variable region |
| | | CDR3 (IMGT) |
| SEQ ID NO: 40 | KLAATMEWSWVFLFFLSVTTGVHSQVQLVQSGAEVKKPGESL | Amino Acid |
| | KISCKGSGYSFTSYWIAWVRQMPGKGLEYMGLIYPGSSDTKY | Sequence of |
| | SPSFQGQVTISVDKSVSTAYLQWSSLKPSDSAVYFCARHDVG | anti-Her2/Neu |
| | YCTDRTCAKWPEYFQHWGQGTLVTVSSASTQSPSVFPETRCC | IgE antibody 3 |
| | KNIPSNATSVTLGCLATGYFPEPVMVTWDTGSLNGTTMTLPA | Full length |
| | TTLTLSGHYATISLLTVSGAWAKQMFTCRVAHTPSSTDWVDN | heavy chain |
| | KTFSVCSRDFTPPTVKILQSSCDGGGHFPPTIQLLCLVSGYT | |
| | PGTINITWLEDGQVMDVDLSTASTTQEGELASTQSELTLSQK | |
| | HWLSDRTYTCQVTYQGHTFEDSTKKCADSNPRGVSAYLSRPS | |
| | PFDLFIRKSPTITCLVVDLAPSKGTVNLTWSRASGKPVNHST | |
| | RKEEKQRNGTLTVTSTLPVGTRDWIEGETYQCRVTHPHLPRA | |
| | LMRSTTKTSGPRAAPEVYAFATPEWPGSRDKRTLACLIQNFM | |
| | PEDISVQWLHNEVQLPDARHSTTQPRKTKGSGFFVFSRLEVT | |
| | RAEWEQKDEFICRAVHEAASPSQTVQRAVSVNPGK | |
| SEQ ID NO: 41 | QVQLVQSGAEVKKPGESLKISCKGSGYSFTSYWIAWVRQMPG | Amino Acid |
| | KGLEYMGLIYPGSSDTKYSPSFQGQVTISVDKSVSTAYLQWS | Sequence of |
| | SLKPSDSAVYFCARHDVGYCTDRTCAKWPEYFQHWGQGTLVT | anti-Her2/Neu |
| | V | IgE antibody 3 |
| | | heavy chain |
| | | variable region |
| | | from FR1 to FR4 |
| SEQ ID NO: 42 | QVQLVQSGAEVKKPGESLKISCKGS | Amino Acid |
| | | Sequence of |
| | | anti-Her2/Neu |
| | | IgE antibody 3 |
| | | heavy chain |
| | | variable region |
| | | FR1 |
| SEQ ID NO: 43 | IAWVRQMPGKGLEYMGL | Amino Acid |
| | | Sequence of |
| | | anti-Her2/Neu |
| | | IgE antibody 3 |
| | | heavy chain |
| | | variable region |
| | | FR2 |
| SEQ ID NO: 44 | KYSPSFQGQVTISVDKSVSTAYLQWSSLKPSDSAVYFC | Amino Acid |
| | | Sequence of |
| | | anti-Her2/Neu |
| | | IgE antibody 3 |
| | | heavy chain |
| | | variable region |
| | | FR3 |
| SEQ ID NO: 45 | WGQGTLVTV | Amino Acid |
| | | Sequence of |
| | | anti-Her2/Neu |
| | | IgE antibody 3 |
| | | heavy chain |
| | | variable region |
| | | FR4 |
| SEQ ID NO: 46 | GYSFTSYW | Amino Acid |
| | | Sequence of |
| | | anti-Her2/Neu |
| | | IgE antibody 3 |
| | | heavy chain |
| | | variable region |
| | | CDR1 (IMGT) |
| SEQ ID NO: 47 | IYPGSSDT | Amino Acid |
| | | Sequence of |
| | | anti-Her2/Neu |
| | | IgE antibody 3 |
| | | heavy chain |
| | | variable region |
| | | CDR2 (IMGT) |
| SEQ ID NO: 48 | ARHDVGYCTDRTCAKWPEYFQH | Amino Acid |
| | | Sequence of |
| | | anti-Her2/Neu |
| | | IgE antibody 3 |
| | | heavy chain |
| | | variable region |
| | | CDR3 (IMGT) |
| SEQ ID NO: 49 | KLAATMEWSWVFLFFLSVTTGVHSQVQLVQSGAEVKKPGESL | Amino Acid |
| | KISCKGSGYSFTSYWIAWVRQMPGKGLEYMGLIYPGDTDTKY | Sequence of |
| | SPSFQGQVTISVDKSVSTAYLQWSSLKPSDSAVYFCARHDVG | anti-Her2/Neu |
| | YCTDRTCAKWPEYFQHWGQGTLVTVSSASTQSPSVFPETRCC | IgE antibody 4 |
| | KNIPSNATSVTLGCLATGYFPEPVMVTWDTGSLNGTTMTLPA | Full length |
| | TTLTLSGHYATISLLTVSGAWAKQMFTCRVAHTPSSTDWVDN | heavy chain |
| | KTFSVCSRDFTPPTVKILQSSCDGGGHFPPTIQLLCLVSGYT | |
| | PGTINITWLEDGQVMDVDLSTASTTQEGELASTQSELTLSQK | |
| | HWLSDRTYTCQVTYQGHTFEDSTKKCADSNPRGVSAYLSRPS | |
| | PFDLFIRKSPTITCLVVDLAPSKGTVNLTWSRASGKPVNHST | |
| | RKEEKQRNGTLTVTSTLPVGTRDWIEGETYQCRVTHPHLPRA | |
| | LMRSTTKTSGPRAAPEVYAFATPEWPGSRDKRTLACLIQNFM | |
| | PEDISVQWLHNEVQLPDARHSTTQPRKTKGSGFFVFSRLEVT | |
| | RAEWEQKDEFICRAVHEAASPSQTVQRAVSVNPGK | |
| SEQ ID NO: 50 | VQLVQSGAEVKKPGESLKISCKGSGYSFTSYWIAWVRQMPGK | Amino Acid |
| | GLEYMGLIYPGDTDTKYSPSFQGQVTISVDKSVSTAYLQWSS | Sequence of |
| | LKPSDSAVYFCARHDVGYCTDRTCAKWPEYFQHWGQGTLVTV | anti-Her2/Neu |
| | | IgE antibody 4 |
| | | heavy chain |
| | | variable region |
| | | from FR1 to FR4 |
| SEQ ID NO: 51 | VQLVQSGAEVKKPGESLKISCKGS | Amino Acid |
| | | Sequence of |
| | | anti-Her2/Neu |
| | | IgE antibody 4 |
| | | heavy chain |
| | | variable region |
| | | FR1 |
| SEQ ID NO: 52 | IAWVRQMPGKGLEYMGL | Amino Acid |
| | | Sequence of |
| | | anti-Her2/Neu |
| | | IgE antibody 4 |
| | | heavy chain |
| | | variable region |
| | | FR2 |
| SEQ ID NO: 53 | KYSPSFQGQVTISVDKSVSTAYLQWSSLKPSDSAVYFC | Amino Acid |
| | | Sequence of |
| | | anti-Her2/Neu |
| | | IgE antibody 4 |
| | | heavy chain |
| | | variable region |
| | | FR3 |
| SEQ ID NO: 54 | WGQGTLVTV | Amino Acid |
| | | Sequence of |
| | | anti-Her2/Neu |
| | | IgE antibody 4 |
| | | heavy chain |
| | | variable region |
| | | FR4 |
| SEQ ID NO: 55 | GYSFTSYW | Amino Acid |
| | | Sequence of |
| | | anti-Her2/Neu |
| | | IgE antibody 4 |
| | | heavy chain |
| | | variable region |
| | | CDR1 (IMGT) |
| SEQ ID NO: 56 | IYPGDTDT | Amino Acid |
| | | Sequence of |
| | | anti-Her2/Neu |
| | | IgE antibody 4 |
| | | heavy chain |
| | | variable region |
| | | CDR2 (IMGT) |
| SEQ ID NO: 57 | ARHDVGYCTDRTCAKWPEYFQH | Amino Acid |
| | | Sequence of |
| | | anti-Her2/Neu |
| | | IgE antibody 4 |
| | | heavy chain |
| | | variable region |
| | | CDR3 (IMGT) |
| SEQ ID NO: 58 | KLAATMEWSWVFLFFLSVTTGVHSQVQLVQSGAEVKKPGESL | Amino Acid |
| | KISCKGSGYSFTSYWIAWVRQMPGKGLEYMGLIYPGDIDTKY | Sequence of |
| | SPSFQGQVTISVDKSVSTAYLQWSSLKPSDSAVYFCARHDVG | anti-Her2/Neu |
| | YCTDRTCAKWPEYFQHWGQGTLVTVSSASTQSPSVFPETRCC | IgE antibody 5 |
| | KNIPSNATSVTLGCLATGYFPEPVMVTWDTGSLNGTTMTLPA | Full length |
| | TTLTLSGHYATISLLTVSGAWAKQMFTCRVAHTPSSTDWVDN | heavy chain |
| | KTFSVCSRDFTPPTVKILQSSCDGGGHFPPTIQLLCLVSGYT | |
| | PGTINITWLEDGQVMDVDLSTASTTQEGELASTQSELTLSQK | |
| | HWLSDRTYTCQVTYQGHTFEDSTKKCADSNPRGVSAYLSRPS | |
| | PFDLFIRKSPTITCLVVDLAPSKGTVNLTWSRASGKPVNHST | |
| | RKEEKQRNGTLTVTSTLPVGTRDWIEGETYQCRVTHPHLPRA | |
| | LMRSTTKTSGPRAAPEVYAFATPEWPGSRDKRTLACLIQNFM | |
| | PEDISVQWLHNEVQLPDARHSTTQPRKTKGSGFFVFSRLEVT | |
| | RAEWEQKDEFICRAVHEAASPSQTVQRAVSVNPGK | |
| SEQ ID NO: 59 | VQLVQSGAEVKKPGESLKISCKGSGYSFTSYWIAWVRQMPGK | Amino Acid |
| | GLEYMGLIYPGDIDTKYSPSFQGQVTISVDKSVSTAYLQWSS | Sequence of |
| | LKPSDSAVYFCARHDVGYCTDRTCAKWPEYFQHWGQGTLVTV | anti-Her2/Neu |
| | | IgE antibody 5 |
| | | heavy chain |
| | | variable region |
| | | from FR1 to FR4 |
| SEQ ID NO: 60 | VQLVQSGAEVKKPGESLKISCKGS | Amino Acid |
| | | Sequence of |
| | | anti-Her2/Neu |
| | | IgE antibody 5 |
| | | heavy chain |
| | | variable region |
| | | FR1 |
| SEQ ID NO: 61 | IAWVRQMPGKGLEYMGL | Amino Acid |
| | | Sequence of |
| | | anti-Her2/Neu |
| | | IgE antibody 5 |
| | | heavy chain |
| | | variable region |
| | | FR2 |
| SEQ ID NO: 62 | KYSPSFQGQVTISVDKSVSTAYLQWSSLKPSDSAVYFC | Amino Acid |
| | | Sequence of |
| | | anti-Her2/Neu |
| | | IgE antibody 5 |
| | | heavy chain |
| | | variable region |
| | | FR3 |
| SEQ ID NO: 63 | WGQGTLVTV | Amino Acid |
| | | Sequence of |
| | | anti-Her2/Neu |
| | | IgE antibody 5 |
| | | heavy chain |
| | | variable region |
| | | FR4 |
| SEQ ID NO: 64 | GYSFTSYW | Amino Acid |
| | | Sequence of |
| | | anti-Her2/Neu |
| | | IgE antibody 5 |
| | | heavy chain |
| | | variable region |
| | | CDR1 (IMGT) |
| SEQ ID NO: 65 | IYPGDIDT | Amino Acid |
| | | Sequence of |
| | | anti-Her2/Neu |
| | | IgE antibody 5 |
| | | heavy chain |
| | | variable region |
| | | CDR2 (IMGT) |
| SEQ ID NO: 66 | ARHDVGYCTDRTCAKWPEYFQH | Amino Acid |
| | | Sequence of |
| | | anti-Her2/Neu |
| | | IgE antibody 5 |
| | | heavy chain |
| | | variable region |
| | | CDR3 (IMGT) |
| SEQ ID NO: 67 | AAGCTTGCCGCCACCATGTCTGTGCCTACACAGGTTCTGGGA | Anti-Her2/Neu |
| | CTGCTGCTGCTGTGGCTGACCGATGCTAGATGCCAGTCCGTG | IgE antibody |
| | CTGACCCAGCCTCCTTCTGTTTCTGCTGCCCCTGGCCAGAAA | Full length |
| | GTGACCATCTCTTGCTCCGGCTCCTCCTCCAACATCGGCAAC | light chain |
| | AACTACGTGTCCTGGTATCAGCAGCTGCCCGGCACAGCTCCC | 0 |
| | AAACTGCTGATCTACGACCACACCAACAGACCTGCCGGCGTG | |
| | CCCGATAGATTCTCCGGCTCTAAGTCTGGCACCTCTGCCAGC | |
| | CTGGCCATCTCCGGCTTTAGATCTGAGGACGAGGCCGACTAC | |
| | TACTGCGCCTCTTGGGACTATACCCTGTCCGGCTGGGTTTTC | |
| | GGCGGAGGCACAAAAGTGACCGTCCTAGGTCAACCCAAGGCC | |
| | GCTCCCAGCGTGACCCTGTTCCCCCCCAGCAGCGAGGAGCTG | |
| | CAGGCCAACAAGGCCACCCTGGTGTGTCTGATCAGCGACTTC | |
| | TACCCAGGCGCCGTGACCGTGGCCTGGAAGGCCGACAGCAGC | |
| | CCCGTGAAGGCCGGCGTGGAGACCACCACCCCCAGCAAGCAG | |
| | AGCAACAACAAGTACGCCGCCAGCAGCTACCTGAGCCTGACC | |
| | CCAGAGCAGTGGAAGAGCCACAGGAGCTACAGCTGCCAGGTC | |
| | ACCCACGAGGGCAGCACCGTGGAAAAGACCGTGGCCCCAACC | |
| | GAGTGCTCCTGATGAATTC | |
| SEQ ID NO: 68 | AAGCTTGCCGCCACCATGTCTGTGCCTACACAGGTTCTGGGA | Anti-Her2/Neu |
| | CTGCTGCTGCTGTGGCTGACCGATGCTAGATGCCAGTCCGTG | IgE antibody |
| | CTGACCCAGCCTCCTTCTGTTTCTGCTGCCCCTGGCCAGAAA | Full length |
| | GTGACCATCTCTTGCTCCGGCTCCTCCTCCAACATCGGCAAC | light chain |
| | AACTACGTGTCCTGGTATCAGCAGCTGCCCGGCACAGCTCCC | 1 |
| | AAACTGCTGATCTACGACCACACCAACAGACCTGCCGGCGTG | |
| | CCCGATAGATTCTCCGGCTCTAAGTCTGGCACCTCTGCCAGC | |
| | CTGGCCATCTCCGGCTTTAGATCTGAGGACGAGGCCGACTAC | |
| | TACTGCGCCTCTTGGGACTATACCCTGTCCGGCTGGGTTTTC | |
| | GGCGGAGGCACAAAAGTGACCGTCCTAGGTCAACCCAAGGCC | |
| | GCTCCCAGCGTGACCCTGTTCCCCCCCAGCAGCGAGGAGCTG | |
| | CAGGCCAACAAGGCCACCCTGGTGTGTCTGATCAGCGACTTC | |
| | TACCCAGGCGCCGTGACCGTGGCCTGGAAGGCCGACAGCAGC | |
| | CCCGTGAAGGCCGGCGTGGAGACCACCACCCCCAGCAAGCAG | |
| | AGCAACAACAAGTACGCCGCCAGCAGCTACCTGAGCCTGACC | |
| | CCAGAGCAGTGGAAGAGCCACAGGAGCTACAGCTGCCAGGTC | |
| | ACCCACGAGGGCAGCACCGTGGAAAAGACCGTGGCCCCAACC | |
| | GAGTGCTCCTGATGAATTC | |
| SEQ ID NO: 69 | AAGCTTGCCGCCACCATGTCTGTGCCTACACAGGTTCTGGGA | Anti-Her2/Neu |
| | CTGCTGCTGCTGTGGCTGACCGATGCTAGATGCCAGTCCGTG | IgE antibody |
| | CTGACCCAGCCTCCTTCTGTTTCTGCTGCCCCTGGCCAGAAA | Full length |
| | GTGACCATCTCTTGCTCCGGCTCCTCCTCCAACATCGGCAAC | light chain |
| | AACTACGTGTCCTGGTATCAGCAGCTGCCCGGCACAGCTCCC | 2 |
| | AAACTGCTGATCTACGACCACACCAACAGACCTGCCGGCGTG | |
| | CCCGATAGATTCTCCGGCTCTAAGTCTGGCACCTCTGCCAGC | |
| | CTGGCCATCTCCGGCTTTAGATCTGAGGACGAGGCCGACTAC | |
| | TACTGCGCCTCTTGGGACTATACCCTGTCCGGCTGGGTTTTC | |
| | GGCGGAGGCACAAAAGTGACCGTCCTAGGTCAACCCAAGGCC | |
| | GCTCCCAGCGTGACCCTGTTCCCCCCCAGCAGCGAGGAGCTG | |
| | CAGGCCAACAAGGCCACCCTGGTGTGTCTGATCAGCGACTTC | |
| | TACCCAGGCGCCGTGACCGTGGCCTGGAAGGCCGACAGCAGC | |
| | CCCGTGAAGGCCGGCGTGGAGACCACCACCCCCAGCAAGCAG | |
| | AGCAACAACAAGTACGCCGCCAGCAGCTACCTGAGCCTGACC | |
| | CCAGAGCAGTGGAAGAGCCACAGGAGCTACAGCTGCCAGGTC | |
| | ACCCACGAGGGCAGCACCGTGGAAAAGACCGTGGCCCCAACC | |
| | GAGTGCTCCTGATGAATTC | |
| SEQ ID NO: 70 | AAGCTTGCCGCCACCATGTCTGTGCCTACACAGGTTCTGGGA | Anti-Her2/Neu |
| | CTGCTGCTGCTGTGGCTGACCGATGCTAGATGCCAGTCCGTG | IgE antibody |
| | CTGACCCAGCCTCCTTCTGTTTCTGCTGCCCCTGGCCAGAAA | Full length |
| | GTGACCATCTCTTGCTCCGGCTCCTCCTCCAACATCGGCAAC | light chain |
| | AACTACGTGTCCTGGTATCAGCAGCTGCCCGGCACAGCTCCC | 3 |
| | AAACTGCTGATCTACGACCACACCAACAGACCTGCCGGCGTG | |
| | CCCGATAGATTCTCCGGCTCTAAGTCTGGCACCTCTGCCAGC | |
| | CTGGCCATCTCCGGCTTTAGATCTGAGGACGAGGCCGACTAC | |
| | TACTGCGCCTCTTGGGACTATACCCTGTCCGGCTGGGTTTTC | |
| | GGCGGAGGCACAAAAGTGACCGTCCTAGGTCAACCCAAGGCC | |
| | GCTCCCAGCGTGACCCTGTTCCCCCCCAGCAGCGAGGAGCTG | |
| | CAGGCCAACAAGGCCACCCTGGTGTGTCTGATCAGCGACTTC | |
| | TACCCAGGCGCCGTGACCGTGGCCTGGAAGGCCGACAGCAGC | |
| | CCCGTGAAGGCCGGCGTGGAGACCACCACCCCCAGCAAGCAG | |
| | AGCAACAACAAGTACGCCGCCAGCAGCTACCTGAGCCTGACC | |
| | CCAGAGCAGTGGAAGAGCCACAGGAGCTACAGCTGCCAGGTC | |
| | ACCCACGAGGGCAGCACCGTGGAAAAGACCGTGGCCCCAACC | |
| | GAGTGCTCCTGATGAATTC | |
| SEQ ID NO: 71 | AAGCTTGCCGCCACCATGTCTGTGCCTACACAGGTTCTGGGA | Anti-Her2/Neu |
| | CTGCTGCTGCTGTGGCTGACCGATGCTAGATGCCAGTCCGTG | IgE antibody |
| | CTGACCCAGCCTCCTTCTGTTTCTGCTGCCCCTGGCCAGAAA | Full length |
| | GTGACCATCTCTTGCTCCGGCTCCTCCTCCAACATCGGCAAC | light chain |
| | AACTACGTGTCCTGGTATCAGCAGCTGCCCGGCACAGCTCCC | 4 |
| | AAACTGCTGATCTACGACCACACCAACAGACCTGCCGGCGTG | |
| | CCCGATAGATTCTCCGGCTCTAAGTCTGGCACCTCTGCCAGC | |
| | CTGGCCATCTCCGGCTTTAGATCTGAGGACGAGGCCGACTAC | |
| | TACTGCGCCTCTTGGGACTATACCCTGTCCGGCTGGGTTTTC | |
| | GGCGGAGGCACAAAAGTGACCGTCCTAGGTCAACCCAAGGCC | |
| | GCTCCCAGCGTGACCCTGTTCCCCCCCAGCAGCGAGGAGCTG | |
| | CAGGCCAACAAGGCCACCCTGGTGTGTCTGATCAGCGACTTC | |
| | TACCCAGGCGCCGTGACCGTGGCCTGGAAGGCCGACAGCAGC | |
| | CCCGTGAAGGCCGGCGTGGAGACCACCACCCCCAGCAAGCAG | |
| | AGCAACAACAAGTACGCCGCCAGCAGCTACCTGAGCCTGACC | |
| | CCAGAGCAGTGGAAGAGCCACAGGAGCTACAGCTGCCAGGTC | |
| | ACCCACGAGGGCAGCACCGTGGAAAAGACCGTGGCCCCAACC | |
| | GAGTGCTCCTGATGAATTC | |
| SEQ ID NO: 72 | AAGCTTGCCGCCACCATGTCTGTGCCTACACAGGTTCTGGGA | Anti-Her2/Neu |
| | CTGCTGCTGCTGTGGCTGACCGATGCTAGATGCCAGTCCGTG | IgE antibody |
| | CTGACCCAGCCTCCTTCTGTTTCTGCTGCCCCTGGCCAGAAA | Full length |
| | GTGACCATCTCTTGCTCCGGCTCCTCCTCCAACATCGGCAAC | light chain |
| | AACTACGTGTCCTGGTATCAGCAGCTGCCCGGCACAGCTCCC | 5 |
| | AAACTGCTGATCTACGACCACACCAACAGACCTGCCGGCGTG | |
| | CCCGATAGATTCTCCGGCTCTAAGTCTGGCACCTCTGCCAGC | |
| | CTGGCCATCTCCGGCTTTAGATCTGAGGACGAGGCCGACTAC | |
| | TACTGCGCCTCTTGGGACTATACCCTGTCCGGCTGGGTTTTC | |
| | GGCGGAGGCACAAAAGTGACCGTCCTAGGTCAACCCAAGGCC | |
| | GCTCCCAGCGTGACCCTGTTCCCCCCCAGCAGCGAGGAGCTG | |
| | CAGGCCAACAAGGCCACCCTGGTGTGTCTGATCAGCGACTTC | |
| | TACCCAGGCGCCGTGACCGTGGCCTGGAAGGCCGACAGCAGC | |
| | CCCGTGAAGGCCGGCGTGGAGACCACCACCCCCAGCAAGCAG | |
| | AGCAACAACAAGTACGCCGCCAGCAGCTACCTGAGCCTGACC | |
| | CCAGAGCAGTGGAAGAGCCACAGGAGCTACAGCTGCCAGGTC | |
| | ACCCACGAGGGCAGCACCGTGGAAAAGACCGTGGCCCCAACC | |
| | GAGTGCTCCTGATGAATTC | |
| SEQ ID NO: 73 | KLAATMSVPTQVLGLLLLWLTDARCQSVLTQPPSVSAAPGQK | Amino Acid |
| | VTISCSGSSSNIGNNYVSWYQQLPGTAPKLLIYDHTNRPAGV | Sequence of |
| | PDRFSGSKSGTSASLAISGFRSEDEADYYCASWDYTLSGWVF | anti-Her2/Neu |
| | GGGTKVTVLGQPKAAPSVTLFPPSSEELQANKATLVCLISDF | IgE antibody 0 |
| | YPGAVTVAWKADSSPVKAGVETTTPSKQSNNKYAASSYLSLT | full length |
| | PEQWKSHRSYSCQVTHEGSTVEKTVAPTECS | light chain |
| | | variable region |
| SEQ ID NO: 74 | QSVLTQPPSVSAAPGQKVTISCSGSSSNIGNNYVSWYQQLPG | Amino Acid |
| | TAPKLLIYDHTNRPAGVPDRFSGSKSGTSASLAISGFRSEDE | Sequence of |
| | ADYYCASWDYTLSGWVFGGGTKVTVL | anti-Her2/Neu |
| | | IgE antibody 0 |
| | | light chain |
| | | variable region |
| | | from FR1 to FR4 |
| SEQ ID NO: 75 | QSVLTQPPSVSAAPGQKVTISCSGS | Amino Acid |
| | | Sequence of |
| | | anti-Her2/Neu |
| | | IgE antibody 0 |
| | | light chain |
| | | variable region |
| | | FR1 |
| SEQ ID NO: 76 | VSWYQQLPGTAPKLLIY | Amino Acid |
| | | Sequence of |
| | | anti-Her2/Neu |
| | | IgE antibody 0 |
| | | light chain |
| | | variable region |
| | | FR2 |
| SEQ ID NO: 77 | NRPAGVPDRFSGSKSGTSASLAISGFRSEDEADYYC | Amino Acid |
| | | Sequence of |
| | | anti-Her2/Neu |
| | | IgE antibody 0 |
| | | light chain |
| | | variable region |
| | | FR3 |
| SEQ ID NO: 78 | FGGGTKVTVL | Amino Acid |
| | | Sequence of |
| | | anti-Her2/Neu |
| | | IgE antibody 0 |
| | | light chain |
| | | variable region |
| | | FR4 |
| SEQ ID NO: 79 | SSNIGNNY | Amino Acid |
| | | Sequence of |
| | | anti-Her2/Neu |
| | | IgE antibody 0 |
| | | light chain |
| | | variable region |
| | | CDR1 (IMGT) |
| SEQ ID NO: 80 | DHT | Amino Acid |
| | | Sequence of |
| | | anti-Her2/Neu |
| | | IgE antibody 0 |
| | | light chain |
| | | variable region |
| | | CDR2 (IMGT) |
| SEQ ID NO: 81 | ASWDYTLSGWV | Amino Acid |
| | | Sequence of |
| | | anti-Her2/Neu |
| | | IgE antibody 0 |
| | | light chain |
| | | variable region |
| | | CDR3 (IMGT) |
| SEQ ID NO: 82 | KLAATMSVPTQVLGLLLLWLTDARCQSVLTQPPSVSAAPGQK | Amino Acid |
| | VTISCSGSSSNIGNNYVSWYQQLPGTAPKLLIYDHTNRPAGV | Sequence of |
| | PDRFSGSKSGTSASLAISGFRSEDEADYYCASWDYTLSGWVF | anti-Her2/Neu |
| | GGGTKVTVLGQPKAAPSVTLFPPSSEELQANKATLVCLISDF | IgE antibody 1 |
| | YPGAVTVAWKADSSPVKAGVETTTPSKQSNNKYAASSYLSLT | full length |
| | PEQWKSHRSYSCQVTHEGSTVEKTVAPTECS | light chain |
| | | variable region |
| SEQ ID NO: 83 | QSVLTQPPSVSAAPGQKVTISCSGSSSNIGNNYVSWYQQLPG | Amino Acid |
| | TAPKLLIYDHTNRPAGVPDRFSGSKSGTSASLAISGFRSEDE | Sequence of |
| | ADYYCASWDYTLSGWVFGGGTKVTVL | anti-Her2/Neu |
| | | IgE antibody 1 |
| | | light chain |
| | | variable region |
| | | from FR1 to FR4 |
| SEQ ID NO: 84 | QSVLTQPPSVSAAPGQKVTISCSGS | Amino Acid |
| | | Sequence of |
| | | anti-Her2/Neu |
| | | IgE antibody 1 |
| | | light chain |
| | | variable region |
| | | FR1 |
| SEQ ID NO: 85 | SWYQQLPGTAPKLLIY | Amino Acid |
| | | Sequence of |
| | | anti-Her2/Neu |
| | | IgE antibody 1 |
| | | light chain |
| | | variable region |
| | | FR2 |
| SEQ ID NO: 86 | NRPAGVPDRFSGSKSGTSASLAISGFRSEDEADYYC | Amino Acid |
| | | Sequence of |
| | | anti-Her2/Neu |
| | | IgE antibody 1 |
| | | light chain |
| | | variable region |
| | | FR3 |
| SEQ ID NO: 87 | FGGGTKVTVL | Amino Acid |
| | | Sequence of |
| | | anti-Her2/Neu |
| | | IgE antibody 1 |
| | | light chain |
| | | variable region |
| | | FR4 |
| SEQ ID NO: 88 | SSNIGNNYV | Amino Acid |
| | | Sequence of |
| | | anti-Her2/Neu |
| | | IgE antibody 1 |
| | | light chain |
| | | variable region |
| | | CDR1 (IMGT) |
| SEQ ID NO: 89 | DHT | Amino Acid |
| | | Sequence of |
| | | anti-Her2/Neu |
| | | IgE antibody 1 |
| | | light chain |
| | | variable region |
| | | CDR2 (IMGT) |
| SEQ ID NO: 90 | ASWDYTLSGWV | Amino Acid |
| | | Sequence of |
| | | anti-Her2/Neu |
| | | IgE antibody 1 |
| | | light chain |
| | | variable region |
| | | CDR3 (IMGT) |
| SEQ ID NO: 91 | KLAATMSVPTQVLGLLLLWLTDARCQSVLTQPPSVSAAPGQK | Amino Acid |
| | VTISCSGSSSNIGNNYVSWYQQLPGTAPKLLIYDHTNRPAGV | Sequence of |
| | PDRFSGSKSGTSASLAISGFRSEDEADYYCASWDYTLSGWVF | anti-Her2/Neu |
| | GGGTKVTVLGQPKAAPSVTLFPPSSEELQANKATLVCLISDF | IgE antibody 2 |
| | YPGAVTVAWKADSSPVKAGVETTTPSKQSNNKYAASSYLSLT | full length |
| | PEQWKSHRSYSCQVTHEGSTVEKTVAPTECS | light chain |
| | | variable region |
| SEQ ID NO: 92 | QSVLTQPPSVSAAPGQKVTISCSGSSSNIGNNYVSWYQQLPG | Amino Acid |
| | TAPKLLIYDHTNRPAGVPDRFSGSKSGTSASLAISGFRSEDE | Sequence of |
| | ADYYCASWDYTLSGWVFGGGTKVTVL | anti-Her2/Neu |
| | | IgE antibody 2 |
| | | light chain |
| | | variable region |
| | | from FR1 to FR4 |
| SEQ ID NO: 93 | QSVLTQPPSVSAAPGQKVTISCSGS | Amino Acid |
| | | Sequence of |
| | | anti-Her2/Neu |
| | | IgE antibody 2 |
| | | light chain |
| | | variable region |
| | | FR1 |
| SEQ ID NO: 94 | VSWYQQLPGTAPKLLIY | Amino Acid |
| | | Sequence of |
| | | anti-Her2/Neu |
| | | IgE antibody 2 |
| | | light chain |
| | | variable region |
| | | FR2 |
| SEQ ID NO: 95 | NRPAGVPDRFSGSKSGTSASLAISGFRSEDEADYYC | Amino Acid |
| | | Sequence of |
| | | anti-Her2/Neu |
| | | IgE antibody 2 |
| | | light chain |
| | | variable region |
| | | FR3 |
| SEQ ID NO: 96 | FGGGTKVTVL | Amino Acid |
| | | Sequence of |
| | | anti-Her2/Neu |
| | | IgE antibody 2 |
| | | light chain |
| | | variable region |
| | | FR4 |
| SEQ ID NO: 97 | SSNIGNNY | Amino Acid |
| | | Sequence of |
| | | anti-Her2/Neu |
| | | IgE antibody 2 |
| | | light chain |
| | | variable region |
| | | CDR1 (IMGT) |
| SEQ ID NO: 98 | DHT | Amino Acid |
| | | Sequence of |
| | | anti-Her2/Neu |
| | | IgE antibody 2 |
| | | light chain |
| | | variable region |
| | | CDR2 (IMGT) |
| SEQ ID NO: 99 | ASWDYTLSGWV | Amino Acid |
| | | Sequence of |
| | | anti-Her2/Neu |
| | | IgE antibody 2 |
| | | light chain |
| | | variable region |
| | | CDR3 (IMGT) |
| SEQ ID NO: 100 | KLAATMSVPTQVLGLLLLWLTDARCQSVLTOPPSVSAAPGQK | Amino Acid |
| | VTISCSGSSSNIGNNYVSWYQQLPGTAPKLLIYDHTNRPAGV | Sequence of |
| | PDRFSGSKSGTSASLAISGFRSEDEADYYCASWDYTLSGWVF | anti-Her2/Neu |
| | GGGTKVTVLGQPKAAPSVTLFPPSSEELQANKATLVCLISDF | IgE antibody 3 |
| | YPGAVTVAWKADSSPVKAGVETTTPSKQSNNKYAASSYLSLT | full length |
| | PEQWKSHRSYSCQVTHEGSTVEKTVAPTECS | light chain |
| | | variable region |
| SEQ ID NO: 101 | QSVLTQPPSVSAAPGQKVTISCSGSSSNIGNNYVSWYQQLPG | Amino Acid |
| | TAPKLLIYDHTNRPAGVPDRFSGSKSGTSASLAISGFRSEDE | Sequence of |
| | ADYYCASWDYTLSGWVFGGGTKVTVL | anti-Her2/Neu |
| | | IgE antibody 3 |
| | | light chain |
| | | variable region |
| | | from FR1 to FR4 |
| SEQ ID NO: 102 | QSVLTQPPSVSAAPGQKVTISCSGS | Amino Acid |
| | | Sequence of |
| | | anti-Her2/Neu |
| | | IgE antibody 3 |
| | | light chain |
| | | variable region |
| | | FR1 |
| SEQ ID NO: 103 | VSWYQQLPGTAPKLLIY | Amino Acid |
| | | Sequence of |
| | | anti-Her2/Neu |
| | | IgE antibody 3 |
| | | light chain |
| | | variable region |
| | | FR2 |
| SEQ ID NO: 104 | NRPAGVPDRFSGSKSGTSASLAISGFRSEDEADYYC | Amino Acid |
| | | Sequence of |
| | | anti-Her2/Neu |
| | | IgE antibody 3 |
| | | light chain |
| | | variable region |
| | | FR3 |
| SEQ ID NO: 105 | FGGGTKVTVL | Amino Acid |
| | | Sequence of |
| | | anti-Her2/Neu |
| | | IgE antibody 3 |
| | | light chain |
| | | variable region |
| | | FR4 |
| SEQ ID NO: 106 | SSNIGNNY | Amino Acid |
| | | Sequence of |
| | | anti-Her2/Neu |
| | | IgE antibody 3 |
| | | light chain |
| | | variable region |
| | | CDR1 (IMGT) |
| SEQ ID NO: 107 | DHT | Amino Acid |
| | | Sequence of |
| | | anti-Her2/Neu |
| | | IgE antibody 3 |
| | | light chain |
| | | variable region |
| | | CDR2 (IMGT) |
| SEQ ID NO: 108 | ASWDYTLSGWV | Amino Acid |
| | | Sequence of |
| | | anti-Her2/Neu |
| | | IgE antibody 3 |
| | | light chain |
| | | variable region |
| | | CDR3 (IMGT) |
| SEQ ID NO: 109 | KLAATMSVPTQVLGLLLLWLTDARCQSVLTQPPSVSAAPGQK | Amino Acid |
| | VTISCSGSSSNIGNNYVSWYQQLPGTAPKLLIYDHTNRPAGV | Sequence of |
| | PDRFSGSKSGTSASLAISGFRSEDEADYYCASWDYTLSGWVF | anti-Her2/Neu |
| | GGGTKVTVLGQPKAAPSVTLFPPSSEELQANKATLVCLISDF | IgE antibody 4 |
| | YPGAVTVAWKADSSPVKAGVETTTPSKQSNNKYAASSYLSLT | full length |
| | PEQWKSHRSYSCQVTHEGSTVEKTVAPTECS | light chain |
| | | variable region |
| SEQ ID NO: 110 | QSVLTQPPSVSAAPGQKVTISCSGSSSNIGNNYVSWYQQLPG | Amino Acid |
| | TAPKLLIYDHTNRPAGVPDRFSGSKSGTSASLAISGFRSEDE | Sequence of |
| | ADYYCASWDYTLSGWVFGGGTKVTVL | anti-Her2/Neu |
| | | IgE antibody 4 |
| | | light chain |
| | | variable region |
| | | from FR1 to FR4 |
| SEQ ID NO: 111 | QSVLTQPPSVSAAPGQKVTISCSGS | Amino Acid |
| | | Sequence of |
| | | anti-Her2/Neu |
| | | IgE antibody 4 |
| | | light chain |
| | | variable region |
| | | FR1 |
| SEQ ID NO: 112 | VSWYQQLPGTAPKLLIY | Amino Acid |
| | | Sequence of |
| | | anti-Her2/Neu |
| | | IgE antibody 4 |
| | | light chain |
| | | variable region |
| | | FR2 |
| SEQ ID NO: 113 | NRPAGVPDRFSGSKSGTSASLAISGFRSEDEADYYC | Amino Acid |
| | | Sequence of |
| | | anti-Her2/Neu |
| | | IgE antibody 4 |
| | | light chain |
| | | variable region |
| | | FR3 |
| SEQ ID NO: 114 | FGGGTKVTVL | Amino Acid |
| | | Sequence of |
| | | anti-Her2/Neu |
| | | IgE antibody 4 |
| | | light chain |
| | | variable region |
| | | FR4 |
| SEQ ID NO: 115 | SSNIGNNY | Amino Acid |
| | | Sequence of |
| | | anti-Her2/Neu |
| | | IgE antibody 4 |
| | | light chain |
| | | variable region |
| | | CDR1 (IMGT) |
| SEQ ID NO: 116 | DHT | Amino Acid |
| | | Sequence of |
| | | anti-Her2/Neu |
| | | IgE antibody 4 |
| | | light chain |
| | | variable region |
| | | CDR2 (IMGT) |
| SEQ ID NO: 117 | ASWDYTLSGWV | Amino Acid |
| | | Sequence of |
| | | anti-Her2/Neu |
| | | IgE antibody 4 |
| | | light chain |
| | | variable region |
| | | CDR3 (IMGT) |
| SEQ ID NO: 118 | KLAATMSVPTQVLGLLLLWLTDARCQSVLTQPPSVSAAPGQK | Amino Acid |
| | VTISCSGSSSNIGNNYVSWYQQLPGTAPKLLIYDHTNRPAGV | Sequence of |
| | PDRFSGSKSGTSASLAISGFRSEDEADYYCASWDYTLSGWVF | anti-Her2/Neu |
| | GGGTKVTVLGQPKAAPSVTLFPPSSEELQANKATLVCLISDF | IgE antibody 5 |
| | YPGAVTVAWKADSSPVKAGVETTTPSKQSNNKYAASSYLSLT | full length |
| | PEQWKSHRSYSCQVTHEGSTVEKTVAPTECS | light chain |
| | | variable region |
| SEQ ID NO: 119 | QSVLTQPPSVSAAPGQKVTISCSGSSSNIGNNYVSWYQQLPG | Amino Acid |
| | TAPKLLIYDHTNRPAGVPDRFSGSKSGTSASLAISGFRSEDE | Sequence of |
| | ADYYCASWDYTLSGWVFGGGTKVTVL | anti-Her2/Neu |
| | | IgE antibody 5 |
| | | light chain |
| | | variable region |
| | | from FR1 to FR4 |
| SEQ ID NO: 120 | QSVLTQPPSVSAAPGQKVTISCSGS | Amino Acid |
| | | Sequence of |
| | | anti-Her2/Neu |
| | | IgE antibody 5 |
| | | light chain |
| | | variable region |
| | | FR1 |
| SEQ ID NO: 121 | VSWYQQLPGTAPKLLIY | Amino Acid |
| | | Sequence of |
| | | anti-Her2/Neu |
| | | IgE antibody 5 |
| | | light chain |
| | | variable region |
| | | FR2 |
| SEQ ID NO: 122 | NRPAGVPDRFSGSKSGTSASLAISGFRSEDEADYYC | Amino Acid |
| | | Sequence of |
| | | anti-Her2/Neu |
| | | IgE antibody 5 |
| | | light chain |
| | | variable region |
| | | FR3 |
| SEQ ID NO: 123 | FGGGTKVTVL | Amino Acid |
| | | Sequence of |
| | | anti-Her2/Neu |
| | | IgE antibody 5 |
| | | light chain |
| | | variable region |
| | | FR4 |
| SEQ ID NO: 124 | SSNIGNNY | Amino Acid |
| | | Sequence of |
| | | anti-Her2/Neu |
| | | IgE antibody 5 |
| | | light chain |
| | | variable region |
| | | CDR1 (IMGT) |
| SEQ ID NO: 125 | DHT | Amino Acid |
| | | Sequence of |
| | | anti-Her2/Neu |
| | | IgE antibody 5 |
| | | light chain |
| | | variable region |
| | | CDR2 (IMGT) |
| SEQ ID NO: 126 | ASWDYTLSGWV | Amino Acid |
| | | Sequence of |
| | | anti-Her2/Neu |
| | | IgE antibody 5 |
| | | light chain |
| | | variable region |
| | | CDR3 (IMGT) |

Italic underlined = amino acid sequence of variable region from FR1 to FR4

1. An antibody or an antigen-binding fragment that binds specifically to Her2/Neu comprising three variable heavy domain complementarity determining regions (CDR) (CDR H1, H2 and H3) wherein the CDR H1, H2, and H3, comprise an amino acid sequence comprising:
CDR H1: GYSFTSYW (SEQ ID NO:1),
CDR H2: IYPGX₁X₂DT, where X₁ is D, E, or Q, X₂ is S, I, or T, and wherein when X₁ is D, X₂ is different than S (SEQ ID NO:2), and
CDR H3: ARHDVGYCTDRTCAKWPEY (SEQ ID NO:3), respectively.

2. The antibody or the antigen-binding fragment of claim 1,
comprising three variable light domain CDR (CDR L1, L2 and L3), wherein the CDR L1, L2, and L3 comprise an amino acid sequence comprising:
CDR L1: SSNIGNNY (SEQ ID NO:4),
CDR L2: DHT (SEQ ID NO:5), and
CDR L3: ASWDYTLSGWV (SEQ ID NO:6), respectively.

3. The antibody or the antigen-binding fragment of claim 1, wherein when X₁ is D, X₂ is I, or T.

4. The antibody or the antigen-binding fragment of claim 1, wherein when X₁ is E, or Q, X₂ is S.

5. The antibody or the antigen-binding fragment of claim 1, wherein X₁ is E and X₂ is S.

6. The antibody or the antigen-binding fragment of claim 1, wherein X₁ is Q and X₂ is S.

7-8. (canceled)

9. The antibody or the antigen-binding fragment of claim 1, further comprising four variable heavy domain framework regions (HFR)(HFR 1, 2, 3 and 4), wherein said HFR 1, 2, 3, and 4 comprise an amino acid sequence comprising:
HFR1:
(SEQ ID NO: 15)
VQLVQSGAEVKKPGESLKISCKGS,
HFR2:
(SEQ ID NO: 16)
IAWWRQMPGKGLEYMGL,
HFR3:
(SEQ ID NO: 17)
KYSPSFQGQVTISVDKSVSTAYLQWSSLKPSDSAVYFC,
and
HFR4:
(SEQ ID NO: 18)
WGQGTLVTV.

10. The antibody or the antigen-binding fragment of claim 1, further comprising four variable light domain framework regions (LFR)(LFR 1, 2, 3 and 4), wherein said LFR 1, 2, 3, and 4 comprise an amino acid sequence, comprising:
LFR1:
(SEQ ID NO: 84)
QSVLTQPPSVSAAPGQKVTISCSGS,
LFR2:
(SEQ ID NO: 85)
VSWYQQLPGTAPKLLIY,
LFR 3:
(SEQ ID NO: 86)
NRPAGVPDRFSGSKSGTSASLAISGFRSEDEADYYC,
and
LFR4:
(SEQ ID NO: 87)
FGGGTKVTVL.

11. The antibody or the antigen-binding fragment of claim 1, comprising a variable heavy domain (V_{H}) comprising amino acid sequence, comprising:
(SEQ ID NO: 23)
VQLVQSGAEVKKPGESLKISCKGSGYSFTSYWIAVRQMPGKGLEYMGL
IYPGESDTKYSPSFQGQVTISVDKSVSTAYLQWSSLKPSDSAVYFCAR
HDVGYCTDRTCAKWPEYFQHWGQGTLVTV.

12. The antibody or the antigen-binding fragment of claim 1, comprising a variable light domain (V_{L}) comprising amino acid sequence, comprising:
(SEQ ID NO: 83)
QSVLTQPPSVSAAPGQKVTISCSGSSSNIGNNYVSWYQQLPGTAPKLL
IYDHTNRPAGVPDRFSGSKSGTSASLAISGFRSEDEADYYCASWDYTL
SGWWFGGGTKVTVL.

13. The antibody or the antigen-binding fragment of claim 1, comprising a variable heavy domain (V_{H}) comprising amino acid sequence, comprising:
(SEQ ID NO: 23)
VQLVQSGAEVKKPGESLKISCKGSGYSFTSYWIAVRQMPGKGLEYMGL
IYPGESDTKYSPSFQGQVTISVDKSVSTAYLQWSSLKPSDSAVYFCAR
HDVGYCTDRTCAKWPEYFQHWGQGTLVTV.
and a variable light domain (V_{L}) comprising amino acid sequence, comprising:
(SEQ ID NO: 83)
QSVLTQPPSVSAAPGQKVTISCSGSSSNIGNNYVSWYQQLPGTAPKLL
IYDHTNRPAGVPDRFSGSKSGTSASLAISGFRSEDEADYYCASWDYTL
SGWWFGGGTKVTVL.

14. The antibody or the antigen-binding fragment of claim 1, wherein the antigen-binding fragment is a single-domain antibody (sdAb), a fragment antigen binding (Fab), a single-chain variable fragment (scFv), or a single-chain fragment antigen binding (scFab).

15. The antibody or the antigen-binding fragment of claim 1, wherein the antibody is an IgA, an IgD, an IgE, an IgG, or an IgM.

16. The antibody or the antigen-binding fragment of claim 15, wherein the antibody is an IgE.

17. The antibody or the antigen-binding fragment of claim 1, wherein the antibody or antigen-binding fragment is humanized or partially humanized.

18. A compound comprising the antibody or the antigen-binding fragment of claim 1 and a functional moiety.

19. The compound of claim 18, wherein the antibody or the antigen-binding fragment is linked to the functional moiety via a peptide linker.

20. The compound of claim 19, wherein the antibody or the antigen-binding fragment is functionally linked to the functional moiety via the peptide linker.

21. The compound of claim 20, wherein the peptide linker comprises about 3 to about 40 amino acid residues.

22. The compound of claim 20, wherein
the peptide linker comprises the amino acid sequence (GGGGS)ₙ or (GGGS)ₙ, wherein n≥1.

23. The compound of claim 18, wherein the antibody or the antigen-binding fragment is fused to a second antibody or antigen-binding fragment operable to bind a target epitope.

24. The compound of claim 18, wherein the antibody or the antigen-binding fragment is linked to a peptide, a polypeptide, a protein, an enzyme, a second antibody, an antibody fragment, a second antigen-binding fragment or a combination of any two or more thereof; wherein each of the antibody or antigen-binding fragment thereof and the linked peptide, polypeptide, protein, enzyme, second antibody, antibody fragment, second antigen-binding fragment, or the combination of any two or more thereof is functional.

25. The compound of claim 24, wherein the antibody fragment is a fragment crystallizable (Fc) region.

26. A composition, comprising the antibody or the antigen-binding fragment of claim 1 and a pharmaceutically acceptable diluent, carrier, or excipient.

27-29. (canceled)

30. A method of treating a Her2/Neu associated disease, comprising: administering the antibody or the antigen-binding fragment of claim 1 to a subject in need thereof.

31. The method of claim 30, wherein the Her2/Neu associated disease is a cancer.

32. The method of claim 31, wherein the cancer is ovarian cancer, breast cancer, stomach cancer, lung cancer, uterine cancer, salivary gland cancer, testicular germ cell cancer, bladder cancer, pancreatic cancer, and esophageal cancer.

33-41. (canceled)
